(19) United States
(12) Patent Application Publication
(10) Pub. No.: US 2006/0088897 A1
(43) Pub. Date: Apr. 27, 2006

(54) Modulators

(76) Inventors: Louis Lim, Singapore (SG); Sohail Ahmed, Singapore (SG); Robert Kozma, Singapore (SG)

Correspondence Address: FROMMER LAWRENCE & HAUG, 745 FIFTH AVENUE- 10TH FL., NEW YORK, NY, 10151, US

(21) Appl. No.: 11/232,370

(22) Filed: Sep. 21, 2005

Related U.S. Application Data
(63) Continuation-in-part of application No. PCT/SG04/00063, filed on Mar. 19, 2004.

(30) Foreign Application Priority Data
Mar. 21, 2003 (GB) 0306575.2

(51) Int. Cl. G01N 33/537; C07H 21/04; C12N 9/99

(52) U.S. Cl. 435/7.92; 435/69.2; 435/184; 435/320.1; 435/325; 536/23.2

(57) ABSTRACT

Disclosed is an isoform-specific antagonist of PAK kinase, which is preferably a molecule capable of modulating an interaction between Nck and a PAK isoform. In particular, αPAK, βPAK and γPAK specific inhibitors are disclosed. Also included are methods of treating diseases, preferably characterised by a defect in nerve regeneration, comprising modulating an activity of a PAK kinase isoform, preferably αPAK kinase or γPAK kinase, or both.

### CROSS REFERENCE TO RELATED APPLICATIONS

This application is a continuation-in-part of International Application No. PCT/SG2004/000063, filed Mar. 19, 2004, published as WO 2004/083376 on Sep. 30, 2004, and claiming priority to application GB 0306575.2, filed Mar. 21, 2003.

The foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer 's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the fields of microbiology. It also relates to the fields of medicine, especially therapy and diagnosis.

### BACKGROUND OF THE INVENTION

p21 activated kinases (PAKs), relatives of the yeast Ste20p kinase, were first isolated from brain as proteins that bind to and are activated by Cdc42Hs and Rac1 (Manser et al., 1994). They were subsequently identified in neutrophils (Prigmore et al., 1995; Martin et al., 1995; Knaus et al., 1995). To date, six isoforms have been cloned and sequenced; α, β, γ and δ (PAK1, 3, 2 and 4, respectively; Manser et al., 1995; Teo et al. 1995; Martin et al., 1995; Abo, et. al., 1998) and PAK5 and 6. γPAK is ubiquitous while α and β have a more restricted expression pattern (Manser and Lim, 1999). PAKs are multi-domain proteins that contain, a kinase domain, polyproline stretches (P1-P4), a Cdc42Hs/Rac1 binding site, and a kinase autoinhibitory site (Manser et al., 1994; Zhao et al., 1998).

Two of the polyproline stretches, P1 and P4, interact with the adaptor protein Nck (Bokoch et al., 1996; Galisteo et al., 1996) and the Rac1 exchange factor PIX, respectively, (Manser et al., 1998) via SH3 domains. PAK1-3 are structurally distinct from PAK4-6 in that the latter group lack the Nck binding site and the autoinhibitory site. In mammalian cells overexpression studies suggest that PAK plays a role in cell morphology pathways downstream of Cdc42Hs/Rac1 (Manser et. al., 1997; Zhao et. al., 1998; Sells et. al., 1997), however, this is opposed by studies with p21-effector site mutants (Lamarche et. al., 1996).

The PAK isoforms α, β and γ comprise the same domain structure but have different sequence identities, ranging from 100% in the kinase domain to approx. 95% in the N-terminal. PAK 4, 5 and 6 have 100% identity in the kinase domain but do not the Nck binding site.

To date, no one to our knowledge has disclosed isoform specific inhibition of PAK isoforms.

### SUMMARY OF THE INVENTION

According to a first aspect of the present invention, we provide an isoform specific antagonist of PAK kinase.

Preferably, the isoform specific antagonist of PAK kinase comprises a molecule capable of modulating an interaction between an SH3 containing polypeptide and a PAK isoform. Preferably, the isoform specific antagonist of PAK kinase comprises a molecule capable of preventing or interfering with the binding of Nck, preferably Nckα or Nckβ, to a PAK isoform. Preferably, the isoform specific antagonist of PAK kinase comprises a peptide, preferably comprising a sequence from a PAK kinase isoform, preferably a sequence having an accession number U23443, O88643 or Q13153 (αPAK), U33314, CAD42791 or O75914 (βPAK) or Q64303, AN65624, or NP_{—}002568 (γPAK).

Preferably, the peptide comprises an Nck binding domain. Preferably, the peptide comprises an isoform specific domain. Preferably, the peptide comprises a N-terminal portion of a PAK kinase isoform, preferably residues 1 to 21, 2 to 21, 1 to 20 or 2 to 20 of a PAK kinase isoform. Preferably, the peptide is selected from the group consisting of: EDKPPAPPMRNTSMI (αP1) (SEQ ID NO: 1), SNNGLDIQDKPPAPPMRNTS (αP2) (SEQ ID NO: 2), SDSLDNEEKPPAPPLRMNSN (βP2) (SEQ ID NO: 3) and SDNGELEDKPPAPPVRMSST (γP2) (SEQ ID NO: 4). Preferably, the peptide comprises a sequence from Nck, preferably a PAK binding portion of Nck.

Preferably, the isoform specific antagonist of PAK kinase comprises an antibody.

Preferably, the antibody is selected from the group consisting of: antibody sc-882, antibody sc-1871, antibody sc-1872 and antibody sc-7117 (Santa Cruz Biotechnology, Inc). Preferably, the isoform specific antagonist of PAK kinase is capable of at least one activity selected from the group consisting of: inducing loss of a cell adhesion complex, activating cell spreading, activating cell extension, activating neurite outgrowth, prevention of filopodia formation, and prevention of lamellipodia formation.

Preferably, the isoform specific antagonist of PAK kinase comprises a αPAK isoform specific antagonist. Preferably, it is capable of stimulating neurite outgrowth (neuritogenesis). Preferably, it is capable of stimulating nerve regeneration.

Preferably, the isoform specific antagonist of PAK kinase comprises a γPAK isoform specific antagonist. Preferably, it is capable of inhibiting axonal guidance. Preferably, it is capable of allowing neurite outgrowth across an attractive/repulsive boundary, preferably a laminin/CSPG boundary.

There is provided, according to a second aspect of the present invention, a peptide having the sequence EDKPPAPPMRNTSMI (αP1) (SEQ ID NO: 1), SNNGLDIQDKPPAPPMRNTS (αP2) (SEQ ID NO: 2), SDSLDNEEKPPAPPLRMNSN (βP2) (SEQ ID NO: 3) and SDNGELEDKPPAPPVRMSST (γP2) (SEQ ID NO: 4).

We provide, according to a third aspect of the present invention, a nucleic acid comprising a sequence capable of encoding an isoform specific antagonist of PAK kinase. Preferably, the isoform specific antagonist of PAK kinase comprises an isoform specific antagonist of PAK or a peptide, both as described.

As a fourth aspect of the present invention, there is provided an expression vector comprising a nucleic acid sequence as set out above.

We provide, according to a fifth aspect of the present invention, a method of inhibiting an activity of an isoform of PAK kinase, the method comprising contacting an isoform of PAK kinase with an isoform specific antagonist of PAK kinase.

Preferably, the isoform of PAK kinase is contacted with an isoform specific antagonist of PAK kinase, a peptide, a nucleic acid or an expression vector as set out.

The present invention, in a sixth aspect, provides a method of inhibiting an activity of an αPAK kinase isoform, the method comprising contacting an αPAK kinase with an αPAK isoform specific antagonist; a method of stimulating neurite outgrowth, the method comprising contacting a cell with an αPAK isoform specific antagonist; and a method of stimulating nerve regeneration, the method comprising contacting a cell with an αPAK isoform specific antagonist.

Preferably, the αPAK isoform specific antagonist comprises a peptide EDKPPAPPMRNTSMI (αP1) (SEQ ID NO: 1), SNNGLDIQDKPPAPPMRNTS (αP2) (SEQ ID NO: 2) or an antibody sc-882 (Santa Cruz Biotechnology, Inc).

In a seventh aspect of the present invention, there is provided a method of inhibiting an activity of an γPAK kinase isoform, the method comprising contacting an γPAK kinase with an γPAK isoform specific antagonist.

According to an eighth aspect of the present invention, we provide a method of inhibiting axonal guidance, the method comprising contacting a cell with a γPAK isoform specific antagonist.

We provide, according to a ninth aspect of the invention, a method of enabling neurite outgrowth across an attractive/repulsive boundary, preferably a laminin/CSPG boundary, the method comprising contacting a cell with a γPAK isoform specific antagonist.

Preferably, the γPAK isoform specific antagonist comprises a peptide SDNGELEDKPPAPPVRMSST (γP2) (SEQ ID NO: 4), an antibody sc-1872 or antibody sc-7117 (Santa Cruz Biotechnology, Inc).

The present invention, in a tenth aspect, provides use of an isoform specific antagonist of PAK kinase for specifically inhibiting an activity of an isoform of PAK kinase. Preferably, the isoform specific antagonist of PAK kinase comprises an isoform specific antagonist of PAK kinase, a peptide, a nucleic acid or an expression vector, as described.

As an eleventh aspect of the invention, we provide use of an αPAK isoform specific antagonist of PAK kinase for specifically inhibiting an activity of αPAK kinase.

We further provide use of an αPAK isoform specific antagonist of PAK kinase for stimulating neurite outgrowth; and use of an αPAK isoform specific antagonist of PAK kinase for stimulating nerve regeneration

Preferably, the αPAK isoform specific antagonist comprises a peptide EDKPPAPPMRNTSMI (αP1) (SEQ ID NO: 1), SNNGLDIQDKPPAPPMRNTS (αP2) (SEQ ID NO: 2) or an antibody sc-882 (Santa Cruz Biotechnology, Inc).

We provide, according to a twelfth aspect of the invention, use of an γPAK isoform specific antagonist of PAK kinase for specifically inhibiting an activity of γPAK kinase.

According to a thirteenth aspect of the present invention, we provide use of an γPAK isoform specific antagonist of PAK kinase for inhibiting axonal guidance.

There is provided, according to a fourteenth aspect of the present invention, use of an γPAK isoform specific antagonist of PAK kinase for enabling neurite outgrowth across an attractive/repulsive boundary, preferably a laminin/CSPG boundary.

Preferably, the γPAK isoform specific antagonist comprises a peptide SDNGELEDKPPAPPVRMSST (γP2) (SEQ ID NO: 4), an antibody sc-1872 or antibody sc-7117 (Santa Cruz Biotechnology, Inc).

According to an fifteenth aspect of the present invention, we provide a method of identifying an isoform specific antagonist of PAK kinase, the method comprising: (a) providing a candidate molecule, (b) contacting the candidate molecule with a peptide comprising an Nck binding portion of a PAK isoform, or a fragment, homologue, variant or derivative thereof, and (c) detecting binding of the candidate molecule to the peptide, fragment, homologue, variant or derivative thereof.

We provide, according to a sixteenth aspect of the invention, a method of identifying an isoform specific antagonist of PAK kinase, the method comprising: (a) providing a candidate molecule, (b) contacting a peptide comprising an PAK binding portion of Nck, or a fragment, homologue, variant or derivative thereof, with a candidate molecule, and (c) detecting the binding of the molecule to the PAK binding peptide, fragment, homologue, variant or derivative thereof.

There is provided, in accordance with a seventeenth aspect of the present invention, a method of identifying an isoform specific antagonist of PAK kinase, the method comprising: (a) providing a candidate molecule, (b) contacting the candidate molecule to a peptide comprising an Nck binding portion of a PAK isoform, or a fragment, homologue, variant or derivative thereof, and (c) detecting modulation of activity of the peptide, fragment, homologue, variant or derivative thereof.

Preferably, the PAK isoform is αPAK or γPAK. Preferably, the activity comprises a γPAK isoform specific activity, preferably selected from the group consisting of: maintenance of axonal guidance, and inhibition of neurite outgrowth across an attractive/repulsive boundary, preferably a laminin/CSPG boundary. Preferably, the method further comprises providing Nck, or a fragment, homologue, variant or derivative thereof, and contacting the peptide comprising an Nck binding portion of PAK, or a fragment, homologue, variant or derivative thereof with Nck, or a fragment, homologue, variant or derivative thereof in the presence of the candidate molecule, and selecting candidate molecules which modulate binding between the peptide and Nck. Preferably, the method further comprises isolating or synthesising a selected or identified molecule.

As an eighteenth aspect of the invention, we provide a isoform specific antagonist of PAK kinase identified by such a method.

We provide, according to a nineteenth aspect of the invention, a pharmaceutical composition comprising an isoform specific antagonist of PAK kinase, a peptide, or a nucleic acid as described, together with a pharmaceutically acceptable excipient or carrier.

According to a twentieth aspect of the present invention, we provide use of an isoform specific antagonist of PAK kinase in a method of treatment, prophylaxis or diagnosis of a disease in an individual.

There is provided, according to a twenty-first aspect of the present invention, a method of treatment of an individual suffering or likely to suffer from a disease, the method comprising administering a therapeutically or prophylactically effective amount of an isoform specific antagonist of PAK kinase to the individual.

We provide, according to a twenty-second aspect of the present invention, an isoform specific antagonist of PAK kinase for use in a method of treatment, prophylaxis or diagnosis of a disease in an individual.

According to a twenty-third aspect of the present invention, we provide use of an isoform specific antagonist of PAK kinase in the preparation of a pharmaceutical composition for the treatment of a disease.

Preferably, the disease is characterised by a defect in nerve regeneration or repair.

In preferred embodiments, the disease is selected from the group consisting of: neurodegenerative disorder, an amyloidosis, a tauopathy, Alzheimer's disease, Parkinson's disease (PD), dementia with Lewy Bodies (DLB), frontotemporal dementia (FTD), pallido-ponto-nigral degeneration (PPND), familial progressive subcortical gliosis, and familial multisystem tauopathy (FMT), familial progressive dementia with psychosis, pallido-nigral degeneration and bipolar disorder (manic depressiveness).

Preferably, the isoform specific antagonist of PAK kinase is an αPAK isoform specific antagonist or a γPAK isoform specific antagonist.

According to a twenty-fourth aspect of the present invention, we provide a method of treatment of an individual suffering or likely to suffer from a disease preferably characterised by a defect in nerve regeneration, the method comprising modulating an activity of αPAK kinase and/or γPAK kinase. Preferably, the method comprises inhibiting an interaction, preferably a binding interaction, between αPAK kinase and/or Nck, between γPAK kinase and Nck.

Preferably, the disease is selected from the group consisting of diseases set out above.

We provide, according to a twenty-fifth aspect of the present invention, a method of modulating cellular guidance, preferably axonal guidance, the method comprising exposing a cell, preferably a nerve cell, to an isoform specific antagonist of PAK kinase.

According to a twenty-sixth aspect of the present invention, we provide the use of a γPAK isoform specific antagonist in a method of modulating axonal guidance.

As a twenty-seventh aspect of the invention, we provide use of a γPAK isoform specific antagonist in a method of modulating axonal guidance.

As a twenty-eighth aspect of the invention, we provide use of an isoform specific anti-PAK antibody as an isoform specific antagonist of PAK kinase.

According to a twenty-ninth aspect of the present invention, we provide a combination of a PAK isoform specific antagonist together with a nerve cell. The PAK isoform specific antagonist preferably comprises an αPAK isoform specific antagonist, or a γPAK isoform specific antagonist, or both.

According to a twenty-ninth aspect of the present invention, we provide a kit comprising an isoform specific antagonist of PAK kinase, together with packaging components and instructions for use in nerve regeneration.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### BRIEF DESCRIPTION OF THE DRAWINGS

The patent file contains at least one photograph executed in color. Copies of this patent with color photographs will be provided by the U.S. Patent and Trademark Office upon request and payment of the necessary fee.

Various preferred features and embodiments of the present invention will now be described in more detail by way of non-limiting examples and with reference to the accompanying Figures, in which:

FIG. 1 shows colocalisation of PAK isoforms with vinculin in Bac1 cells. Spreading Bac1 macrophages are doubled stained with PAK isoform (green) antibodies α (A), β (C) and γ (E) and vinculin (red). Colocalisation of PAK with vinculin is shown in blue, in panels (B, D and F) with individual signals removed. Bar=10 μM

FIG. 2 shows the effect of CSF/serum on the cell morphology of Bac1 macrophages. (A) Bac1 cells are grown (24 h) in four different ways and then stained with phalloidin to visualise F-actin. With serum alone (a), without CSF/serum (b), with CSF/serum (c), and with CSF alone (d). (B) Cells are starved of CSF for 24 h and then exposed to gradient of CSF (1.32 nM) through a needle. A time lapse sequence is shown over 30 min. (C) Bac1 macrophages are CSF/serum starved for 24 h and then exposed to a gradient of LPA through a needle. A time lapse sequence is shown over 10 min. (D) Bac1 macrophages starved for serum alone and then exposed to a gradient of CSF through a needle and LPA added to the medium. CSF is added in panel (b) followed by LPA. Panels c-f show process retraction on the side of the cell that is not protected by CSF. Arrows in (B) show the formation of filopodia. Arrows in (C) show process retraction. Bar=10 μM. (E) A model for the antagonistic relationship between Rho family GTPases in Bac1 macrophages is presented. CSF activates both Cdc42Hs and Rac1 (Allen et. al., 1997) to induce the formation of filopodia and lamellipodia at 24 h morphological differentiation is seen. Serum/LPA activates RhoA to induce cell rounding. The addition of CSF antagonises the affect of serum/LPA and vice versa. The balance of the factors CSF vs serum/LPA determines the morphology of the cells.

FIG. 3 shows the effect of C3 and cell differentiation on localisation of αPAK. Bac1 macrophages (A and B) and N1E-115 neuroblastomas (C and D) are stained with αPAK antibodies and vinculin antibodies under different conditions. (A) Growing Bac1 cells with (b, d, f) or without C3 (a, c, e). (B) Starved Bac1 cells. (C and D) Growing and serum starved N1E-115 cells. Colocalisation of αPAK (green) with vinculin (red) is shown in blue (in panel a in C and D). C3 is injected at 100 ng/ml. Bar=5 μM in A and C and 10 μM in B and D.

FIG. 4 shows colocalisation of αPAK with F-actin and vinculin in Swiss 3T3 cells. (A) Cells are stained for αPAK (green) and either phalloidin (a, c, e) or vinculin (b, d, f) both in red. Colocalisation is shown in white (c) or blue (a, b, d, e, f). Cells are freshly plated and allowed to spread for 1 h (a, b), 4 h (c, d) or more than 24 h (e, f) before staining. Insets C′ and D′ show magnification (approx ×16) of areas of the panel. Arrowheads point to areas of colocalisation. (B) A spreading cell left for 4 h is stained for αPAK (green; a-g, white; h) and phalloidin (red). A z-series is shown with panel a being the lowest point and panel f the highest. Panel g shows a magnification of panel b with arrowheads showing colocalisation (blue) of αPAK with stress fibres in red. Bar=10 μM.

FIG. 5 shows colocalisation of PAK α, β and γ isoforms with F-actin in Bac1 macrophages. (A) Cells are starved of both CSF and serum and then doubled stained with PAK isoform (green) antibodies and rhodamine conjugated phalloidin (red). αPAK (A, A′), βPAK (B, B′) and γPAK (C, C′). Panels A′-C′ are magnifications of areas of greatest colocalisation seen in A-C. Colocalisation blue to purple depending on the intensity of green and red signals. (B) Bac1 cells are CSF/serum starved to induce elongation and then doubled stained for (a) F-actin and (b) βPAK colocalisation with F-actin is shown in (c) with individual signals removed. Bar=10 μM.

FIG. 6 shows colocalisation of PAK α, β and γ isoforms with F-actin in N1E-115 cells. Cells are starved for 24 h and then stained for PAK α, β or γ (green) and F-actin (red). Colocalsiation of PAK isoforms and F-actin is shown in panels (b, d, f) in blue with individual signals removed. Arrowheads show areas of colocalisation and allow panel comparison. Bar=10 μM.

FIG. 7 shows the effect of αPAK peptides on cell morphology. Cells are starved for 2-3 h and then either injected with peptides (10 μg/ml) or exposed to peptides with bioporter reagent. Cells are incubated for a further 2 h before being stained with phalloiden and examined by confocal microscopy (as described in the materials and methods section). (A) The upper cells is injected with αP1 (a), or all cells injected with αP1*(b), or exposed to αP2 with bioporter reagent (c). (B) Cells are exposed to peptides in bioporter reagent and cells possessing neurites longer than 1 cell body are scored. Pronounced filopodia; defined as a cell that possesses 5 or more dense staining and robust protrusions that are between 5-10 μM. Pronounced lamellipodia/ruffles; defined as cells that possesses lamellae and/or ruffles that cover 50% or more of the cell. Results are expressed as means+/−SEM of 3 experiments (n=333-426 cells). (C) Cells are exposed to peptides (μg/ml) of αP2 with bioporter reagent and cell possessing neurites longer than 1 cell body length scored. +/−SEM of 3 experiments (n=900). (D) Cells are exposed to bioporter reagent with αP2 and after 6 h double stained with αPAK and vinculin antibodies; (a) Vinculin/αPAK, (b) Vinculin and (c) αPAK. Bar=10 μM. Control bars are with IgG with bioporter/microinjection.

FIG. 8 shows the effect of peptides P2 from αPAK, βPAK, and γPAK on cell morphology. Cells are starved for 2-3 h and then injected with peptides. Cells are then incubated for 2 h before being stained with phalloiden and examined by confocal microscopy (as described in the materials and methods section). (A) Cells are injected with IgG (a), with αP2 (b), with βP2 (c) and γP2 (d). Bar=10 μM. (B) Cells are exposed to bioporter with peptides and presence of neurites, pronounced filopodia and pronounced lamellipodia/ruffles scored (definitions as in FIG. 7). (C) Cells are either exposed to bioporter reagent coated peptides (αP2 and βP2) and then IGF or serum starvation used to activate cells. For γP2 cells are injected with peptide and stimulated with serum. Filopodia and lamellipodia are scored by using time-lapse phase contrast microscopy. +/−SEM of 3 experiments (n=900). Control peptide is taken from the kinase domain and common to the three PAK isoforms.

FIG. 9 shows the effect of peptides on neurite production and axonal guidance. Effect of PAK peptides on neurite morphology. Cells are exposed to control peptide, αP 1*, αP2, βP2, or γP2 and neurite morphology assessed. (B) Cells are plated on coverslips that been treated to create a Laminin/CSGP boundary. After 6 h cells are examined for their ability to project neurites that crossed the substrate boundary. Cells exposed to bioporter coated peptides, (a) αP2, (b) βP2, and (c) γP2. Only cells possessing neurofilament in the neurites and facing a decision at the boundary are considered. Results are means+/−SEM (n=161-263). (C) Cells are exposed to peptides and after 6 h are scored for their ability to cross boundary from Laminin to CSPG.

FIG. 10 shows PAK isoforms co-ordinate cell adherence and cell extension. The relative contribution of PAK isoforms will depend on their degree of activation by factors including; tyrosine kinase activation, Cdc42/Rac, Nck, PI-3 kinase and sphingosine. PAK isoforms link to FA's/FC's via the PIX-GIT-Paxillin complex. PAK isoforms can influence F-actin in a variety of ways; through Lim kinase/ADF, Myosin light chain kinase, and direct phosphorylation of F-actin. αPAK affects the formation and dissembly of FA's/FC's and stress fibres and inhibition of this isoform leads to loss of cell adherence and induction of neurite outgrowth. Other pathways such as via Rho kinase and Citron will also influence levels of cell adherence. γPAK plays a role in fliopodia formaton and inhibition of this isoform leads to defects in guidance. βPak is involved in lamellipodia formation but its inhibition does not affect guidance. The data presented here suggest that PAK isoforms play major and distinct roles in cell motility and can regulate both cell adherence and extension. αPAK may play role on neurite outgrowth while γPAK appears to be important for making guidance decisions.

### DETAILED DESCRIPTION

PAK Isoform Specific Antagonists

The methods and compositions described here are generally concerned with inhibitors of protein activity, preferably enzyme activity. In particular, we describe isoform specific antagonists of protein activity, specifically, antagonists of PAK kinase isoforms. Such antagonists are capable of modulating, including preferably inhibiting, down-regulating or abolishing at least one activity of the protein such as a PAK kinase isoform.

“Isoform specific” means that the antagonist is capable of modulating, preferably down-regulating or abolishing at least one activity of a particular specific isoform of the particular protein or enzyme. Preferably, the antagonist is capable of down-regulating or abolishing that activity, without significant effect on an activity of another isoform. Accordingly, we provide an antagonist which is capable of modulating, preferably down-regulating or abolishing at least one activity of a particular specific isoform of PAK, which we refer to as a “isoform specific antagonist of PAK kinase”.

We demonstrate that it is possible to specifically antagonise or inhibit each of three PAK isoforms, namely, αPAK, βPAK and γPAK, and therefore provide for the first time isoform specific antagonist of PAK kinase. We also demonstrate for the first time that γPAK kinase is essential for axonal guidance.

Accordingly, we provide the use of such isoform specific antagonists of PAK kinase, particularly αPAK and/or γPAK specific antagonists, in the treatment of disease, in particular, diseases characterised by defective neural regeneration or regrowth.

The isoform specific antagonist of PAK kinase may in particular comprise a molecule capable of modulating, preferably preventing or hindering, an interaction between a PAK isoform and a second entity, preferably a second polypeptide. The second polypeptide may preferably comprise a domain capable of interacting with and/or binding to the PAK isoform. The domain may comprise an SH3 binding domain. The second polypeptide may comprise an SH3 containing polypeptide. In particular, the antagonists may prevent or interfere with the binding of the SH3 containing polypeptide to the PAK isoform. The SH3 containing polypeptide in highly preferred embodiments comprises Nck.

The isoform specific antagonists of PAK kinase may in particular comprise peptide isoform specific antagonists of PAK kinase, antibody isoform specific antagonists of PAK kinase, small molecule isoform specific antagonists of PAK kinase, etc.

The isoform specific inhibitors of PAK may be used for various purposes. For example, they may be exposed to a cell (for example a nerve cell) to modulate cellular guidance, for example, axonal guidance. We show that isoform specific inhibitors of PAK, in particular, γPAK, can perform as cell signalling inhibitors of axonal guidance. Preferably, therefore, the isoform specific inhibitors of PAK kinase provided here inhibit sensing of guidance cues, and are preferably used in cases where cellular guidance is mediated by cells (such as nerve cells) sensing a negative signal. In particular, they are preferably not used in cases where the guiding signal defaults of a non-negative signal (i.e., no signal or a positive signal). The isoform specific inhibitors of PAK are therefore useful in treating or preventing diseases or conditions in which a negative cell guidance signal inhibits nerve regeneration or growth.

In other experiments, we show that isoform specific inhibitors of PAK kinase, in particular, αPAK kinase, have neurite outgrowth stimulating properties. Such isoform specific inhibitors of PAK kinase may therefore be used for treating or preventing conditions in which neurite outgrowth is compromised or inhibited. They may in particular be used for nerve regeneration as described in further detail below.

The isoform specific inhibitors of PAK may be used singly or in combination, with each other or with other therapeutics. We therefore disclose combinations of isoform specific inhibitors of PAK, for example, a combination of an αPAK isoform specific inhibitor together with a βPAK isoform specific inhibitor, or a combination of an βPAK isoform specific inhibitor together with a γPAK isoform specific inhibitor, or a combination of an αPAK isoform specific inhibitor together with a γPAK isoform specific inhibitor, or a combination of an αPAK isoform specific inhibitor, a βPAK isoform specific inhibitor, and a γPAK isoform specific inhibitor, each optionally together with other components.

PAK Isoforms

Where reference is made in this document to “PAK”, this should be taken to refer to p21 activated kinase, specifically a p21 Cdc42Hs/Rac 1-activated kinase, for example as described in Manser et al, 1994. The term “PAK activity” should therefore be construed generally, as referring to any activity demonstrated by any PAK kinase.

Reference to a “PAK isoform” should be taken as reference to any isoform of PAK. PAK isoforms, as the term is used here, should preferably include both a kinase domain as well as an Nck binding domain. Kinase domains are well known in the art, and their presence may be easily identified by homology sequence searching.

In highly preferred embodiments, the PAK isoforms that this document is concerned with are those which contain an Nck binding site or Nck binding domain. Such PAK isoforms have been referred to as members of the “PAK I Family”. In such preferred embodiments, the term “PAK isoforms” does not include reference to “PAK II Family” members such as PAK 4, 5, and 6, which don't contain an Nck binding site.

The Nck binding domain may preferably comprise a polyproline domain. More preferably, the PAK isoform comprises a P1 polyproline stretch having for example a sequence PXXPXRXXS (SEQ ID NO: 5), where P=proline, R=arginine, S=serine, and X can be any amino acid, e.g., Alanine, Arginine, Asparagine, Aspartic acid, Cysteine, Glutamine, Glutamic acid, Glycine, Histidine, Isoleucine, Leucine, Lysine, Methionine, Phenylalanine, Proline, Serine, Threonine, Tryptophan, Tyrosine or Valine.

In preferred embodiments, PAK specific isoforms include those containing the high identity in the kinase domain and also comprising a Nck binding domain.

In particular, a PAK isoform should be taken to include αPAK (also known as PAK1, Manser et al., 1995), βPAK (also known as PAK3, Teo et al. 1995), γPAK (also known as PAK2, Martin et al., 1995), ∂PAK (also known as PAK4, Abo, et. al., 1998), PAK5 and PAK6.

The PAK isoforms α, β and γ comprise the same domain structure but have different sequence identities, ranging from 100% in the kinase domain to approx. 95% in the N-terminal. PAK 4, 5 and 6 have 100% identity in the kinase domain but do not the Nck binding site. In highly preferred embodiments, therefore, the PAK isoforms which this document is concerned with are αPAK, βPAK and γPAK, and we therefore disclose in particular, αPAK specific inhibitors, βPAK specific inhibitors and preferably γPAK specific inhibitors.

αPAK/PAK1

αPAK in this document refers to any of the sequences having accession number Q13153 and their homologues (functional and/or by sequence). αPAK preferably also includes fragments, variants and derivatives of any of these sequences.

In particular, an αPAK may comprise the rat Serine/threonine protein kinase known as αPAK (alpha-PAK; PAK-1), and having the accession number U23443. Such an αPAK sequence is presented below:

| | |
|---|---|
| ACCESSION | U23443 |
| VERSION | U23443.1 GI: 2772513 |
| SOURCE | Rattus norvegicus. |
| ORGANISM | Rattus norvegicus |

| | |
|---|---|
| MSNNGLDVQDKPPAPPMRNTSTMIGAGSKDPGTLNHGSKPLPPPEEKKKKDRFYRSILAGDK | (SEQ ID NO: 6) |
| TNKKKEKERPEISLPSDFEHTIHVGFDAVTGEFTGMPEQWARLLQTSNITKSEQKKNPQAVL | |
| DVLEFYNSKKTSNSQKYMSFTDKSAEDYNSSNTLNVKTVSETPAVPPVSEDEDDDDDATPPP | |
| VIAPRPEHTKSVYTRSVIEPLPVTPTRDVATSPISPTENNTTPPDALTRNTEKQKKKPKMSD | |
| EEILEKLRSIVSVGDPKKKYTRFEKIGQGASGTVYTAMDVATGQEVAIKQMNLQQQPKKELI | |
| INEILVMRENKNPNIVNYLDSYLVGDELWVVMEYLAGGSLTDVVTETCMDEGQIAAVCRECL | |
| QALEFLHSNQVIHRDIKSDNILLGMDGSVKLTDFGFCAQITPEQSKRSTMVGTPYWMAPEVV | |
| TRKAYGPKVDIWSLGIMAIEMIEGEPPYLNENPLRALYLIATNGTPELQNPEKLSAIFRDFL | |
| NRCLEMDVEKRGSAKELLQHQFLKIAKPLSSLTPLIAAAKEATKNNH | |

Furthermore, αPAK may comprise the mouse p21-actiavted kinase 1 [Mus musculus] having accession number 088643.

| | |
|---|---|
| ACCESSION | 088643 |
| VERSION | 088643 GI: 6093647 |
| | gi: 3435253, gi: 3435254 |
| SOURCE | Mus musculus (house mouse) |
| ORGANISM | Mus musculus |

| | |
|---|---|
| MSNNGVDIQDKPPAPPMRNTSTMIGAGSKDTGTLNHGSKPLPPNPEEKKKKDRFYRSILPGD | (SEQ ID NO: 7) |
| KTNKKREKERPEISLPSDFEHTIHVGFDAVTGEFTGMPEQWARLLQTSNITKSEQKKNPQAV | |
| LDVLEFYNSKKTSNSKKYMSFTDKSAEDYNSSNTLNVKTVSETPAVPPVSEDDEDDDDDATP | |
| PPVIAPRPEHTKSVYTRSVIEPLPVTPTRDVATSPISPTENNTTPPDALTRNTEKQKKKPKM | |
| SDEEILEKLRSIVSVGDPKKKYTPFEKIGQGASGTVYTAMDVATGQEVAIKQMNLQQQPKKE | |
| LIINEILVMRENKNPNIVNYLDSYLVGDELWVVMEYLAGGSLTDVVTETCMDEGQIAAVCRE | |
| CLQALEFLHSNQVIHRDIKSDNILLGMDGSVKLTDFGFCAQITPEQSK | |

In highly preferred embodiments, αPAK comprises the human Serine/threonine-protein kinase PAK1 having accession number Q13153.

| | |
|---|---|
| ACCESSION | Q13153 |
| VERSION | Q13153 GI: 2499643 |
| SOURCE | Homo sapiens (human) |
| ORGANISM | Homo sapiens |

| | |
|---|---|
| MSNNGLDIQDKPPAPPMRNTSTMIGVGSKDAGTLNHGSKPLPPNPEEKKKKDRFYRSILPGD | (SEQ ID NO: 8) |
| KTNKKKEKERPEISLPSDFEHTIHVGFDAVTGEFTGMPEQWARLLQTSNITKSEQKKNPQAV | |
| LDVLEFYNSKKTSNSQKYMSFTDKSAEDYNSSNALNVKAVSETPAVPPVSEDEDDDDDDATP | |
| PPVIAPRPEHTKSVYTRSVIEPLPVTPTRDVATSPISPTENNTTPPDALTRNTEKQKKKPKM | |
| SDEEILEKLRSIVSVGDPKKKYTRFEKIGQGASGTVYTAMDVATGQEVAIKQMNLQQQPKKE | |
| LIINEILVMRENKNPNIVNYLDSYLVGDELWVVMEYLAGGSLTDVVTETCMDEGQIAAVCRE | |
| CLQALEFLHSNQVIHRDIKSDNILLGMDGSVKLTDFGFCAQITPEQSKRSTMVGTPYWMAPE | |
| VVTRKAYGPKVDIWSLGIMAIEMIEGEPPYLNENPLRALYLIATNGTPELQNPEKLSAIFRD | |
| FLNRCLDMDVEKRGSAKELLQHQFLKIAKPLSSLTPLIAAAKEATKNNH | |

βPAK/PAK3

βPAK in this document refers to a sequence having an accession number 075914and their homologues (functional and/or by sequence) thereof. βPAK preferably also includes fragments, variants and derivatives of any of these sequences.

In particular, a βPAK may comprise the Serine/threonine kinase known as βPAK (beta-PAK or PAK-3), and having the accession number U33314. Such a βPAK sequence is presented below:

| | |
|---|---|
| ACCESSION | U33314 |
| VERSION | U33314.1 GI: 1039424 |
| SOURCE | Rattus norvegicus |
| ORGANISM | Rattus norvegicus |

| | |
|---|---|
| MSDSLDNEEKPPAPPLRMNSNNRDSSALNHSSKPLPMAPEEKNKKARLRSIFPGGGDKTNKK | (SEQ ID NO: 9) |
| KEKERPEISLPSDFEHTIHVGFDAVTGEFTGIPEQWARLLQTSNITKLEQKKNPQAVLDVLK | |
| FYDSKETVNNQKYMSFTSGDKSAHGYIAAHQSNTKTASEPPLAPPVSEEEDEEEEEEEDDNE | |
| PPPVIAPRPEHTKSIYTRSVVESIASPAAPNKEATPPSAENANSSTLYRNTDRQRKKSKMTD | |
| EEILEKLRSIVSVGDPKKKYTRFEKIGQGASGTVYTALDIATGQEVAIKQMNLQQQPKKELI | |
| INEILVMRENKNPNIVNYLDSYLVGDELWVVMEYLAGGSLTDVVTETCMDEGQIAAVCRECL | |
| QALDFLHSNQVIHRDIKSDNILLGMDGSVKLTDFGFCAQITPEQSKRSTMVGTPYWMAPEVV | |
| TRKAYGPKVDIWSLGIMAIEMVEGEPPYLNENPLRALYLIATNGTPELQNPERLSAVFRDFL | |
| NRCLEMDVDRRGSAKELLQHPFLKLAKPLSSLTPLILAAKEAIKNSSR | |

Furthermore, βPAK may comprise the mouse p21-activated kinase 3 [Mus musculus] having accession number CAD42791.

| | |
|---|---|
| ACCESSION | CAD42791 |
| VERSION | CAD42791.1 GI: 21953250 |

| | |
|---|---|
| MSDSLDNEEKPPAPPLRMNSNNRDSSALNHSSKPLPMAPEEKNKKARLRSIFPGGGDKTNKK | (SEQ ID NO: 10) |
| KEKERPEISLPSDFEHTIHVGFDAVTGEFTPDLYGSQMCPGKLPEGIPEQWARLLQTSNITK | |
| LEQKKNPQAVLDVLKFYDSKETVNNQKYMSFTSGDKSAHGYIAARQSNTKTASEPPLAPPVS | |
| EEEDEEEEEEEDDNEPPPVIAPRPEHTKSIYTRSVVESIASPAAPNKEDIPPSAENANSTTL | |
| YRNTDRQRKKSKMTDEEILEKLRSIVSVGDPKKKYTRFEKIGQGASGTVYTALDIATGQEVA | |
| IKQMNLQQQPKKELIINEILVMRENKNPNIVNYLDSYLVGDELWVVMEYLAGGSLTDVVTET | |
| CMDEGQIAAVCRECLQALDFLHSNQVIHRDIKSDNILLGMDGSVKLTDFGFCAQITPEQSKR | |
| STMVGTPYWMAPEVVTRKAYGPKVDIWSLGIMAIEMVEGEPPYLNENPLRALYLIATNGTPE | |
| LQNPERLSAVFRDFLNRCLEMDVDRRGSAKELLQHPFLKLAKPLSSLTPLIIAAKEAIKNSS | |
| R | |

In preferred embodiments, βPAK may comprise the human Serine/threonine-protein kinase PAK3 having accession number 075914.

| | |
|---|---|
| ACCESSION | 075914 |
| VERSION | 075914 GI: 6174887 |
| DBSOURCE | xrefs: gi: 3608385, gi: 3608386 |
| | xrefs (non-sequence databases) HSSPP24941, MIM |
| | 300142, |
| SOURCE | Homo sapiens (human) |
| ORGANISM | Homo sapiens |

| | |
|---|---|
| MSDGLDNEEKPPAPPLRMNSNNRDSSALNHSSKPLPMAPEEKNKKARLRSIFPGGGDKTNKK | (SEQ ID NO: 11) |
| KEKERPEISLPSDFEHTIHVGFDAVTGEFTGIPEQWARLLQTSNITKLEQKKNPQAVLDVLK | |
| FYDSKETVNNQKYMSFTSGDKSAHGYIAAHPSSTKTASEPPLAPPVSEEEDEEEEEEEDENE | |
| PPPVIAPRPEHTKSIYTRSVVESIASPAVPNKEVTPPSAENANSSTLYRNTDRQRKKSKMTD | |
| EEILEKLRSIVSVGDPKKKYTRFEKIGQGASGTVYTALDIATGQEVAIKQMNLQQQPKKELI | |
| INEILVMRENKNPNIVNYLDSYLVGDELWVVMEYLAGGSLTDVVTETCMDEGQIAAVCRECL | |
| QALDFLHSNQVIHRDIKSDNILLGMDGSVKLTDFGFCAQITPEQSKRSTMVGTPYWMAPEVV | |
| TRKAYGPKVDIWSLGIMAIEMVEGEPPYLNENPLRALYLIATNGTPELQNPERLSAVFRDFL | |
| NRCLEMDVDRRGSAKELLQHPFLKLAKPLSSLTPLIIAAKEAIKNSSR | |

γPAK/PAK2

γPAK in this document refers to a sequence having an accession number NP_{—}002568 and their homologues (functional and/or by sequence) thereof. γPAK preferably also includes fragments, variants and derivatives of any of these sequences.

In particular, a γPAK may comprise the Serine/threonine kinase known as Serine/threonine protein kinase γPAK (gamma-PAK or PAK-2), and having the accession number Q64303. Such a γPAK sequence is presented below:

| | |
|---|---|
| ACCESSION | Q64303 |
| VERSION | Q64303 GI: 2499648 |
| SOURCE | Rattus norvegicus |
| ORGANISM | Rattus norvegicus |

| | |
|---|---|
| MSDNGELEDKPPAPPVRMSSTIFSTGGKDPLSANHSLKPLPSVPEEKKPRNKIISIESSTEK | (SEQ ID NO: 12) |
| GSKKKEKERPEISPPSDFEHTIHVGFDAVTGEFTGMPEQWARLLQTSNITKLEQKKNPQAVL | |
| DVLKFYDSNTVKQKYLSFTPPEKDGFPSGTPALNTKGSETSAVVTEEDDDDEDAAPPVIAPR | |
| PDHTKSIYTRSVIDPIPAPVGDSNVDSGAKSSDKQKKKAKMTDEEIMEKLRTIVSIGDPKKK | |
| YTRYEKIGQGASGTVFTATDVALGQEVAIKQINLQKQPKKELIINEILVMKELKNPNIVNFL | |
| DSYLVGDELFVVMEYLAGGSLTDVVTETCMDEAQIAAVCRECLQALEFLHANQVIHRDIKSD | |
| NVLLGMEGSVKLTDFGFCAQITPEQSKRSTMVGTPYWMAPEVVTRKAYGPKVDIWSLGIMAI | |
| EMVEGEPPYLNENPLRALYLIATNGTPELQNPEKLSPIFRDFLNRCLEMDVEKRGSAKELLQ | |
| HPFLKLAKPLSSLTPLILAAKEAMKSNR | |

Furthermore, γPAK may comprise the mouse p21-activated kinase 2 [Mus musculus] having accession number AAN65624.

| | |
|---|---|
| ACCESSION | AAN65624 |
| VERSION | AAN65624.1 GI: 25136580 |
| DBSOURCE | accession AY167030.1 |
| KEYWORDS | . |
| SOURCE | Mus musculus (house mouse) |
| ORGANISM | Mus musculus |

| | |
|---|---|
| MSDNGELEDKPPAPPVRMSSTIFSTGGKDPLSANHSLKPLPSVPEEKKPRNKIISIFSGTEK | (SEQ ID NO: 13) |
| GSKKKEKERPEISPPSDFEHTIHVGFDAVTGEFTGMPEQWARLLQTSNITKLEQKKNPQAVL | |
| DVLKFYDSNTVKQKYLSFTPPEKDGFPSGTPALNTKGSETSAVVTEEDDDDEDAAPPVIAPR | |
| PDHTKSIYTRSVIDPIPAPVGDSNVDSGAKSSDKQKKKAKMTDEEIMEKLRTIVSIGDPKKK | |
| YTRYEKIGQGASGTVFTATDVALGQEVAIKQINLQKQPKKELIINEILVMKELKNPNIVNFL | |
| DSYLVGDELFVVMEYLAGGSLTDVVTETCMDEAQIAAVCRECLQALEFLHANQVIHRDIKSD | |
| NVLLGMEGSVKLTDFGFCAQITPEQSKRSTMVGTPYWMAPEVVTRKAYPKVDIWSLGIMAIE | |
| MVEGEPPYLNENPLRALYLIATNGTPELQNPEKLSPIFRDFLNRCLEMDVEKRGSAELLQHP | |
| FLKLAKPLSSLTPLILAAKEAMKSNR | |

In highly preferred embodiments, γPAK comprises the human Serine/threonine kinase PAK2 having accession number NP_{—}002568.

| | |
|---|---|
| ACCESSION | NP_002568 |
| VERSION | NP_002568.1 GI: 4505599 |
| DBSOURCE | REFSEQ: accession NM_002577.1 |
| SOURCE | Homo sapiens (human) |

| | |
|---|---|
| MSDNGELEDKPPAPPVRMSSTIFSTGGKDPLSANHSLKPLPSVPEEKKPRHKIISIFSGTEK | (SEQ ID NO: 14) |
| GSKKKEKERPEISPPSDFEHTIHVGFDAVTGEFTGMPEQWARLLQTSNITKLEQKKNPQAVL | |
| DVLKFYDSNTVKQKYLSFTPPEKDGLPSGTPALNAKGTEAPAVVTEEEDDDEETAPPVIAPR | |
| PDHTKSIYTRSVIDPVPAPVGDSHVDGAAKSLDKQKKKPKMTDEEIMEKLRTIVSIGDPKKK | |
| YTRYEKIGQGASGTVFTATDVALGQEVAIKQINLQKQPKKELIINEILVMKELKNPNIVNFL | |
| DSYLVGDELFVVMEYLAGGSLTDVVTETACMDEAQIAAVCRECLQALEFLHANQVIHRDIKS | |
| DNVLLGMEGSVKLTDFGFCAQITPEQSKRSTMVGTPYWMAPEVVTRKAYGPKVDIWSLGIMA | |
| IEMVEGEPPYLNENPLRALYLIATNGTPELQNPEKLSPIFRDFLNRCLEMDVEKRGSAKELL | |
| QHPFLKLAKPLSSLTPLIMAAKEAMKSNR | |

PAK Isoform Activities

PAK isoform activities are known in the art, and are also discussed in detail in this document. The isoform specific antagonists of PAK kinase are described in relation to their abilities to inhibit specific isoform activities. Furthermore, assays to identify isoform specific antagonist of PAK kinase (described in detail below) preferably incorporate assays to detect isoform specific activities of PAK isoforms.

The term “PAK isoform activity”, where reference is made to a particular isoform, should be taken to referring to any activity of that particular isoform.

As used in this document, “αPAK activities” or “αPAK specific activities” include localisation with vinculin, localisation in the cell centre, localisation to dynamic RhoA-dependent focal adhesions and stress fibres/F-actin clusters, cell adhesion and cell rounding. αPAK isoform specific antagonists are capable of modulating any or more of these activities.

As used in this document, “βPAK activities” or “βPAK specific activities” include localisation with vinculin, localisation in the peri-nuclear region, localisation in membrane ruffles and lamellipodia, and formation of lamellipodia and membrane ruffles, and βPAK isoform specific antagonists are capable of modulating any one or more of these activities.

As used in this document, “γPAK activities” or “γPAK specific activities” include localisation ni the cell periphery, localisation with rib-like structures and filopodia, filopodia formation and maintenance of axonal guidance, and accordingly, γPAK isoform specific antagonists are capable of modulating any one or more of these activities.

Accordingly, and as an example, a γPAK isoform specific antagonists is one which is capable of down-regulating an activity of γPAK, for example a axonal guidance activity of γPAK, without substantially modulating activity of βPAK or αPAK.

The isoform specific antagonists of PAK kinase described here preferably are capable of modulating an interaction between Nck and a PAK isoform.

By the term “modulation” we mean any change in the interaction between Nck and a PAK isoform; in particular, the antagonists are capable of modulating binding between Nck and a PAK isoform. Preferably, they reduce, abolish, or remove the binding between these two entities. Thus, binding between Nck and the PAK isoform is stronger in the absence of the antagonist, than in its presence. Put another way, the antagonist increases the Km of binding between Nck and PAK isoform.

In preferred embodiments, the PAK isoform binds to Nck in the absence of the antagonist, but not substantially in the presence of the antagonist.

In particular, they are preferably capable of preventing binding of Nck to a PAK isoform, preferably by way of an Nck binding portion of PAK.

The nature of the isoform specific antagonist of PAK kinase does not matter, so long as it exhibits isoform specific antagonism of at least one effect of a PAK isoform. Examples of the nature of such antagonists are provided in greater detail; in particular, isoform specific antagonists of PAK kinase may comprise peptides, immunoglobulins (including antibodies) and small molecules.

Nck

The term “Nck” as it is used in this document should be taken to mean an adaptor protein, as described in any of Bokoch et al., 1996, Galisteo et al., 1996, Chou M M, Fajardo J E, Hanafusa H (1992) The SH2- and SH3-containing Nck protein transforms mammalian fibroblasts in the absence of elevated phosphotyrosine levels. Mol Cell Biol 1992 December;12(12):5834-42.

Preferably, Nck as used here is a SH3 domain containing protein, preferably comprising one SH2 and three SH3 domains. Preferably, Nck as described here comprises the following domains: SH3 domain 1 (residues 2-61), SH3 domain 2 (residues 111-170), SH3 domain 3 (residues 195-257), and SH2 domain (residues 285-377/380). The SH3 domain 2 sequence could for example comprise: AYVKFNYMAE REDELSLIKG TKVIVMEKCS DGWWRGSYNG QVGWFPSNYV TEEGDSPLGD (SEQ ID NO: 15). “SH3 domains” refers to the Src homology motifs found in nonreceptor tyrosine kinases, Ras GTPase-activating protein, phosphatidylinositol 3-kinase, and phospholipase C-gamma.

Two forms of Nck are known in the art, Nckα and Nckβ, having 377 and 380 residues respectively, and the term “Nck” in highly preferred embodiments should be taken to refer to each or either of them, as the context requires.

In preferred embodiments, Nckα refers to the cytoplasmic protein NCK1 (NCK adaptor protein 1) (SH2/SH3 adaptor protein NCK-alpha).

| | |
|---|---|
| ACCESSION | P16333 |
| VERSION | P16333 GI: 127962 |
| DBSOURCE | swissprot: locus NCK1_HUMAN, accession P16333; |
| ORGANISM | Homo sapiens |

| | |
|---|---|
| MAEEVVVVAKFDYVAQQEQELDIKKNERLWLLDDSKSWWRVRNSMNKTGFVPSNYVERKNSA | (SEQ ID NO: 16) |
| RKASIVKNLKDTLGIGKVKRKPSVPDSASPADDSFVDPGERLYDLNMPAYVKFNYMAEREDE | |
| LSLIKGTKVIVMEKCSDGWWRGSYNGQVGWFPSNYVTEEGDSPLGDHVGSLSEKLAAVVNNL | |
| NTGQVLHVVQALYPFSSSNDEELNFEKGDVMDVIEKPENDPEWWKCRKINGMVGLVPKNYVT | |
| VMQNNPLTSGLEPSPPQCDYIRPSLTGKFAGNPWYYGKVTRHQAEMALNERGHEGDFLIRDS | |
| ESSPNDFSVSLKAQGKNKHFKVQLKETVYCIGQRKFSTMEELVEYKKAPIFTSEQGEKLYLV | |
| KHLS | |

In preferred embodiments, Nckβ refers to the cytoplasmic protein NCK2 (NCK adaptor protein 2) (SH2/SH3 adaptor protein NCK-beta) (Nck-2), having the following sequence:

| | |
|---|---|
| ACCESSION | 043639 |
| VERSION | 043639 GI:20532395 |
| ORGANISM | Homo sapiens |
| DBSOURCE | swissprot: locus NCK2_HUMAN, accession 043639; |
| | class: standard. |
| | extra accessions: Q9BWN9, Q9UIC3,created: Oct 16, 2001. |

| | |
|---|---|
| MTEEVIVIAKWDYTAQQDQELDIKKNERLWLLDDSKTWWRVRNAANRTGYVPSNYVERKNSL | (SEQ ID NO: 17) |
| KKGSLVKNLKDTLGLGKTRRKTSARDASPTPSTDAEYPANGSGADRIYDLNIPAFVKFAYVA | |
| EREDELSLVKGSRVTVMEKCSDGWWRGSYNGQIGWFPSNYVLEEVDEAAAESPSFLSLRKGA | |
| SLSNGQGSRVLHVVQTLYPFSSVTEEELNFEKGETMEVIEKPENDPEWWKCKNARGQVGLVP | |
| KNYVVVLSDGPALHPAHAPQISYTGPSSSGRFAGREWYYGNVTRHQAECALNERGVEGDFLI | |
| RDSESSPSDFSVSLKASGKNKHFKVQLVDNVYCIGQRRFHTMDELVEHYKKAPIFTSEHGEK | |
| LYLVRALQ | |

Treatment of Diseases

According to the methods and compositions described here, modulation of activity of αPAK or γPAK kinase, or both, may be used to treat or prevent disease. Particular diseases which are treatable or preventable include neural degenerative diseases as well as diseases associated with defective neural regeneration or regrowth.

Such diseases include Parkinson's disease, Alzheimer's disease, and prion related diseases. Furthermore, diseases include diseases associated with or caused by nerve injury due to ischaemia or anoxia.

We therefore provide for antagonists of PAK isoforms, in particular, αPAK and/or γPAK isoforms, and their use in treatment or prevention (or both) of such diseases.

As shown in the Examples, inhibition of αPAK can be used to stimulate neurite outgrowth (neuritogenesis). Accordingly, αPAK inhibitors may be used for enabling nerve regeneration. In particular, αPAK specific antagonists such as αP2 peptide may be used to treat or prevent nervous injury, for example in central nervous system (CNS) repair and specifically in treatment or prevention of spinal cord injury.

γPAK is shown in the Examples as being essential for axonal guidance. The Examples show that when γPAK activity is reduced or inhibited, for example by exposure to a γPAK specific antagonist, neurites lose their normal inhibitory guidance cues, and are able to grow across a laminin/CSPG boundary. Accordingly, this effect may be used to enable nerve regeneration and regrowth, and down-regulation of γPAK activity (by for example γPAK specific antagonists) may be used to treat diseases.

Furthermore, we disclose the use of isoform specific antagonists of PAK kinase, in particular γPAK kinase, in the treatment of such diseases, in particular, diseases associated with defective neural regeneration or regrowth. The nature of isoform specific antagonists of PAK kinase, and methods to obtain these, have been described in detail above.

For example, modulation of γPAK and/or αPAK activity as described above, and in general isoform specific antagonists of PAK kinase, may be used to treat neural injury, for example spinal cord injury. They may also be used to treat neurodegenerative disorders or diseases, such as an amyloidosis, a tauopathy, Alzheimer's disease, Parkinson's disease (PD), dementia with Lewy Bodies (DLB), frontotemporal dementia (FTD), pallido-ponto-nigral degeneration (PPND), familial progressive subcortical gliosis, and familial multisystem tauopathy (FMT), familial progressive dementia with psychosis, pallido-nigral degeneration and bipolar disorder (manic depressiveness).

Parkinson's Disease and Dementia

Parkinson's disease (PD) and at least one form of dementia (Dementia with Lewy Bodies, or DLB) are caused by the aggregation and incorporation of α-synuclein into intracytoplasmic inclusions called Lewy bodies (Arima et al., 1998; Baba et al., 1998; Mezey et al., 1998; Polymeropoulos, 1998; Spillantini et al., 1998; Trojanowski et al., 1998; Trojanowski and Lee, 1998).

Prion-Related Encephalopathies

Prion-related encephalopathies such as bovine spongiform encephalopathy (BSE, or ‘mad cow disease’) and its human forms Creutzfeldt-Jakob disease (CJD) and kuru are caused by the self-catalysed misfolding and aggregation of metastable proteins known as prions (Forloni, 1996; Forloni et al., 1996; Horwich and Weissman, 1997; Price et al., 1993; Prusiner and Deamond, 1995); several dominantly inherited neurodegenerative diseases including Huntington's disease (HD), X-linked spinal and bulbar muscular atrophy (SBMA), dentatorubral-pallidoluysian atrophy (DRPLA), and at least five genetically distinct forms of spinocerebellar ataxia (SCA types 1, 2, 3, 6 and 7; SCA3 is better known as Machado-Joseph disease, or MJD) are caused by the aggregation and incorporation of proteins or protein fragments containing abnormally expanded glutamine repeats into intranuclear inclusions (Perutz, 1999; Ross, 1997).

Spinal Cord Injury

Isoform specific inhibitors of PAK, in particular of αPAK and γPAK isoforms, are useful in treatment of nerve injuries, in particular spinal cord injury.

The spinal cord plays a fundamental role in allowing the brain to sense and respond to the environment through modulation of skin, muscles and joints as well as other tissues. The spinal cord is enclosed and protected by the vertebrae. If the vertebrae are dislocated the spinal cord can be damaged or severed. Insults to the spinal cord can lead to the devastating disease states such as paraplegia or quadriplegia.

At present, a number of therapeutic strategies are currently being pursued to aid in recovery after spinal cord injury including; the use of 3-D scaffolds to bridge the damaged area and the transplantation of neural stem cells to help regenerate nerves and their connections.

Peptide Antagonists of PAK Isoforms

In one embodiment, the isoform specific antagonist of PAK kinase comprises a peptide, or an entity comprising a peptide (for example, a peptide associated with another entity, which may itself be a peptide). The term “peptide”, as used in this document, refers generally to a polymer of amino acids. It includes, for example, di-peptides, tri-peptides, oligopeptides and polypeptides.

Preferably, the peptide comprises “natural” amino acids, that is to say, amino acids as they exist in peptides in nature. However, modified versions of amino acids, which may be derivatised to change one or more activities or properties, for example, water solubility, are also included.

The peptide antagonist may be made by biochemical methods, for example, protein digestion of native a PAK isoform, or preferably by recombinant DNA methods as known in the art. Accordingly, it will be understood that peptide isoform specific antagonists of PAK kinase specifically include recombinant peptides. The peptide antagonists disclosed also include homologous sequences obtained from any source, for example related viral/bacterial proteins, cellular homologues and synthetic peptides, as well as variants or derivatives thereof. Thus peptide antagonists also include sequences from homologues of PAK isoforms from other species including other microorganisms. Furthermore, homologues from higher animals such as mammals (e.g. mice, rats or rabbits), especially primates, more especially humans are also included.

The peptide isoform specific antagonists of PAK kinase described here in one embodiment are based on PAK kinase, and preferably comprise at least a portion of a PAK kinase isoform. Preferably, a peptide isoform specific antagonist of αPAK comprises at least a portion of an αPAK/PAK1 isoform having the accession number U23443, O88643 or Q13153. Preferably, a peptide isoform specific antagonist of βPAK kinase comprises at least a portion of an βPAK/PAK3 isoform having the accession number U33314, CAD42791 or O75914. Preferably, a peptide isoform specific antagonist of γPAK kinase comprises at least a portion of a γPAK/PAK2 isoform having the accession number Q64303, AN65624, or NP_{—}002568.

Human sequences are preferred. Therefore, a peptide isoform specific antagonist of αPAK preferably comprises at least a portion of an αPAK/PAK1 isoform having the accession number Q13153, a peptide isoform specific antagonist of βPAK kinase comprises at least a portion of an βPAK/PAK3 isoform having the accession number O75914, and a peptide isoform specific antagonist of γPAK kinase comprises at least a portion of a γPAK/PAK2 isoform having the accession number or NP_{—}002568.

Peptide isoform specific antagonists of PAK kinase may also be derived from Nck sequences, so that they comprise at least a portion of Nck. The peptide isoform specific antagonists of PAK kinase of PAK kinase may be based on the sequence of PAK and/or Nck from any organism, but preferably from a higher organism, preferably a chordate, more preferably a vertebrate, even more preferably a mammal, even more preferably a primate, most preferably from a human. For example, Nck and/or PAK kinase sequences from rat, mouse or human may be used.

The peptide isoform specific antagonists of PAK kinase may, in addition to Nck or PAK kinase sequences, comprise other sequences, for example sequences from other proteins or polypeptides. In particular, they may comprise sequences for expression, for tagging, for solubility, and for membrane penetration. Where the peptide isoform specific antagonists of PAK kinase comprise more than one peptide or more than one sequence, the peptides or sequences (for example, the PAK sequences and the other sequences) need not be contiguous, and in particular, need not be joined by peptide bonds. For example, they may be conjugated to each other, by use of crosslinkers as known in the art. However, they are preferably provided as fusion proteins, for example, a fusion protein between GST and a portion of a PAK kinase isoform.

Sequence fragments of PAK kinase or any of its isoforms may be obtained by cleavage, or recombinant expression, or as synthetic peptides. They may be tested for PAK kinase antagonist activity using any suitable assay, as described above and in the Examples. For example, they may be tested for binding to PAK or to Nck, or inhibition of binding between PAK and Nck. In particular, they may be tested for inhibition of axonal guidance using a laminin/CSPG assay. Such sequence fragments which exhibit desired activity may be chosen for use as peptide isoform specific antagonists of PAK kinase.

The peptide isoform specific antagonists of PAK kinase may be of any suitable length, for example, 50, 45, 40, 35, 30, 25, 24, 23, 22, 21, 20, 19, 18, 17, 16, 15, 14, 13, 12, 11, 10 or 9 or fewer residues. Preferably, they comprise or consist of between 21 to 10 amino acid residues, more preferably 21, 20 or 13 amino acid residues.

In particular, the peptide isoform specific antagonists of PAK kinase described here preferably comprise a sequence from an N terminal region of a PAK kinase isoform. For example, they may comprise the first 80, 70, 60, 50, 40, 30, 25, 24, 23, 22, 21, 20, 19, 18, 17, 16, 15, 14, 13, 12, 11 or 10 amino acids of the PAK isoform, for example αPAK, βPAK or γPAK. In preferred embodiments, they comprise the N-terminal 23, 22, 21, 20, 19, 18, 17, 16, 15, 14, 13, 12, 11 or 10 amino acids of the PAK isoform. In further preferred embodiments, they comprise residues 1-13 of the PAK isoform, for example residues 1-13 of αPAK, βPAK or γPAK. In highly preferred embodiments, they comprise residues 1-21, 2-21, 1-20 or 2-20 of the PAK isoform, for example residues 1-21 of αPAK, βPAK or γPAK.

It should be noted that the peptide isoform specific antagonists of PAK kinase need not comprise the extreme N terminal residues or sequences of the PAK kinase isoform, and that they may include “windows” of sub-sequences from the PAK kinase isoform, in particular, sub-sequences of 23, 22, 21, 20, 19, 18, 17, 16, 15, 14, 13, 12, 11 or 10 amino acids starting from the N-terminus. Therefore, they may for example comprise sequences of the lengths as set out above, comprising sequences of for example, residues 1-10, 1-11, 1-12, 1-13, 1-14, 1-15, 1-16, 1-17, 1-18, 1-19, 1-20, 1-21, 1-22, 1-23, 2-10, 2-11, 2-12, 2-13, 2-14, 2-15, 2-16, 2-17, 2-18, 2-19, 2-20, 2-21, 2-22, 2-23, 2-24, 3-11, 3-12, 3-13, 3-14, 3-15, 3-16, 3-17, 3-18, 3-19, 3-20, 3-21, 3-22, 3-23, 3-24, 3-25, 4-12, 4-13, 4-14, 4-15, 4-16, 4-17, 4-18, 4-19, 4-20, 4-21, 4-22, 4-23, 4-24, 4-25, 4-26, 5-13, 5-14, 5-15, 5-16, 5-17, 5-18, 5-19, 5-20, 5-21, 5-22, 5-23, 5-24, 5-25, 5-26, 5-27, 6-14, 6-15, 6-16, 6-17, 6-18, 6-19, 6-20, 6-21, 6-22, 6-23, 6-24, 6-25, 6-26, 6-27, 6-28, 7-15, 7-16, 7-17, 7-18, 7-19, 7-20, 7-21, 7-22, 7-23, 7-24, 7-25, 7-26, 7-27, 7-28, 7-29, 8-16, 8-17, 8-18, 8-19, 8-20, 8-21, 8-22, 8-23, 8-24, 8-25, 8-26, 8-27, 8-28, 8-29, 8-30, 9-17, 9-18, 9-19, 9-20, 9-21, 9-22, 9-23, 9-24, 9-25, 9-26, 9-27, 9-28, 9-29, 9-30, 9-31, 10-18, 10-19, 10-20, 10-21, 10-22, 10-23, 10-24, 10-25, 10-26, 10-27, 10-28, 10-29, 10-30, 10-31, 10-32, etc.

Preferably, the peptide isoform specific antagonists of PAK kinase comprise any portion of 5 or more amino acids, preferably 6, 7, 8, 9 or 10 or more amino acids from the sequences SNNGLDVQDKPPAPPMRNTS (SEQ ID NO: 18), SDNGELEDKPPAPPVRMSST (SEQ ID NO: 4), SDSLDNEEKPPAPPLRMNSNN (SEQ ID NO: 19), SNNGLDIQDKPPAPPMRNTS (SEQ ID NO: 2), SDNGELEDKPPAPPVRMSSTI (SEQ ID NO: 20), SDSLDNEEKPPAPPLRMNSNN (SEQ ID NO: 21), SNNGVDIQDKPPAPPMRNTS (SEQ ID NO: 22), SDNGELEDKPPAPPVRMSSTI (SEQ ID NO: 23) and SDGLDNEEKPPAPPLRMNSNN (SEQ ID NO: 24).

The peptide antagonists may therefore in highly preferred embodiments comprise the sequences EDKPPAPPMRNTSMI (αP1) (SEQ ID NO: 1), SNNGLDIQDKPPAPPMRNTS (αP2) (SEQ ID NO: 2), SDSLDNEEKPPAPPLRMNSN (βP2) (SEQ ID NO: 3) and SDNGELEDKPPAPPVRMSST (γP2) (SEQ ID NO: 4).

In preferred embodiments, an αPAK isoform specific antagonist comprises SNNGLDIQDKPPAPPMRNTS (αP2) (SEQ ID NO: 2). In preferred embodiments, a βPAK isoform specific antagonist comprises SDSLDNEEKPPAPPLRMNSN (βP2) (SEQ ID NO:

3). In preferred embodiments, a γPAK isoform specific antagonist comprises SDNGELEDKPPAPPVRMSST (γP2) (SEQ ID NO: 4).

Peptide antagonists may be designed from PAK isoform sequences or homologues from any species. For example, αPAK, βPAK and γPAK isoform specific peptide antagonists may include the following: (SEQ ID NOS: 25-33, top to bottom, left to right)

| | αPAK peptide | βPAK peptide | γPAK peptide |
|---|---|---|---|
| Organism | antagonist | antagonist | antagonist |
| Rat | SNNGLDVQDKPPAPPMR | SDSLDNEEKPPAPPLRMN | SDNGELEDKPPAPPVRM |
| | NTS | SNN | SSTI |
| Mouse | SNNGVDIQDKPPAPPMR | SDGLDNEEKPPAPPLRMN | SDNGELEDKPPAPPVRM |
| | NTS | SNN | SSTI |
| Human | SNNGLDIQDKPPAPPMR | SDSLDNEEKPPAPPLRMN | SDNGELEDKPPAPPVRM |
| | NTS | SNN | SSTI |

In highly preferred embodiments, the PAK isoform specific peptide antagonists comprise human sequences.

Domains

In preferred embodiments, the peptide isoform specific antagonists of PAK kinase isoforms comprise a portion that is capable of binding to Nck, or a portion that is capable of isoform specificity, or preferably both. In particular, the peptide isoform specific antagonists of PAK kinase may comprise a Nck binding Portion or an Isoform Specific Portion (also referred to as “Nck Binding Domain” and “Isoform Specific Domain” respectively). These domains are described in further detail below. In highly preferred embodiments, the peptide isoform specific antagonists comprise both an Nck binding Portion and an Isoform Specific Portion.

In highly preferred embodiments, the Nck binding domain and the isoform specific domain are derived from the same PAK kinase isoform. However, embodiments in which the Nck binding domain is from one PAK kinase isoform, and the isoform specific domain is from another different PAK kinase isoform, are also included. As an example, we include a peptide isoform specific antagonists of PAK kinase comprising an Nck binding domain from αPAK and an isoform specific domain from γPAK, a peptide isoform specific antagonists of PAK kinase comprising an Nck binding domain from γPAK and an isoform specific domain from αPAK, etc.

The two domains are preferably contiguous—in other words be on the same polypeptide, and may be in either order. For example, arrangements N terminus—Nck binding Portion—Isoform Specific Portion—C terminus and C terminus—Nck binding Portion—Isoform Specific Portion—N terminus are both possible and are included.

Furthermore, non-contiguous forms of peptides are also possible. Any association of a Nck binding domain with a Isoform Specific Domain may be used. For example, the two domains may be coupled, fused, mixed, combined, or otherwise joined to each other. The coupling, etc between the Nck binding domain with a Isoform Specific Domain may be permanent or transient, and may involve covalent or non-covalent interactions (including ionic interactions, hydrophobic forces, Van der Waals interactions, etc). The exact mode of coupling is not important, so long as the peptide is capable of isoform specific antagonism or inhibition of PAK isoform activity.

Coupling technologies are well known in the art. Direct linkage may be achieved by means of a functional group on the agent such as a hydroxyl, carboxy or amino group. Indirect linkage can occur through a linking moiety such as, but not limited to, one or more of bi-functional cross-linking agents, as known in the art.

Nck Binding Domain (“Core Motif”)

In preferred embodiments, peptide isoform specific antagonists of PAK kinase comprise an Nck binding region or Nck binding domain of the PAK isoform, or a portion thereof. The Nck binding domain is believed to mediate binding between PAK or the PAK isoform, to Nck (preferably to a PAK binding portion of Nck), though the methods and compositions described here should not be taken to be bound by this theory.

The Nck binding domain preferably comprises at least 9 residues, but may comprise more.

For example, the Nck binding domain may comprise a polyproline domain. More preferably, the peptide isoform specific antagonists may comprise a P1 polyproline stretch having for example a sequence PXXPXRXXS (SEQ ID NO: 5), where P=proline, R=arginine, S=serine, and X can be any amino acid, e.g., Alanine, Arginine, Asparagine, Aspartic acid, Cysteine, Glutamine, Glutamic acid, Glycine, Histidine, Isoleucine, Leucine, Lysine, Methionine, Phenylalanine, Proline, Serine, Threonine, Tryptophan, Tyrosine or Valine. Needless to say, residues on each or either side of this motif, for example, 1, 2, 3, 4, 5 residues etc, may be included.

Examples of Nck binding domains comprising the motif set out above, and which are preferably included in the peptides, include PAPPMRNTS (SEQ ID NO: 34), PAPPVRMSS (SEQ ID NO: 35), PAPPLRMNS (SEQ ID NO: 36), PAPPMRNTS (SEQ ID NO: 37), PAPPVRMSS (SEQ ID NO: 38), PAPPLRMNS (SEQ ID NO: 39), PAPPMRNTS (SEQ ID NO: 40), PAPPVRMSS (SEQ ID NO: 41) and PAPPLRMNS (SEQ ID NO: 42).

In highly preferred embodiments, the Nck binding domain or Nck binding site comprises an Nckα binding domain, or an Nckβ binding domain.

Isoform Specific Domain

The peptide isoform specific antagonists of PAK kinase may preferably comprise an “Isoform Specific Domain”. The isoform specific domain is believed to mediate the isoform specificity of the peptide antagonist, although again the methods and compositions described here should not be bound by this particular theory.

Homologous or similar regions between PAK isoforms, for example between αPAK, βPAK and γPAK, as well as regions which are not homologous or similar may be identified. Such non-homologous or heterologous regions may be used as a basis for, and preferably as, isoform specific domains for incorporation into the peptide isoform specific antagonists of PAK kinase described here.

Preferably, therefore, a peptide isoform specific antagonist of PAK kinase comprises a portion of PAK kinase which is heterologous, non-homologous or heterogeneous between PAK kinase isoforms. Preferably, the heterogeneous, etc sequence is one which is sufficient, preferably necessary, to confer isoform specificity to the peptide (for convenience, such a sequence is referred to as an “isoform specific domain”). Such heterogeneous regions may be identified by sequence comparison using sequence alignment software, or by eye.

In preferred embodiments, the isoform specific domain comprises a sequence of a PAK isoform which is upstream of the Nck binding site, such as a sequence upstream of a PXXPXRXXS (SEQ ID NO: 5) motif. The isoform specific domain may comprise any suitable number of upstream residues, such as 2, 3, 4, 5, 6, 7, 8, 9, 10, 11, 12, etc. Suitably, all the residues upstream of the Nck binding site, to the N-terminal sequence (or to residue 2) are included. Preferably, the isoform specific domain comprises residues 1-6, 1-7, 1-8, 1-9, 1-10, 1-11, 1-12, 2-6, 2-7, 2-8, 2-9, 2-10, 2-11, 2-12, 2-13, 3-6, 3-7, 3-8, 3-9, 3-10, 3-11, 3-12, 3-13, 3-14, 4-6, 4-7, 4-8, 4-9, 4-10, 4-11, 4-12, 4-13, 4-14, 4-15, 5-6, 5-7, 5-8, 5-9, 5-10, 5-11, 5-12, 5-13, 5-14, 5-15, 5-16, 6-7, 6-8, 6-9, 6-10, 6-11, 6-12, 6-13, 6-14, 6-15, 6-16, 6-17, etc, of any or each of αPAK, αPAK and γPAK.

The number of residues in the isoform specific domain can vary, but in general will be necessary, preferably sufficient, to confer isoform specificity. Where the sequence of the isoform specific domain overlaps with the sequence of the Nck binding domain, the overlapping sequences may be excluded (i.e., the Nck binding domain is directly contiguous with the isoform specific domain), or the extra overlapping sequences may be included.

For example, any of the following isoform specific domains may be included in the peptides to provide the specificity described:

An αPAK isoform specific domain sequence may comprise MSNNGLDVQDKP (SEQ ID NO: 43), MSNNGLDIQDKP (SEQ ID NO: 44), MSNNGVDIQDKP (SEQ ID NO: 45), SNNGLDVQDKP (SEQ ID NO: 46), SNNGLDIQDKP (SEQ ID NO: 47) or SNNGVDIQDKP (SEQ ID NO: 55).

A βPAK isoform specific domain sequence may comprise MSDSLDNEEKP (SEQ ID NO: 48), MSDSLDNEEKPNN (SEQ ID NO: 49), MSDGLDNEEKPNN (SEQ ID NO: 50), SDSLDNEEKP (SEQ ID NO: 51), SDSLDNEEKPNN (SEQ ID NO: 52) or SDGLDNEEKPNN (SEQ ID NO: 53).

A γPAK isoform specific domain sequence may comprise MSDNGELEDKPTI (SEQ ID NO: 54), MSDNGELEDKP (SEQ ID NO: 56), SDNGELEDKPTI (SEQ ID NO: 57) or SDNGELEDKP (SEQ ID NO: 58).

It will be appreciated that combinations of such isoform specific domain sequences may be included. For example, an inhibitor may include two or more such domains for the required double, triple, or multiple specificity.

The peptide isoform specific antagonists of PAK kinase may be used singly or in combination, with each other or with other therapeutics. We therefore disclose a combination of a peptide isoform specific antagonist of αPAK kinase together with a peptide isoform specific antagonist of βPAK kinase, a peptide isoform specific antagonist of αPAK kinase together with a peptide isoform specific antagonist of γPAK kinase, a peptide isoform specific antagonist of βPAK kinase together with a peptide isoform specific antagonist of γPAK kinase, optionally together with other components such as therapeutics. Furthermore, we disclose a combination of a peptide isoform specific antagonist of αPAK kinase, a peptide isoform specific antagonist of βPAK kinase, and a peptide isoform specific antagonist of γPAK kinase, optionally with other components such as therapeutics.

In another embodiment, peptide isoform specific antagonists of PAK kinase may also be derived from Nck sequences, so that they comprise at least a portion of Nck.

For example, peptide isoform specific antagonists of PAK kinase based on Nck sequences may comprise any one or more of Nck SH3 domains, as set out above, or any portion thereof. The peptide isoform specific antagonists of PAK kinase may comprise any one or more of SH3 domain 1 (residues 2-61), SH3 domain 2 (residues 111-170), SH3 domain 3 (residues 195-257). In particular, they may comprise SH3 domain 2 of Nck. The peptide isoform specific antagonists of PAK kinase may therefore comprise an SH3 domain 2 sequence: AYVKFNYMAEREDELSLIKGTKVIVMEKCSDGWWRGSYNGQVGWFPSNYVTEEGDSPLGD (SEQ ID NO: 59), or a portion thereof, preferably a portion capable of binding a PAK kinase isoform.

Antibody Antagonists of PAK Isoforms

Isoform specific antagonists of PAK kinase may also comprise immunoglobulins, including antibodies.

In preferred embodiments, the antibody isoform specific antagonists of PAK kinase are capable of specific binding to an N terminal region of a PAK isoform, for example an N terminal region of αPAK, βPAK or γPAK.

Preferably, the antibody isoform specific antagonist of PAK kinase is capable of specific binding to a PAK isoform. By this, we mean that it is capable of binding to one isoform, but not substantially to other PAK isoforms. The binding site of the antibody may be located in a “heterogeneous” region of PAK, that is to say, a region that is not similar or substantially different between PAK isoforms. For example, this can be an area that is different in sequence, or not homologous or similar, between αPAK, βPAK and γPAK. “Heterogeneous” regions may be identified as discussed above for peptides. A preferred “heterogeneous” region comprises an N terminal region of the PAK isoforms. Therefore, the antibody antagonist may be capable of specific binding to the first 80, 70, 60, 50, 40, 30, 25, 24, 23, 22, 21, 20, 19, 18, 17, 16, 15, 14, 13, 12, 11 or 10 amino acids of the PAK isoform, for example αPAK, βPAK or γPAK. In preferred embodiments, the antibody isoform specific antagonist of PAK kinase may be capable of specific binding to residues 1-13, or more preferably residues 1-21 of the PAK isoform.

The antibody isoform specific antagonists of PAK kinase may be capable of binding to an Nck binding region of the PAK isoform, or a portion thereof. Examples of such regions are set out above. For example, they may be capable of binding to a polyproline rich sequence as set out above, or a portion thereof.

Antibody isoform specific antagonists of PAK kinase may comprise anti-peptide antibodies, and these in particular may be made by immunisation, or phage display selection, or any other means, with EDKPPAPPMRNTSMI (αP1) (SEQ ID NO: 1), SNNGLDIQDKPPAPPMRNTS (αP2) (SEQ ID NO: 2), SDSLDNEEKPPAPPLRMNSN (βP2) (SEQ ID NO: 3) or SDNGELEDKPPAPPVRMSST (γP2) (SEQ ID NO: 4), or any combination of these. In general, the antibodies may be made using standard techniques using any of the peptides disclosed above as peptide isoform specific antagonists of PAK kinase as antigens.

In preferred embodiments, the antibody is selected from the group consisting of the following antibodies made by and available from Santa Cruz Biotechnology, Inc (Santa Cruz Biotechnology, Inc., 2161 Delaware Avenue, Santa Cruz, Calif. 95060, U.S.A.). Any of these may be used as antibody isoform specific inhibitors of PAK kinase.

| Product | Cat. # | Isotype | Epitope |
|---|---|---|---|
| αPAK (N-20) | sc-882 | rabbit IgG | N-terminus |
| βPAK (N-19) | sc-1871 | goat IgG | N-terminus |
| γPAK (N-19) | sc-1872 | goat IgG | N-terminus |
| γPAK (V-19) | sc-7117 | goat IgG | N-terminus |

“Cat#” refers to the catalogue number of the antibody, in the Santa Cruz catalogue.

Small Molecule Antagonists of PAK Isoforms

In addition to peptides and antibodies, small molecules may be used to specifically inhibit PAK isoforms. We therefore disclose small molecule αPAK, βPAK and γPAK inhibitors, as well as assays for screening for these. Small molecule isoform specific antagonists of PAK kinase are screened by detecting modulation, preferably down regulation, of binding or other activity.

By “down-regulation” we include any negative effect on the behaviour being studied; this may be total, or partial. Thus, where binding is being detected, candidate antagonists are capable of reducing, ameliorating, or abolishing the binding between two entities. Preferably, the down-regulation of binding (or any other activity) achieved by the candidate molecule is at least 10%, preferably at least 20%, preferably at least 30%, preferably at least 40%, preferably at least 50%, preferably at least 60%, preferably at least 70%, preferably at least 80%, preferably at least 90%, or more compared to binding (or which ever activity) in the absence of the candidate molecule. Thus, a candidate molecule suitable for use as an antagonist is one which is capable of reducing by 10% more the binding or other activity.

The assays may comprise binding assays. In general, such binding assays involve exposing a PAK isoform polypeptide, nucleic acid, or a fragment, homologue, variant or derivative thereof to a candidate molecule and detecting an interaction or binding between the PAK isoform polypeptide, nucleic acid, or a fragment, homologue, variant or derivative thereof and the candidate molecule. The binding assay may be conducted in vitro, or in vivo. They may be conducted in a test tube, comprising substantially only the components mentioned, or they may be conducted using cell free extracts or more or less purified components. The assays may be conducted within a cell, tissue, organ or organism.

In some embodiments, the assays are conducted on whole organisms rather than cells. Preferably, the organism is one which suffers from a disease as disclosed in this document, or exhibits one or more symptoms of such a disease. The nature etc of candidate molecules are discussed in further detail below, and in particular, they may be in the form of a library, also discussed below.

The candidate molecules are contacted with the test protein or proteins, and an effect detected. In the simplest assay, for example, the candidate molecule is contacted with a PAK isoform, and binding to the PAK isoform tested. An assay may also involve contacting the candidate molecule with a peptide comprising a relevant portion of a PAK isoform, preferably a Nck binding portion of a PAK isoform. Binding of the candidate molecule of the candidate molecule to the peptide is detected. It will be appreciated that fragments, homologues, variants or derivatives of the PAK isoform may be employed, provided that comprise Nck binding activity.

Another binding assay useful in identifying isoform specific antagonists of PAK kinase involves detecting modulation of binding between a peptide comprising a Nck binding portion of PAK, and Nck protein, in the presence of a candidate molecule. Candidate molecules which modulate binding between the peptide and Nck, preferably down-regulate this binding, are chosen.

One type of assay for identifying substances that bind to a polypeptide involves contacting a polypeptide, which is immobilised on a solid support, with a non-immobilised candidate substance determining whether and/or to what extent the polypeptide and candidate substance bind to each other. Alternatively, the candidate substance may be immobilised and the polypeptide non-immobilised. This may be used to detect substances capable of binding to PAK isoforms, or fragments, homologues, variants or derivatives thereof.

In a preferred assay method, the polypeptide is immobilised on beads such as agarose beads. Typically this is achieved by expressing the PAK isoform, or a fragment, homologue, variant or derivative thereof as a GST-fusion protein in bacteria, yeast or higher eukaryotic cell lines and purifying the GST-fusion protein from crude cell extracts using glutathione-agarose beads (Smith and Johnson, 1988). As a control, binding of the candidate substance, which is not a GST-fusion protein, to the immobilised polypeptide is determined in the absence of the polypeptide. The binding of the candidate substance to the immobilised polypeptide is then determined. This type of assay is known in the art as a GST pull down assay. Again, the candidate substance may be immobilised and the polypeptide non-immobilised.

It is also possible to perform this type of assay using different affinity purification systems for immobilising one of the components, for example Ni-NTA agarose and histidine-tagged components.

Binding of the PAK isoform, or a fragment, homologue, variant or derivative thereof to the candidate substance may be determined by a variety of methods well-known in the art. For example, the non-immobilised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Other assays measure the effect of the candidate molecule in the interaction of the PAK isoform with other molecules, such as other proteins. Thus, for example, we provide an assay in which a candidate molecule is contacted with a peptide comprising a Nck binding portion of PAK (or a fragment, etc as described above), and in which modulation of activity of the peptide, fragment, etc is detected. The activity which is modulated may be any PAK activity, or preferably, a PAK isoform specific activity, for example an αPAK, βPAK or γPAK isoform specific activity. Such isoform specific activities are described above in further detail.

In particular, the isoform specific activities which are modulated, preferably down-regulated, include maintenance of axonal guidance, and inhibition of neurite outgrowth across an attractive/repulsive boundary, preferably a laminin/CSPG boundary.

Candidate molecules may be isolated or synthesised by means known in the art, and if needed used for further study.

Candidate Molecules

Suitable candidate molecules for use in the above assays include peptides, especially of from about 5 to 30 or 10 to 25 amino acids in size. Peptides from panels of peptides comprising random sequences or sequences which have been varied consistently to provide a maximally diverse panel of peptides may be used.

Suitable candidate molecules also include antibody products (for example, monoclonal and polyclonal antibodies, single chain antibodies, chimeric antibodies and CDR-grafted antibodies). Furthermore, combinatorial libraries, peptide and peptide mimetics, defined chemical entities, oligonucleotides, and natural product libraries may be screened for activity. The candidate molecules may be used in an initial screen in batches of, for example types of molecules per reaction, and the molecules of those batches which show enhancement or reduction of the activity being assayed tested individually.

Libraries

Libraries of candidate molecules, such as libraries of polypeptides or nucleic acids, may be employed in the methods and compositions described here. Such libraries may be exposed to the PAK peptide, with or without Nck, as described above, and the relevant assay carried out. Depending on the assay, they may also be exposed a cell or organism in the presence of the PAK peptide, with or without Nck.

Selection protocols for isolating desired members of large libraries are known in the art, as typified by phage display techniques. Such systems, in which diverse peptide sequences are displayed on the surface of filamentous bacteriophage (Scott and Smith (1990 supra), have proven useful for creating libraries of antibody fragments (and the nucleotide sequences that encoding them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) supra; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci. U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference). Such techniques may be modified if necessary for the expression generally of polypeptide libraries.

One particularly advantageous approach has been the use of scFv phage-libraries (Bird, R. E., et al. (1988) Science 242: 423-6, Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) supra; Marks et al. (1991) supra; Chiswell et al. (1992) Trends Biotech., 10: 80; Marks et al. (1992) supra). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra), which are incorporated herein by reference.

Alternative library selection technologies include bacteriophage lambda expression systems, which may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use. These expression systems may be used to screen a large number of different members of a library, in the order of about 10⁶ or even more. Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271.

Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection. These and all the foregoing documents also are incorporated herein by reference.

Combinatorial Libraries

Libraries, in particular, libraries of candidate molecules, may suitably be in the form of combinatorial libraries (also known as combinatorial chemical libraries).

A “combinatorial library”, as the term is used in this document, is a collection of multiple species of chemical compounds that consist of randomly selected subunits. Combinatorial libraries may be screened for molecules which are capable of acting as small molecule isoform specific antagonists of PAK kinase.

Various combinatorial libraries of chemical compounds are currently available, including libraries active against proteolytic and non-proteolytic enzymes, libraries of whole-cell oncology and anti-infective targets, among others. A comprehensive review of combinatorial libraries, in particular their construction and uses is provided in Dolle and Nelson (1999), Journal of Combinatorial Chemistry, Vol 1 No 4, 235-282. Reference is also made to Combinatorial peptide library protocols (edited by Shmuel Cabilly, Totowa, N.J. Humana Press, c1998. Methods in Molecular Biology; v. 87).

Further references describing chemical combinatorial libraries, their production and use include those available from the URL http://www.netsci.org/Science/Combichem/, including The Chemical Generation of Molecular Diversity. Michael R. Pavia, Sphinx Pharmaceuticals, A Division of Eli Lilly (Published July, 1995); Combinatorial Chemistry: A Strategy for the Future—MDL Information Systems discusses the role its Project Library plays in managing diversity libraries (Published July, 1995); Solid Support Combinatorial Chemistry in Lead Discovery and SAR Optimization, Adnan M. M. Mjalli and Barry E. Toyonaga, Ontogen Corporation (Published July, 1995); Non-Peptidic Bradykinin Receptor Antagonists From a Structurally Directed Non-Peptide Library. Sarvajit Chakravarty, Babu J. Mavunkel, Robin Andy, Donald J. Kyle*, Scios Nova Inc. (Published July, 1995); Combinatorial Chemistry Library Design using Pharmacophore Diversity Keith Davies and Clive Briant, Chemical Design Ltd. (Published July, 1995); A Database System for Combinatorial Synthesis Experiments—Craig James and David Weininger, Daylight Chemical Information Systems, Inc. (Published July, 1995); An Information Management Architecture for Combinatorial Chemistry, Keith Davies and Catherine White, Chemical Design Ltd. (Published July, 1995); Novel Software Tools for Addressing Chemical Diversity, R. S. Pearlman, Laboratory for Molecular Graphics and Theoretical Modeling, College of Pharmacy, University of Texas (Published June/July, 1996); Opportunities for Computational Chemists Afforded by the New Strategies in Drug Discovery: An Opinion, Yvonne Connolly Martin, Computer Assisted Molecular Design Project, Abbott Laboratories (Published June/July, 1996); Combinatorial Chemistry and Molecular Diversity Course at the University of Louisville: A Description, Arno F. Spatola, Department of Chemistry, University of Louisville (Published June/July, 1996); Chemically Generated Screening Libraries: Present and Future. Michael R. Pavia, Sphinx Pharmaceuticals, A Division of Eli Lilly (Published June/July, 1996); Chemical Strategies For Introducing Carbohydrate Molecular Diversity Into The Drug Discovery Process. Michael J. Sofia, Transcell Technologies Inc. (Published June/July, 1996); Data Management for Combinatorial Chemistry. Maryjo Zaborowski, Chiron Corporation and Sheila H. DeWitt, Parke-Davis Pharmaceutical Research, Division of Warner-Lambert Company (Published November, 1995); and The Impact of High Throughput Organic Synthesis on R&D in Bio-Based Industries, John P. Devlin (Published March, 1996).

Techniques in combinatorial chemistry are gaining wide acceptance among modern methods for the generation of new pharmaceutical leads (Gallop, M. A. et al., 1994, J. Med. Chem. 37:1233-1251; Gordon, E. M. et al., 1994, J. Med. Chem. 37:1385-1401.). One combinatorial approach in use is based on a strategy involving the synthesis of libraries containing a different structure on each particle of the solid phase support, interaction of the library with a soluble receptor, identification of the ‘bead’ which interacts with the macromolecular target, and determination of the structure carried by the identified ‘bead’ (Lam, K. S. et al., 1991, Nature 354:82-84). An alternative to this approach is the sequential release of defined aliquots of the compounds from the solid support, with subsequent determination of activity in solution, identification of the particle from which the active compound was released, and elucidation of its structure by direct sequencing (Salmon, S. E. et al., 1993, Proc. Natl. Acad. Sci. USA 90:11708-11712), or by reading its code (Kerr, J. M. et al., 1993, J. Am. Chem. Soc. 115:2529-2531; Nikolaiev, V. et al., 1993, Pept. Res. 6:161-170; Ohlmeyer, M. H. J. et al., 1993, Proc. Natl. Acad. Sci. USA 90:10922-10926).

Soluble random combinatorial libraries may be synthesized using a simple principle for the generation of equimolar mixtures of peptides which was first described by Furka (Furka, A. et al., 1988, Xth International Symposium on Medicinal Chemistry, Budapest 1988; Furka, A. et al., 1988, 14th International Congress of Biochemistry, Prague 1988; Furka, A. et al., 1991, Int. J. Peptide Protein Res. 37:487-493). The construction of soluble libraries for iterative screening has also been described (Houghten, R. A. et al. 1991, Nature 354:84-86). K. S. Lam disclosed the novel and unexpectedly powerful technique of using insoluble random combinatorial libraries. Lam synthesized random combinatorial libraries on solid phase supports, so that each support had a test compound of uniform molecular structure, and screened the libraries without prior removal of the test compounds from the support by solid phase binding protocols (Lam, K. S. et al., 1991, Nature 354:82-84).

Thus, a library of candidate molecules may be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture).

A library of molecules may include, for example, amino acids, oligopeptides, polypeptides, proteins, or fragments of peptides or proteins; nucleic acids (e.g., antisense; DNA; RNA; or peptide nucleic acids, PNA); aptamers; or carbohydrates or polysaccharides. Each member of the library can be singular or can be a part of a mixture (e.g., a compressed library). The library may contain purified compounds or can be “dirty” (i.e., containing a significant quantity of impurities). Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) may also be used with the methods described here.

In addition to libraries as described above, special libraries called diversity files can be used to assess the specificity, reliability, or reproducibility of the new methods. Diversity files contain a large number of compounds (e.g., 1000 or more small molecules) representative of many classes of compounds that could potentially result in nonspecific detection in an assay. Diversity files are commercially available or can also be assembled from individual compounds commercially available from the vendors listed above.

Candidate Substances

Suitable candidate substances include peptides, especially of from about 5 to 30 or 10 to 25 amino acids in size, based on the sequence of the polypeptides described in the Examples, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for a polypeptide. Furthermore, combinatorial libraries, peptide and peptide mimetics, defined chemical entities, oligonucleotides, and natural product libraries may be screened for activity as inhibitors of binding of a polypeptide to the cell division cycle machinery, for example mitotic/meiotic apparatus (such as microtubules). The candidate substances may be used in an initial screen in batches of, for example 10 substances per reaction, and the substances of those batches which show inhibition tested individually. Candidate substances which show activity in in vitro screens such as those described below can then be tested in whole cell systems, such as mammalian cells which will be exposed to the inhibitor and tested for inhibition of any of the stages of the cell cycle.

Antibodies

The antibody isoform specific antagonist of PAK kinase described above may comprise a monoclonal antibody or a polyclonal antibody. This section discusses processes for the production of monoclonal or polyclonal antibodies which may be used as isoform specific antagonists of PAK kinase.

If polyclonal antibodies are desired, a selected mammal (e.g., mouse, rabbit, goat, horse, etc.) is immunised with an immunogenic polypeptide bearing an epitope(s) from a polypeptide, for example, a peptide derived from a PAK isoform as described above. In particular, immunisation with a heterogeneous region of PAK, for example, an N terminal region, or a Nck binding region of a relevant PAK isoform, for example αPAK, βPAK or γPAK, may be used. Immunisation with PAK isoform whole proteins may also be used.

Serum from the immunised animal is collected and treated according to known procedures. If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order that such antibodies may be made, we also provide polypeptides or fragments thereof haptenised to another polypeptide for use as immunogens in animals or humans.

Monoclonal antibodies directed against epitopes in the polypeptides can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against epitopes from polypeptides are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this document, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in method of detecting polypeptides present in biological samples by a method which comprises: (a) providing an antibody; (b) incubating a biological sample with said antibody under conditions which allow for the formation of an antibody-antigen complex; and (c) determining whether antibody-antigen complex comprising said antibody is formed. By this process, PAK isoforms may be detected in tissues or samples.

Suitable samples include extracts tissues such as brain, breast, ovary, lung, colon, pancreas, testes, liver, muscle and bone tissues or from neoplastic growths derived from such tissues.

The antibody isoform specific antagonist of PAK kinase may be bound to a solid support and/or packaged into kits in a suitable container along with suitable reagents, controls, instructions and the like.

Antagonists

The methods and compositions described here rely, in some embodiments, on blocking an activity of a PAK isoform, for example αPAK, βPAK or γPAK. Agents which are capable of decreasing or inhibiting the activity of the PAK isoform are referred to as antagonists of that activity.

The term “antagonist”, as used in the art, is generally taken to refer to a compound which binds to an enzyme and inhibits the activity of the enzyme. The term as used here, however, is intended to refer broadly to any agent which inhibits the activity of a molecule, not necessarily by binding to it. Accordingly, as used generally, it includes agents which affect the expression of a PAK isoform, for example αPAK, βPAK or γPAK, or the expression of modulators of the activity of the PAK isoform. The specific activity which is inhibited may be any activity which is characteristic of the enzyme or molecule, for example, as discussed above.

The antagonist may bind to and compete for one or more sites on the relevant molecule, for example, a PAK isoform, for example αPAK, βPAK or γPAK, preferably, a catalytic site or a binding site of the PAK isoform. As described above, the isoform specific antagonist of PAK kinase preferably interferes with, or prevents, the binding of Nck to the PAK isoform. Preferably, it competes for an Nck binding region of the PAK isoform.

Preferably, such binding blocks the interaction between the molecule and another entity (for example, the interaction between the PAK isoform and Nck). However, the antagonist need not necessarily bind directly to a catalytic or binding site, and may bind for example to an adjacent site, for example, an adjacent site in the PAK polypeptide, or even another protein (for example, a protein which is complexed with the enzyme) or other entity on or in the cell, so long as its binding reduces the activity of the enzyme or molecule or molecules in question.

Where antagonists of a enzyme such as a PAK enzyme are concerned, an antagonist may include a substrate of the enzyme, or a fragment of this which is capable of binding to the enzyme. An example is Nck. In addition, whole or fragments of a substrate generated natively or by peptide synthesis may be used to compete with the substrate for binding sites on the enzyme. Alternatively, or in addition, an immunoglobulin (for example, a monoclonal or polyclonal antibody) capable of binding to the PAK isoform, for example αPAK, βPAK or γPAK, may be used. The antagonist may also include a peptide or other small molecule which is capable of interfering with the binding interaction. Other examples of antagonists are set forth in greater detail below, and will also be apparent to the skilled person.

Blocking the activity of a PAK isoform may also be achieved by reducing the level of expression of the PAK isoform in the cell. For example, the cell may be treated with antisense compounds, for example oligonucleotides having sequences specific to the PAK isoform mRNA.

As used herein, in general, the term “antagonist” includes but is not limited to agents such as an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex.

The terms “antagonist” and “agent” are also intended to include, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, which bind to and occupy the active site of the polypeptide thereby making the catalytic site inaccessible to substrate such that normal biological activity is prevented, are also included. Examples of small molecules include but are not limited to small peptides or peptide-like molecules.

Rho GTPases

The Rho family of GTPases control growth and morphological cell signalling pathways downstream of various membrane-bound receptors (Hall, 1994,1998; Symons, 1996; Lim et al., 1996; Van Aeist and D'Souza-Schorey, 1997). Growth factors such as LPA, bradykinin and PDGF activate RhoA, Cdc42Hs and Rac1 to induce the formation of actin stress fibres, filopodia and lamellipodia (Ridley and Hall, 1992; Ridley et al., 1992; Kozma et al., 1995; Nobes and Hall, 1995). The activity of these G-proteins is co-ordinated. Cdc42Hs and Rac1 are directly coupled generating filopodia and lamellipodia in rapid succession (Kozma et al., 1995; Nobes et al., 1995). Both Cdc42Hs and Rac1 can induce the breakdown of stress fibres in Swiss 3T3 cells (Kozma et. al., 1995) and focal adhesions (Manser et. al., 1997). RhoA appears to antagonise and oppose the morphological changes induced by Cdc42Hs and Rac1 (Kozma et. al., 1995, 1996, 1997). For example, C3 toxin, the Rho inactivator, induces Cdc42Hs and Rac1-dependent neurite outgrowth in N1E-115 cells and primary neurons (Kozma et al., 1997; unpublished data) and filopodia formation in Swiss 3T3 cells (Hirose et al., 1998).

RhoA, Rac1 and Cdc42Hs induce the formation of the multimeric protein complexes that link integrin receptors to F-actin structures, the so-called ‘focal adhesions’ (FAs) or more generally ‘focal complexes’ (FCs; Ridley and Hall 1992; Ridley et al., 1992; Nobes et al., 1995). Typical components of FCs are vinculin, paxillin, integrins and tyrosine kinases such as FAK (for review see Llic et. al., 1997). In Swiss 3T3 cells three distinct FC types can be distinguished and each associates with a different F-actin structure; (i) arrowhead-stress fibres, (ii) oblong-filopodia and (iii) round-lamellipodia (Nobes and Hall, 1995, Kozma et. al., 1995). The mechanism by which RhoA, Rac1 and Cdc42Hs regulate the formation of FCs/FAs and associated F-actin microfilaments is under intense study.

Fragments, Homologues, Variant and Derivatives

It will be appreciated that fragments, homologues, variants and derivatives of the peptide isoform specific antagonists of PAK kinase may themselves have PAK isoform specific antagonist activity. We therefore disclose the use of such fragments, homologues, variants and derivatives in treatment and/or prevention of disease.

Homologues

In the context of this document, a “homologous” sequence is taken to include an amino acid sequence which is at least 15, 20, 25, 30, 40, 50, 60, 70, 80 or 90% identical, preferably at least 95 or 98% identical at the amino acid level over at least 10, 15, 20, 25, 30, 40, 50, 60, 70, 80, 90, 100, 110 or 115 amino acids with the target sequence, for example, a peptide isoform specific antagonist of PAK kinase. In particular, homology should typically be considered with respect to those regions of the sequence known to be essential for protein function rather than non-essential neighbouring sequences. This is especially important when considering homologous sequences from distantly related organisms.

Although homology can also be considered in terms of similarity (i.e. amino acid residues having similar chemical properties/functions), in the context of the present document it is preferred to express homology in terms of sequence identity.

Homology comparisons can be conducted by eye, or more usually, with the aid of readily available sequence comparison programs. These publicly and commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Advantageously, the BLAST algorithm is employed, with parameters set to default values. The BLAST algorithm is described in detail at the National Center for Biotechnology Information website, which is incorporated herein by reference. The search parameters are defined as follows, can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available at http://www.ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see the National Center for Biotechnology Information website). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at the National Center for Biotechnology Information website. In some embodiments, no gap penalties are used when determining sequence identity.

Once the software has produced an optimal alignment, it is possible to calculate % homology, preferably % sequence identity. The software typically does this as part of the sequence comparison and generates a numerical result.

Variants and Derivatives

The terms “variant” or “derivative” in relation to the amino acid sequences disclosed here includes any substitution of, variation of, modification of, replacement of, deletion of or addition of one (or more) amino acids from or to the sequence providing the resultant amino acid sequence retains substantially the same activity as the unmodified sequence. Preferably, the modified sequence has at least one biological activity as the unmodified sequence, preferably all the biological activities of the unmodified sequence. Preferably, the “variant” or “derivative” has at least one biological activity of the peptide isoform specific antagonist of PAK kinase, as described above, that is to say, the ability to specifically inhibit at least one activity of the relevant PAK kinase isoform.

Polypeptides having the amino acid sequence shown in the description and Examples, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the peptide isoform specific antagonist of PAK kinase. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides also include fragments of the peptide isoform specific antagonists of PAK kinase disclosed here. Preferably fragments comprise at least one epitope. Methods of identifying epitopes are well known in the art. Fragments will typically comprise at least 6 amino acids, more preferably at least 10, 20 or more amino acids.

Peptide isoform specific antagonists of PAK kinase, fragments, homologues, variants and derivatives, are typically made by recombinant means. However they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6xHis (SEQ ID NO: 64), GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins may also be obtained by purification of cell extracts from animal cells.

The peptide isoform specific antagonists of PAK kinase, variants, homologues, fragments and derivatives disclosed here may be in a substantially isolated form. It will be understood that such polypeptides may be mixed with carriers or diluents which will not interfere with the intended purpose of the protein and still be regarded as substantially isolated. They may also be in a substantially purified form, in which case it will generally comprise the protein in a preparation in which more than 90%, e.g. 95%, 98% or 99% of the protein in the preparation is a protein.

The peptide isoform specific antagonists of PAK kinase, variants, homologues, fragments and derivatives disclosed here may be labelled with a revealing label. The revealing label may be any suitable label which allows the polypeptide, etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols.

A peptide isoform specific antagonist of PAK kinase, variant, homologue, fragment or derivative disclosed here, optionally labelled, my also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The peptide isoform specific antagonist of PAK kinase, variants, homologues, fragments and derivatives disclosed here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which the peptide isoform specific antagonist of PAK kinase, variants, homologues, fragments and derivatives disclosed here are expressed may be used in assay systems to identify candidate substances which interfere with or enhance the functions of the polypeptides in the cell.

Pharmaceutical Compositions

While it is possible for the composition comprising the isoform specific antagonist of PAK kinase, for example, a peptide, antibody, or nucleic acid encoding these, or a small molecule, to be administered alone, it is preferable to formulate the active ingredient as a pharmaceutical formulation. We therefore also disclose pharmaceutical compositions comprising isoform specific antagonist of PAK kinase. Such pharmaceutical compositions are useful for delivery of isoform specific antagonist of PAK kinase to an individual for the treatment or alleviation of symptoms as described.

The composition may include the isoform specific antagonist of PAK kinase, a structurally related compound, or an acidic salt thereof. The pharmaceutical formulations comprise an effective amount of isoform specific antagonist of PAK kinase, together with one or more pharmaceutically-acceptable carriers. An “effective amount” of an isoform specific antagonist of PAK kinase is the amount sufficient to alleviate at least one symptom of a disease as described above.

The effective amount will vary depending upon the particular disease or syndrome to be treated or alleviated, as well as other factors including the age and weight of the patient, how advanced the disease etc state is, the general health of the patient, the severity of the symptoms, and whether the isoform specific antagonist of PAK kinase is being administered alone or in combination with other therapies.

Suitable pharmaceutically acceptable carriers are well known in the art and vary with the desired form and mode of administration of the pharmaceutical formulation. For example, they can include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The pharmaceutical compositions disclosed here include those suitable for topical and oral administration, with topical formulations being preferred where the tissue affected is primarily the skin or epidermis (for example, psoriasis, eczema and other epidermal diseases). The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated. As an example, an acute skin proliferation disease generally is treated with aqueous drying preparations, whereas chronic skin proliferation disease is treated with hydrating preparations. Soaks are the easiest method of drying acute moist eruptions. Lotions (powder in water suspension) and solutions (medications dissolved in a solvent) are ideal for hairy and intertriginous areas. Ointments or water-in-oil emulsions, are the most effective hydrating agents, appropriate for dry scaly eruptions, but are greasy and depending upon the site of the lesion sometimes undesirable. As appropriate, they can be applied in combination with a bandage, particularly when it is desirable to increase penetration of the agent composition into a lesion. Creams or oil-in-water emulsions and gels are absorbable and are the most cosmetically acceptable to the patient. (Guzzo et al, in Goodman & Gilman's Pharmacological Basis of Therapeutics, 9th Ed., p. 1593-15950 (1996)). Cream formulations generally include components such as petroleum, lanolin, polyethylene glycols, mineral oil, glycerin, isopropyl palmitate, glyceryl stearate, cetearyl alcohol, tocopheryl acetate, isopropyl myristate, lanolin alcohol, simethicone, carbomen, methylchlorisothiazolinone, methylisothiazolinone, cyclomethicone and hydroxypropyl methylcellulose, as well as mixtures thereof.

Other formulations for topical application include shampoos, soaps, shake lotions, and the like, particularly those formulated to leave a residue on the underlying skin, such as the scalp (Arndt et al, in Dermatology In General Medicine 2:2838 (1993)).

In general, the concentration of the isoform specific antagonist of PAK kinase composition in the topical formulation is in an amount of about 0.5 to 50% by weight of the composition, preferably about 1 to 30%, more preferably about 2-20%, and most preferably about 5-10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of active ingredient must penetrate a patient's skin in order to obtain a desired pharmacological effect. It is generally understood that the absorption of drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of drug between the stratum corneum and the vehicle and the diffusion coefficient of drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (J. Pharm. Sci., 60:1175-1179 (1971) demonstrated that in vivo efficacy of topically applied steroids was proportional to the steroid penetration rate into dermatomed human skin in vitro.

A skin penetration enhancer which is dermatologically acceptable and compatible with the agent can be incorporated into the formulation to increase the penetration of the active compound(s) from the skin surface into epidermal keratinocytes. A skin enhancer which increases the absorption of the active compound(s) into the skin reduces the amount of agent needed for an effective treatment and provides for a longer lasting effect of the formulation. Skin penetration enhancers are well known in the art. For example, dimethyl sulfoxide (U.S. Pat. No. 3,711,602); oleic acid, 1,2-butanediol surfactant (Cooper, J. Pharm. Sci., 73:1153-1156 (1984)); a combination of ethanol and oleic acid or oleyl alcohol (EP 267,617), 2-ethyl-1,3-hexanediol (WO 87/03490); decyl methyl sulphoxide and Azone.RTM. (Hadgraft, Eur. J. Drug. Metab. Pharmacokinet, 21:165-173 (1996)); alcohols, sulphoxides, fatty acids, esters, Azone.RTM., pyrrolidones, urea and polyoles (Kalbitz et al, Pharmazie, 51:619-637 (1996));

Terpenes such as 1,8-cineole, menthone, limonene and nerolidol (Yamane, J. Pharmacy & Pharmocology, 47:978-989 (1995)); Azone.RTM. and Transcutol (Harrison et al, Pharmaceutical Res. 13:542-546 (1996)); and oleic acid, polyethylene glycol and propylene glycol (Singh et al, Pharmazie, 51:741-744 (1996)) are known to improve skin penetration of an active ingredient.

Levels of penetration of an agent or composition can be determined by techniques known to those of skill in the art. For example, radiolabeling of the active compound, followed by measurement of the amount of radiolabeled compound absorbed by the skin enables one of skill in the art to determine levels of the composition absorbed using any of several methods of determining skin penetration of the test compound. Publications relating to skin penetration studies include Reinfenrath, W G and G S Hawkins. The Weaning Yorkshire Pig as an Animal Model for Measuring Percutaneous Penetration. In: Swine in Biomedical Research (M. E. Tumbleson, Ed.) Plenum, New York, 1986, and Hawkins, G. S. Methodology for the Execution of In Vitro Skin Penetration Determinations. In: Methods for Skin Absorption, B W Kemppainen and W G Reifenrath, Eds., CRC Press, Boca Raton, 1990, pp. 67-80; and W. G. Reifenrath, Cosmetics & Toiletries, 110:3-9 (1995).

For some applications, it is preferable to administer a long acting form of agent or composition using formulations known in the arts, such as polymers. The agent can be incorporated into a dermal patch (Junginger, H. E., in Acta Pharmaceutica Nordica 4:117 (1992); Thacharodi et al, in Biomaterials 16:145-148 (1995); Niedner R., in Hautarzt 39:761-766 (1988)) or a bandage according to methods known in the arts, to increase the efficiency of delivery of the drug to the areas to be treated.

Optionally, the topical formulations can have additional excipients for example; preservatives such as methylparaben, benzyl alcohol, sorbic acid or quaternary ammonium compound; stabilizers such as EDTA, antioxidants such as butylated hydroxytoluene or butylated hydroxanisole, and buffers such as citrate and phosphate.

The pharmaceutical composition can be administered in an oral formulation in the form of tablets, capsules or solutions. An effective amount of the oral formulation is administered to patients 1 to 3 times daily until the symptoms of the disease alleviated. The effective amount of agent depends on the age, weight and condition of a patient. In general, the daily oral dose of agent is less than 1200 mg, and more than 100 mg. The preferred daily oral dose is about 300-600 mg. Oral formulations are conveniently presented in a unit dosage form and may be prepared by any method known in the art of pharmacy. The composition may be formulated together with a suitable pharmaceutically acceptable carrier into any desired dosage form. Typical unit dosage forms include tablets, pills, powders, solutions, suspensions, emulsions, granules, capsules, suppositories. In general, the formulations are prepared by uniformly and intimately bringing into association the agent composition with liquid carriers or finely divided solid carriers or both, and as necessary, shaping the product. The active ingredient can be incorporated into a variety of basic materials in the form of a liquid, powder, tablets or capsules to give an effective amount of active ingredient to treat the disease.

Other therapeutic agents suitable for use herein are any compatible drugs that are effective for the intended purpose, or drugs that are complementary to the agent formulation. The formulation utilized in a combination therapy may be administered simultaneously, or sequentially with other treatment, such that a combined effect is achieved.

The invention is described further, for the purpose of illustration only, in the following examples.

### EXAMPLES

### Example 1

### Materials and Methods

Materials

The chondroitin sulphate proteogylcan (CSPG) is from Chemicon. The peptide delivery system is BioPorter by Gene Therapy Systems and supplied by Q-Biogene, UK. Laminin is from life Technologies and as is all the tissue culture reagents. Rat IgG used to confirm positively injected cells is from Pierce Bioscience. TRITC dye used to mark the laminin is from Sigma. Peptides and antibodies are from Santa Cruz apart from Vinculin antibody which is from Sigma. Laminin antibody is from Polyscience. All other general reagents are from Sigma or Merck.

Cell Culture

Swiss 3T3 and N1E-115 cells are grown as described previously (Kozma et al., 1995, 1997). Bac1.2F5 cells a macrophage cell line subcloned to be CSF-dependent for growth (Allen et al., 1997). Bac1 cells are grown in DMEM containing 10% foetal calf serum (FCS), glutamine, asparagine, β-mercaptoethanol (all from Sigma) with antibiotic and antimycotic agents (Gibco). CSF (a gift from the Genetics Institute) is added to final concentration 1.32 nM. Cells are grown at 37° C. with 5% CO₂ and passaged by trypsination/scraping and diluted to between 1:4 to 1:8.

Micromanipulation

Proteins purified from E. coli and C3 toxin are microinjected in 50 MM Tris pH 7.5, 50 mM NaCl, 5 mM MgCl₂ using an Eppendorf microinjector and Axiovert microscope. Successful injection is determined visually at the time of injection and by use of IgG. Cell viability is estimated to be greater 90%. Needles containing factors (CSF, LPA or buffer) are placed near particular cells to obtain gradients (Kozma et al., 1997).

Recombinant Proteins

Rho family GTPases are expressed as GST fusions using the pGEX system for expression as previously described (Ahmed et al., 1994, 1995). Briefly, a single colony is picked from freshly transformed cells and grown overnight in 100 ml of LB. Overnight cultures are diluted 1:100 and grown to an OD650 of 0.5 an which point IPTG to 1 mM is added and cells grown for a further 2 h. Cells are harvested, washed in 50 mM Tris pH 7.5/2.5 mM MgCl₂ and resuspended in the same buffer with 0.1% T X-100 and protease inhibitors after 100-fold concentration. To purify proteins cells are sonicated (4×30 sec.), cell debris discarded and the supernatant loaded onto glutathione-Sepharose columns. Proteins are eluted in 5 mM glutathione in 50 mM Tris pH 7.5/50 mM NaCl/2.5 mM CaCl₂ and then dialysed against the same buffer without glutathione overnight. Proteins are frozen at approximately 1-2 mg/ml at 70° C. in aliquots of 50-100 μL.

Peptides

Peptides were made using standard solid phase procedures on an Applied Biosystems peptide synthesiser.

Microscopy

Time lapse microscopy is carried out on a heated stage (37° C.) in an atmosphere of humidified air and 5% CO2. Cells are video recorded using a, Axiovert 135 microscope, Palmix TM-6CN video camera and Sony-u-matic V0-5800 PS video recorder. Individual images are captured using Bio-rad COMOS software and manipulated with Adobe photoshop. Confocal microscopy is carried out using a LSM 410 Zeiss scanning microscope with 3 single line lasers at 488 nM (argon), 543 nM and 633 nM, (HeNe).

In Situ Localisation

For in situ analysis primary/secondary antibodies are used at 1:100 dilution. PAK antibodies, all from Santa-Cruz, are as follows; αPAK N-20 (raised to rat amino acids 2-21), αPAK C-19 (raised to rat amino acids 525-544), βPAK (raised to mouse amino acids 2-20), γPAK (raised to human amino acids 2-20) and γPAK (raised to human amino acids 2-20). Vinculin, paxillin, phosphotyrosine are from Sigma. Integrin antibodies β1-integrin and α6-integrin a gift from A. Sonneberg, NCI, Netherlands. Bac1 cells are seeded onto coverslips and then grown for 24 h before the media is replaced with either CSF free, serum free, or CSF/serum free.

After manipulations, media is removed and coverslips rinsed with (×2) CB buffer (10 mM Mes pH 6.1, 150 mM NaCl, 5 mM EGTA, 5 mM MgCl₂, 5 mM glucose). Cells are then fixed with 4% paraformaldehyde in CB for 10 min at room temperature. Cells are then washed CB for 2×10 min. Cells are then permeabilised for 1 min in 0.5% T X-100 in CB, washed in CB for 2×10 min. Coverslips are then inverted onto 20 μl of blocking solution (3% BSA, TBS, 20 mM Tris pH, 0.154 mM NaCl, 2 mM EGTA, 2 mM MgCl₂).

Primary antibodies diluted 1/100 in 1% BSA in TBS are then exposed to coverslips and incubated at 37° C. for 2 h. Cells on coverslips are then washed in TBS 2×10 min. Secondary antibodies diluted in 1% BSA in TBS are incubated with coverslips for 1 h in a dark humid chamber. Finally, coverslips are washed 2×10 min in TBS and mounted in Immuno-Flore (ICN Flow). F-actin is stained using rhodamine conjugated phalloidin (0.1 μg/ml, Sigma). The slides are allowed to set overnight before viewing in a Zeiss axiovert microscope and Zeiss LSM 410 confocal.

Microinjection of N1E-115 Cells

Cells are plated out at 1.0×10⁵ cells in 1 ml in a 35 mm dish containing 2 laminin coated coverslips. The coverslips are first marked with a grid using a fine permanent marker and then coated with laminin and incubated overnight in DMEM, 10% FCS, 1% AB/AM. The media is replaced with serum free media (DMEM, 1% AB/AM) and the cells starved for 1 hour so that they flattened and are easier to inject. The peptides are injected at 10 μg/ml in PBS, Rat IgG at 1 mg/ml is added to act as a marker for injected cells.

The cells are injected using an Eppendorf microinjection apparatus on an Axioplan inverted microscope equipped with a heated stage maintained at 37° C. and a 5% CO₂ chamber. Humidity is maintained by a dish of sterile water inside the CO₂ chamber. Cells in a grid square are injected and positive injection checked visually at the time of injection or after fixation and staining for the injected marker. After injection the cells are returned to the incubator for a further 2 hours. Giving a total time without serum of 4 hours.

Cell Guidance Assays

(a) Coverslips

The coverslips are washed and sterilised and then coated in one of the following ways.

(i) Permissive/Attractive Boundary

The coverslips are covered with a solution of Poly-D-lysine at 10 μg/ml, and incubated for 1 hour at RT, then rinsed once with sterile H₂O and then allowed to air dry in the laminar flow hood before a drop of laminin 10 μg/ml or fibronectin 10 μg/ml is placed centrally and the coverslips incubated for 1 hour RT. The coverslips are then rinsed twice in sterile H₂O before drying in the hood. (ii) Control boundary. The coverslips are coated with laminin 10 μg/ml for 1 hour at RT then rinsed in sterile water and left to dry.

A second layer of laminin is applied as a drop mixed with 1 μg/ml TRITC label. Coverslips are left for 1 hour at RT and then rinsed twice with sterile water before allowing to dry before use. (iii) Attractive/repulsive boundary. Slides are coated with CSPG 5 μg/ml for 1 hour at 37° C. in a humid chamber then rinsed once in sterile H₂O and allowed to air dry before the drop of laminin at 10 μg/ml is applied to the centre of the coverslip and left for 1 hour at RT before rinsing twice in sterile water and allowing to dry before use. This coating is also done in reverse with the laminin applied first and then the tenascin or the CSPG applied second.

(b) Assay

The cells are then seeded onto the prepared coverslips at 2×10⁵ cells in 1 ml in a 35 mm petri dish, in DMEM, 10% FCS, and 1% AB/AM. Cells are allowed to attach overnight. The media is then removed and the coverslips rinsed in serum free media and incubated overnight (18 h) in serum free media. After the starvation the cells are fixed and stained to show the morphology of the cells at the boundary. Only neurites expressing neurofilamant are scored.

Preparation of BioPorter Reagent.

BioPorter regent is supplied as a dried powder and must be dissolved in a solvent before use. To each tube of BioPorter reagent 250 μl of methanol is added and the powder is re-suspended by vortexing for 10-20 seconds. 10 μl of the mix is transferred into new eppendorf tubes; the methanol is allowed to evaporate for 2 hours in a laminar flow hood. The dried reagent is stored at −20° C.

Protein Delivery System

Cells are plated out at 1.5×10⁵ cells in a 35 mm dish containing 2 laminin-coated coverslips and incubated overnight. The protein or peptide of interest is diluted in 100 μl PBS as to give a final concentration of 10 μg/ml and 1 μl of the FITC labelled goat IgG is added to act as a marker. This mix is added directly to the tube containing the BioPorter reagent is mixed by pipetting up and down 2-3 times and then allowed to stand for 5 minutes.

After 5 minutes, the cells are washed once with serum free DMEM media and then 1 ml of serum free DMEM is added to the tube containing the peptide and BioPorter mix it is vortexed gently before being added to the cells. The cells are incubated for 4 hours at 37° C. After the 4 hour incubation the cells are then incubated for a further 2 hours in 5% FCS DMEM or for cells undergoing a substrate guidance assay the 2 hour incubation is in serum free DMEM. The cells are fixed and stained.

“Far-Western” Analysis

Cdc42Hs and Rac1 probes are prepared by incubating purified proteins (1-5 μg) with 1 μl of [γ³²P]GTP (NEN; 6000 Ci/mmol, 10 mCi/ml; 1.6 μM) in 50 μl exchange buffer (50 mM NaCl, 25 mM Mes pH 6.5, 25 mM Tris-HCl pH 7.5, 1.25 mM EDTA, 1.25 mg/ml BSA, 1.25 mM DTT) for 10 min at room temperature.

Nitrocellulose filters with immobilised proteins (transfer by semi-dry blotting as described below) are then incubated 5 min at room temperature with Cdc42Hs/Rac1 probes in binding buffer (50 mM NaCl, 25 mM Mes pH 6.5, 25 mM Tris-HCl pH 7.5, 1.25 mM MgCl₂, 1.25 mg/ml BSA, 1.25 mM DTT, 0.5 mM GTP). After washing (×6) in 50 ml 50 mM NaCl/25 mM Mes pH 6.0/5 mM MgCl₂/0.25% T X-100 Cdc42Hs[γ³²P]GTP and Rac1[γ³²P]GTP binding activity is determined by exposure of nitrocellulose filters to X-ray film.

Protein Purification

Neutrophils are prepared as in (Grogan et al., 1997) and cytosol which contained the bulk of the PAKs made to 20 mg/ml in the presence of protease inhibitors; aprotinin, pepstatin, leupeptin, PMSF and TLK, at 1 μg/ml. Neutrophil cytosol is the applied to a 200 ml fast flow Q-sepharose column equilibrated with Pipes buffer (10 mM Pipes, pH 7.0, 100 mM KCl, 3 mM NaCl, 4 mM MgCl₂, in the presence of the above protease inhibitors.

The column is then washed with 400 ml Pipes buffer. Proteins are eluted with a 0-1 M NaCl gradient in Pipes buffer and fractions of 10 ml collected. 5 μl of each fraction is dot blotted onto nitrocellulose and a “Far-Western” analysis carried out with Cdc42Hs and Rac1 [γ³²P]GTP-labelled probes. Fractions 16-26 (0.25-0.45 M NaCl) containing the majority of the Cdc42Hs binding activity are pooled and dialysed overnight at 4° C. in Pipes buffer with two changes in buffer. Pooled fractions (100 ml; 5 mg/ml) are then applied to a 25 ml Heparin agarose column equilibrated in Pipes buffer.

The column is washed in 25 ml Pipes buffer. Proteins are eluted with a step gradient 0.1-0.5 M NaCl and 20 ml fractions collected. Fractions are then analysed by “Far-Western” analysis for Cdc42Hs binding activity. Fractions between 0.1-0.2 M NaCl are pooled and dialysed against phosphate buffer overnight. 20 ml of protein at 0.85 mg/ml is adjusted to 1.2 M ammonium sulphate and applied to a Phenyl sepharose column. The column is eluted with a 10 ml step gradient of 1.2-0.2 M ammonium sulphate. The column is then washed (×6) with 10 ml of 100 mM phosphate buffer.

Fractions 1-4 of the no salt elution containing the bulk of the Cdc42Hs binding activity are dialysed against 50 mM Tris pH 7.5, 150 mM NaCl, 5 mM MgCl₂, 2.5 mM CaCl₂, and 1 mM DTT and then applied to a Cdc42Hs affinity column which is prepared as described previously (Manser et al., 1994). Briefly, 1 ml of Cdc42Hs-GST fusion protein is loaded with GTP (see “Far-Western” analysis) and 2.5 ml of non-salt elution 1 applied to the column and allowed to interact for 1 h.

The Cdc42Hs column is then washed with (×6) 10 ml of 25 mM Mes pH 6.0, 100 mM NaCl, 0.5 mM MgCl₂, 0.05% Triton-X 100. The bound fraction is eluted with lamelli sample buffer (×2) buffer and run out on SDS-PAGE and the gel stained with Coomassie blue. Protein concentrations are determined by the method of Bradford. For protein sequencing bands at 62, 66 and 68 kDa are cut out, peptides isolated and sequenced.

Immunoblot Analysis

Proteins are run out on 10% SDS-PAGE for 50 min at 180V and then placed in blotting buffer (48 mM Tris, 39 mM glycine, 3 mM SDS) with 20% methanol and left on a shaker for 10 min. Gels are then blotted onto nitrocellulose filters using a Bio-rad semi-dry blotter. Filters are blocked in 5% milk powder (Marvel)/PBS/0.1% Tween for 1 h at room temperature. Filters are then washed in 50 ml of PBST in a 50 ml falcon tube.

After washing, filters are incubated in primary antibody in PBS/0.1% Tween/1% milk on a roller mixer for 60 min at room temperature. The primary antibody is then removed by washing in 50 ml PBST for 5 min (×5) and secondary antibody added. After incubation for 1 h filters are washed in PBST. Filters are blot dried and then developed using ECL reagents (Amersham Pharmacia Biotech) and exposed to Hyperfilm ECL (Amersham Pharmacia Biotech) for 1-30 min. For immunoblots primary/secondary antibodies are used at 1:1000 dilution.

Identification of PAK Isoforms in Neutrophils

PAK proteins are the major Cdc42Hs/Rac1 binding proteins in neutrophil cytosol as detected by overlay assays (Prigmore et al., 1995; Martin et al., 1995). To characterise the PAKs present in neutrophils we used Ste20 antibodies and a “Far-Western” analysis with Cdc42Hs and Rac1 (Q61L)-γ³²P[GTP] probes. Neutrophil cytosol is purified through four chromatographic columns: Q-Sepharose, Heparin agarose, Phenyl-Sepharose and Cdc42Hs-affinity (as described in detail in the Materials and Method section).

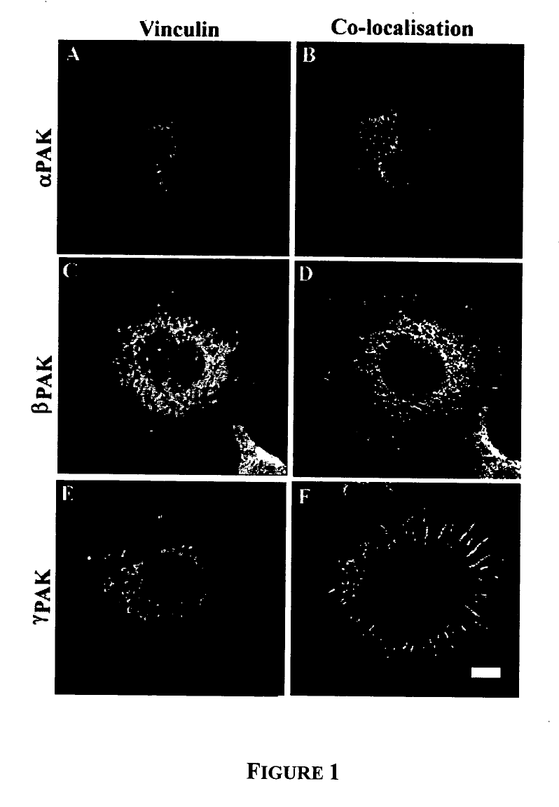

Starting material for the Cdc42Hs-affinity column is approximately 30% pure and using Ste20 antibodies three distinct PAK bands in the molecular mass range 62-66 kDa are visualised (FIG. 1A). Cdc42Hs and Rac1 probes also detected these bands and in addition signals above and below the Ste20 signals are present. With the Cdc42Hs probe in particular, which detected the bands more strongly than Rac1, two bands at 66-68 kDa could be seen.

To confirm that the proteins detected by anti-Ste20 antibodies and Cdc42Hs/Rac1 probes are indeed PAK proteins bands are excised from a gel after Cdc42Hs-affinity chromatography and the derived peptides sequenced. The band running at approximately 62 kDa is confirmed as γPAK (FYDSNTVK (SEQ ID NO: 60) matches residues 129-136 of γPAK). Sequences of bands excised at 66 and 68 kDa are PAKs but from these peptides it is not possible to conclude which PAK isoforms are present (e.g. GTEAPAVVTEEE (SEQ ID NO: 61) and SVIDPVPAPV (SEQ ID NO: 62)). Additionally, a strong Coomassie blue band running at approximately 70 kDa, which is not detected with anti-Ste20 antibodies or Cdc42Hs/Rac1 probes, is found (FIG. 1A, upper arrow lane 5). Peptide sequencing of this 70 kDa band revealed that it is Annexin VI (data not shown).

The molecular masses 62, 66 and 68 kDa of the PAK bands seen with Cdc42Hs/Rac and Ste20 probes suggested that the three mammalian PAKs α, β and γ (Manser and Lim, 1999) are present in this purified material from neutrophil cytosol. The affinity purified PAK fraction is examined further with isoform specific antibodies (raised to peptides from the N-terminal of the different PAK isoforms, see Material and Methods section).

Apart from αPAK C19, the other antibodies detected specific bands, γ-62 kDa, β-65 kDa and α-64/68 kDa. Overlay of the α, β, and γ autoradiographs showed that the 62, 65, 64/68 kDa bands are distinct (FIG. 1B). The combined signals obtained with α, β and γ (or h) antibodies is similar to the signal obtained with the C19 antibody, although C19 did have preference for the 66-68 kDa bands. Thus this latter antibody is non-specific. Antibody signals are blocked by the immunising peptides but not by control peptides. Taken together, these results shows that at least three different PAK isoforms are expressed in neutrophils and that the peptide antibodies can detect specifically PAK α, β and γ isoforms.

PAK Isoforms in Bac1 Macrophages and N1E-115 neuroblastoma To investigate the in situ localisation of endogenous PAK α, β and γ isoforms the model macrophage cell line Bac1.2F5 is used. As shown in FIG. 1C, PAK α, β, and γ are well expressed in Bac1 macrophages. The signals obtained with the αPAK antibody suggest the presence of phosphorylated forms of this protein or related isoforms. Similar results are obtained with neutrophils.

### Example 2

### PAK Isoforms Localise to Distinct Focal Complexes (FCs)

Isoform-specific anti-Pak antibodies are used to localize endogenous protein. The specificity of the antibodies is confirmed using extracts from cell lines and purified neutrophil PAK isoform proteins (see Example 1 above).

We screened a range of cell lines and found Bac1 cells to express high levels of PAK isoforms α, β and γ, making them ideal for localization studies. Initially, we examined freshly plated spreading cells as these would be more likely to possess morphological structures important for cell motility such as, filopodia, lamellipodia and membrane ruffles.

In situ staining with vinculin antibodies reveals three major and distinct types of FCs in spreading cells; rib-like structures radiating out to the leading edge, round complexes near the leading edge and smaller round complexes in the centre of the cell. Ribs are thought to be stabilised filopodia that trail the leading edge (being consumed during lamellipodial expansion). Time lapse phase-contrast analysis of Bac1 cells reveals that membrane ruffling occurred from the perinuclear region to the periphery of the cell (data not shown).

In situ double staining of spreading cells with PAK isoform-specific antibodies and vinculin antibodies revealed distinct colocalisation patterns. αPAK is colocalised with vinculin in the centre of the cell (FIG. 1, panels A and B), γPAK at the periphery and in particular with the rib-like structures and filopodia (FIG. 1, panels E and F) while βPAK colocalised with vinculin predominantly in the peri-nuclear region with membrane ruffles and at the periphery with lamellipodia (FIG. 1, panels C and D).

Interestingly, αPAK is never found at the periphery of spreading cells or in areas of membrane ruffling (FIG. 1). The αPAK complexes stained positive for a number of typical FC components including; paxillin, α1 and β6 integrins, and phosphotyrosine. Taken together, these results suggest that γPAK and βPAK are associated with FCs involved in cell extension while αPAK is associated with FCs involved in cell adhesion. Similar PAK isoform colocalisation results are obtained in growing cells (data not shown).

### Example 3

### Cdc42Hs/Rac1 Activation can Antagonize RhoA Function in Bac1 Cells

To establish whether αPAK complexes require RhoA activity the specific inhibitor C3 toxin is employed. A complication with the use of C3 is that in N1E-115 and Swiss 3T3 cells it leads to activation Cdc42Hs and Rac1 (Kozma et al., 1997; Hirose et al., 1998). In these cell types an antagonistic relationship between Cdc42Hs/Rac1 and RhoA exists (Kozma et al., 1995, 1997). Thus, it is important to establish the relationship between Cdc42Hs, Rac1 and RhoA in Bac1 macrophages so that the effects of C3 toxin can be interpreted.

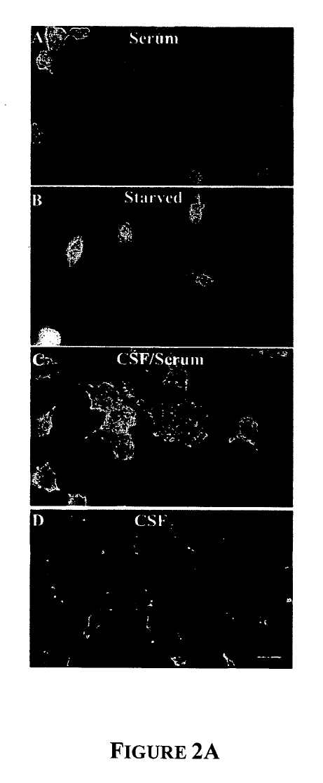

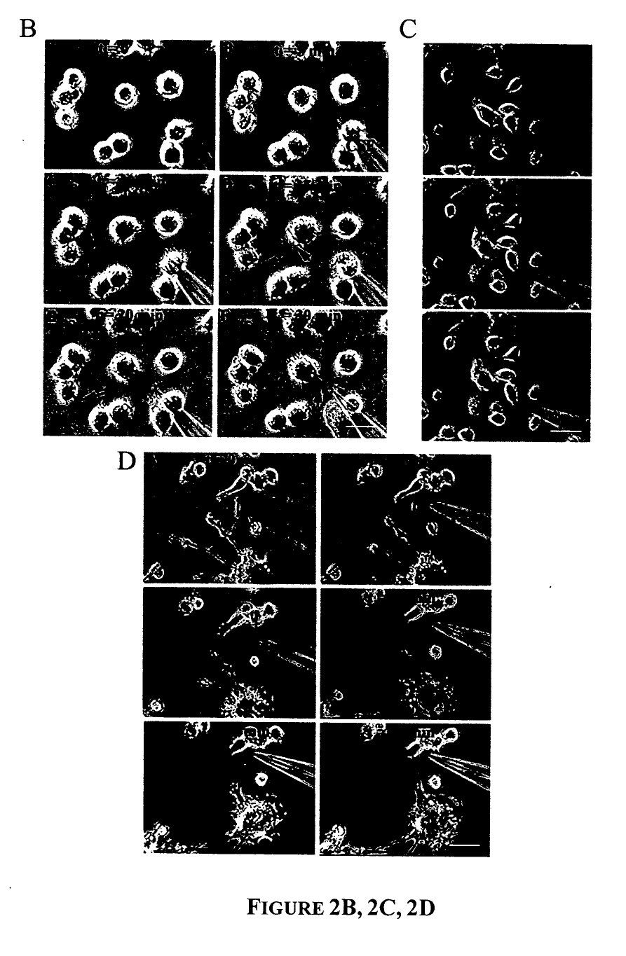

As a first step in understanding the relationship between Cdc42Hs/Rac1 and RhoA we compare growing Bac1 cells (FIG. 2A, panel C) with factor starved cells and looked at the effect of these treatments on cell morphology and F-actin structures. Cells starved of serum for 24 h underwent a differentiation process where long neurite-like processes are extended and cells stopped growing (FIG. 2A, panel D). Cells starved of CSF rounded up (FIG. 2A, panel A) and cells starved of both serum and CSF became elongated (FIG. 2A, panel B). Since CSF activates Cdc42Hs and Rac1 (Allen et al., 1997) and serum (LPA) activates RhoA these results suggest that the former two GTPases drive cell extension and the latter GTPase induces cell rounding, possibly by increasing levels of cell adhesion.

To investigate the effects of Rho family GTPases further we carry out a phase-contrast time lapse analysis of Bac1 cells responding to gradients of CSF and LPA (FIG. 2B-D). CSF induces the formation of filopodia and lamellipodia (FIG. 2B) and over the time course of the experiment (30 min) cells are seen moving towards the source of CSF (needle). In contrast, the serum component LPA induces cell rounding and collapse of processes (FIG. 2C).

Thus it appears that, as in other cell types, Cdc42Hs/Rac1 promote cell extension while RhoA promotes cell rounding (Kozma et al., 1997; Sarner et al., 2000). To examine whether CSF protects cells from LPA mediated cell rounding, the experiment shown in FIG. 2D is carried out. CSF is released via a needle to a Bac1 cell possessing processes. After 1.5 min LPA is added to the medium. In the absence of CSF, LPA causes cell rounding. FIG. 2D shows that CSF can protect the process nearest to the source of CSF from LPA mediated process collapse. In addition, cells near the source of CSF can be seen to put out filopodia (FIG. 2D) and the surface of the cell with processes nearest to the CSF begins to ruffle and move closer to the CSF.

C3 toxin microinjection into Bac1 cells induces the rapid induction of filopodia and this is followed by lamellipodia formation and membrane ruffling (under time-lapse phase contrast microscopy, data not shown). Process formation can be seen to occur 3 h after C3 microinjection followed by morphologically differentiation at 24 h. Co-injection of dominant negative Cdc42Hs or Rac1 with C3 inhibited the formation of filopodia and lamellipodia, respectively (data not shown). These results show that, as in Swiss 3T3 cells and N1E-115 cells, Cdc42Hs and Rac1 can antagonise RhoA function and vice versa (FIG. 2E).

### Example 4

### αPAK Localises to RhoA-Dependent FCs

From the analysis presented above (FIG. 2), if the αPAK containing complexes are dependant on RhoA activity, the effects of C3 toxin should be to cause a redistribution of FCs from the cell center to the periphery.

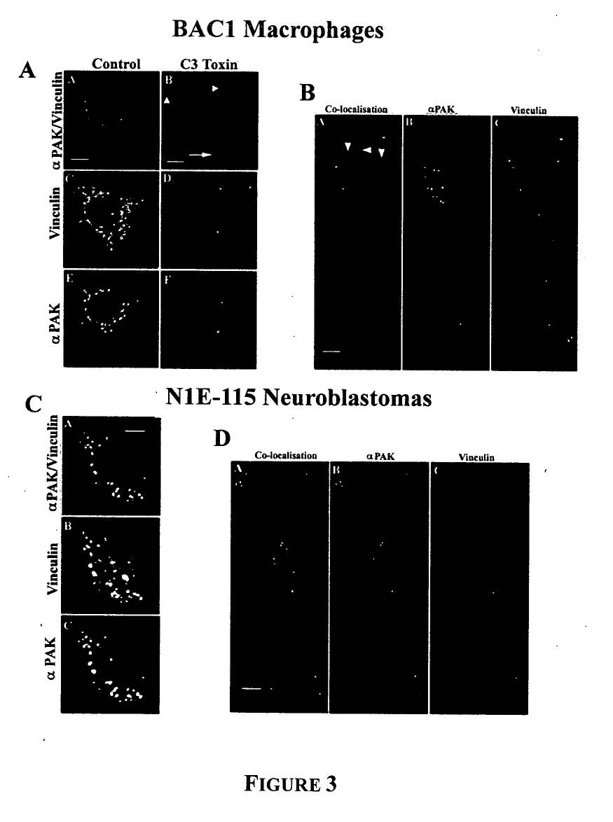

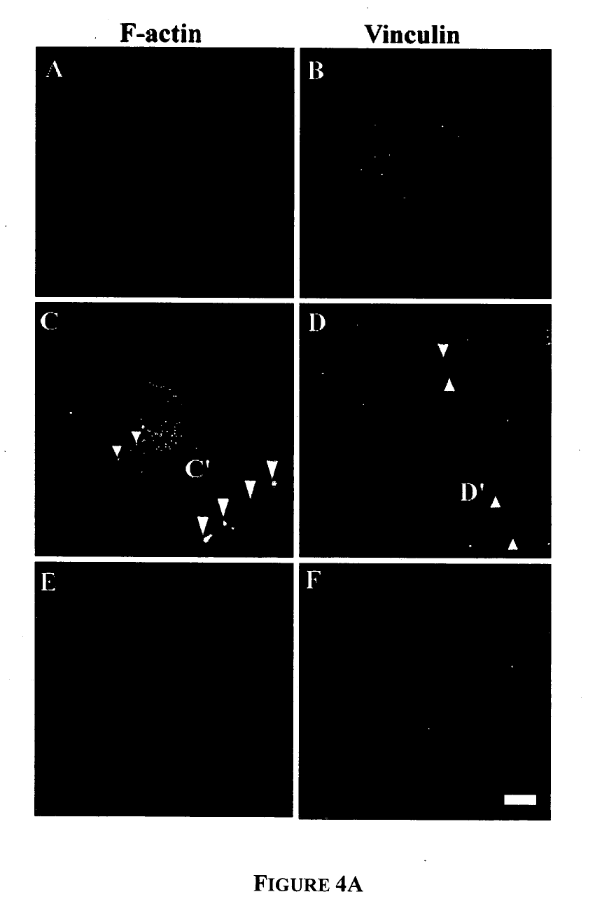

FIG. 3A shows an experiment where a growing cell is microinjected with C3 toxin and then double stained for αPAK and vinculin. The C3 microinjected cell is flattened, the FCs in the centre of the cell have dispersed and prominent FCs at the cell periphery that do not colocalise with αPAK are formed. The colocalisation of αPAK with vinculin in the cell body is significantly reduced by C3 toxin microinjection (FIG. 3A, compare panels A and B).

Next we examine the changes that occur to αPAK/vinculin colocalisation in Bac1 and N1E-115 cells that have undergone differentiation (FIG. 3). Both cell types show that the size and number of αPAK containing FCs decreases with differentiation and peripheral αPAK non-staining FCs are formed (FIG. 3B and D, panels A). This loss of αPAK/vinculin complexes correlates with a switch from RhoA dominance during cell growth to Cdc42Hs/Rac1 dominance during cell differentiation.

### Example 5

### αPAK Localises to Dynamic FAs and Stress Fibres in Swiss 3T3 Cells

The data presented above suggests that αPAK localises to RhoA-dependent FCs in Bac1 and N1E-115 cells. Swiss 3T3 cells possess the classical arrowhead shaped FAs that require RhoA activity for their formation (Hall, 1998). As shown previously (Dharmawardhane et al., 1997), we found that αPAK did not colocalise with vinculin in FAs in growing Swiss 3T3 cells (data not shown). The absence of αPAK in FAs in Swiss 3T3 cells could be due to the relative stability of these complexes compared to αPAK containing FCs in Bac1 and N1E-115 cells.

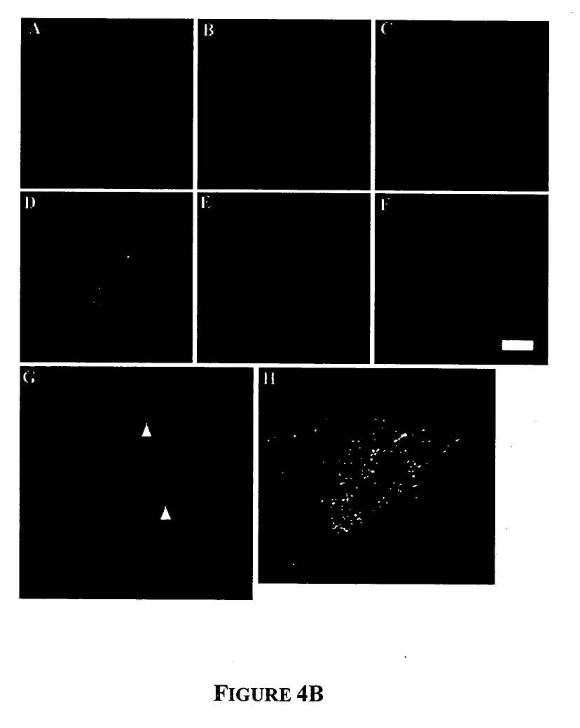

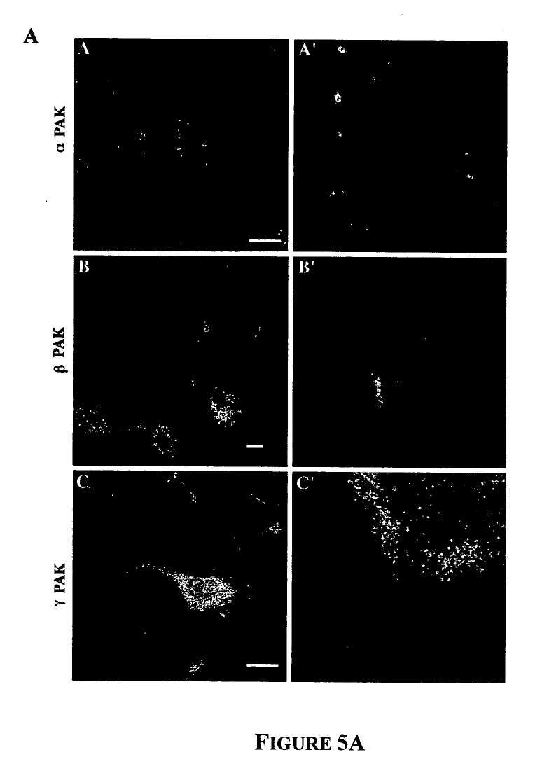

To address this possibility we utilise freshly plated Swiss 3T3 cells because these cells have more dynamic FCs and FAs than growing cells. Freshly plated Swiss 3T3 cells undergo a sequence of morphological events including filopodia and lamellipodia formation and membrane ruffling that lead to the formation of FAs. Cells are followed for 24 h after plating with staining for αPAK, F-actin and vinculin. At 1 h after plating cells are round with circular whorls of F-actin and cytoplasmic F-actin clusters (FIG. 4A, panel A). Vinculin staining is present on the periphery and in the clusters (FIG. 4A, panel B).

At this stage αPAK colocalises with F-actin and vinculin in clusters (FIG. 4A panels A and B). At 4 h after plating of Swiss 3T3 cells αPAK colocalises with the newly forming stress fibres and FAs (FIG. 4A, panels C and D and insets c′ and d′). By 24 h after plating αPAK does not colocalise significantly with either stress fibres or FAs which now appear stable and well formed. (FIG. 4A, panels E and F). During the 24 h time course of these experiments αPAK is never seen at the periphery with either filopodia or lamellipodia.

FIG. 4B shows a Z-series (panel A-bottom, to panel F-top) from a confocal microscope analysis of a spreading Swiss 3T3 cell double stained with αPAK and F-actin. Stress fibres are seen forming and this cell also contains F-actin clusters and a ruffling edge. αPAK colocalises with F-actin in both the clusters (FIG. 4B, panel D) and stress fibres (FIG. 4B, panel G) but not the ruffling edge (FIG. 4B, panels C and D). αPAK is present in large aggregates in the centre of the cell (FIG. 4B, panel H) that disperse once cells have become well spread>24 h.

The experiments shown in FIG. 4 follow the formation of stress fibres and FAs. To examine events during the breakdown of stress fibres and FAs serum starved Swiss 3T3 cells are treated with bradykinin and double stained for αPAK and F-actin/vinculin. αPAK is recruited to both stress fibres and FAs during the process of disassembly (data not shown). After stress fibres and FAs have disappeared αPAK colocalises in F-actin clusters with vinculin (data not shown). Thus αPAK in Swiss 3T3 cells is present in dynamic but not stable FAs and stress fibres.

### Example 6

### PAK Isoforms Localize to Distinct F-Actin Structures

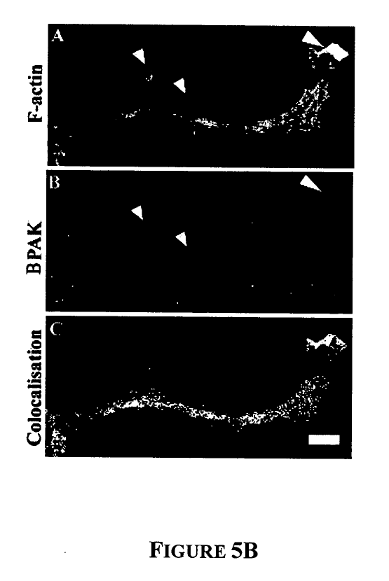

To determine whether PAK isoforms colocalise with F-actin structures differentiated Bac1 and N1E-115 cells are examined. Differentiated cells are chosen as they possess distinct F-actin structures in contrast to the heterogeneous and relatively small growing cells. Bac1 cells that have been starved of CSF and serum (FIG. 5) or of serum alone (data not shown) are double stained with PAK isoform specific antibodies and phalloidin. As shown in FIG. 2 CSF/serum starvation causes Bac1 cells to elongate while starvation of serum alone induces the formation of long processes.

In contrast to spreading cells, cells undergoing elongation and process outgrowth undergo intense membrane ruffling and form more robust filopodia at the leading edge (FIG. 5). The three PAK isoforms localise with distinct F-actin structures. αPAK is found in the centre of the cell with small round F-actin clusters which are more numerous in elongating cells (FIG. 5A, panels A/A′) than in fully differentiated cells. αPAK colocalises with F-actin in the centre/nuclear region of the cell but not at the cell periphery (FIG. 5A, panels A/A′). βPAK is found prominently in membrane ruffles and with lamellipodia but not in filopodia (FIG. 5A, panels B/B′; FIG. 5B). γPAK colocalises with F-actin most obviously in the filopodia and along the length of the processes (FIG. 5A, panel C/C′). γPAK is weakly associated with areas of membrane ruffling.

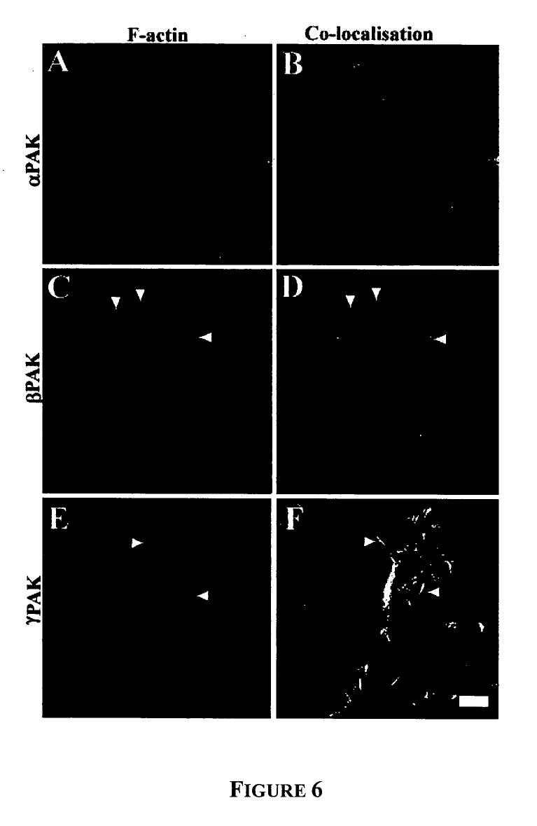

To confirm that the localisation of PAK isoforms to distinct F-actin structures is not unique to Bac1 cells we carry out in situ staining experiments with serum starved N1E-115 (FIG. 6). As with Bac1 and Swiss 3T3 cells, αPAK colocalises with small F-actin clusters but did not colocalise with F-actin in filopodia or lamellipodia. βPAK colocalises with lamellipodia primarily while γPAK colocalises with filopodia (FIG. 6).

### Example 7

### N-Terminal Peptides Act as PAK Isoform-Specific Inhibitors

We have found through analysis of PAK isoform primary sequences that the N-terminal end (amino acid residues 1-25, P1), which incorporates the core 13 amino acid residue Nck binding site, represents the most divergent part of the proteins We have therefore designed peptides from these sequences for use as potential targets for isoform-specific inhibitors.

Although not wishing to be bound by any particular theory, we believe that peptides from this region serve as isoform-specific inhibitors of PAK function by preventing interaction with Nck (or other potential targets).

αP1 and αP1*

Initially, we used two 13 mer peptides from the N-terminal of αPAK in combination: EDKPPAPPMRNTSMI (αP1) (SEQ ID NO: 1) and EDKPPAPPMRNTS*MI (αP1*) (SEQ ID NO: 63).

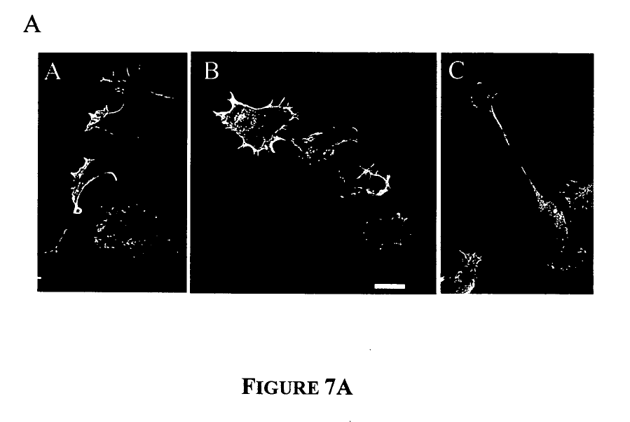

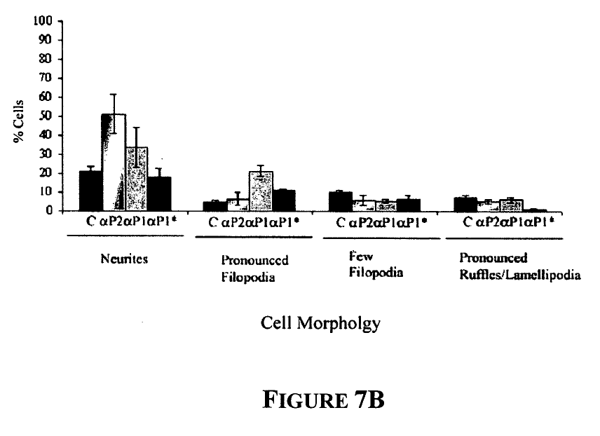

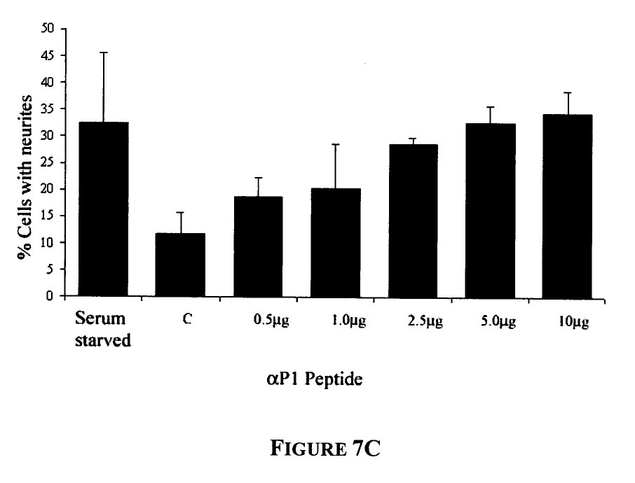

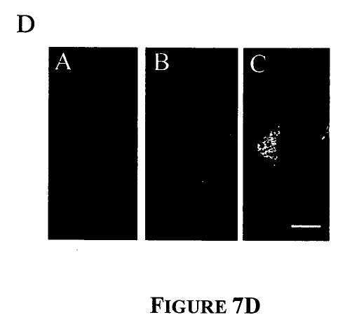

The only difference between these two peptides being that the latter peptide is phosphorylated on serine and this modification prevents it from binding Nck (Zhao et al., 1998). To investigate the effects of peptides we first serum starve the cells for 2-4 h to activate Cdc42Hs and Rac. Unlike control cells which are small, round and morphologically inactive, these partially serum starved N1E-115 cells are generally elongated with short processes and possess active filopodia, lamellipodia and membrane ruffles. Cells injected with αP1 posses neurites that are flattened and morphologically active. However, cells microinjected with the control peptide αP1* are not significantly different from control uninjected cells (FIG. 7A).

αP2

The 13 mer peptide represents the core sequence for Nck interaction. Next we examine the effects of a larger 20 mer peptide, SNNGLDIQDKPPAPPMRNTS (αP2) (SEQ ID NO: 2), from αPAK.

αP2 possesses all the essential amino acids necessary for Nck binding (Zhao et. al., 1998). The phenotypic effect of αP2 is more pronounced than the αP1 (13 mer peptide; FIG. 7B); cells possess neurites that are thinner and longer and a higher percentage of cells possessed neurites. αP2 injected cells closely resemble cells that have been serum starved for 24 h and have taken on a neuronal fate. The effect of αP2 is dosage dependent with 10 μg/ml being optimal for the phenotypic changes (FIG. 7C).

As shown in FIG. 3, neurite outgrowth induced by serum starvation for 24 h or C3 toxin injection (data not shown) is associated with loss of αPAK containing FC's. By double staining αP2 injected cells with vinculin and αPAK antibodies we find that peptide treatment also leads to loss of FC's (FIG. 7D). These results support the idea the αPAK is required for maintenance of Rho-dependent FA's and FC's and plays a role in cell adhesion. The results presented in FIG. 7 establish the use of αP2, a 20 mer peptide derived from the Nck binding site of αPAK, as a inhibitor of PAK function that is potentially αPAK specific.

βP2 and γP2

Next, we test whether peptides, SDSLDNEEKPPAPPLRMNSN (βP2) (SEQ ID NO: 3) and SDNGELEDKPPAPPVRMSST (γP2) (SEQ ID NO: 4), derived from βPAK and γPAK, respectively, also have specific effects on cell morphology.

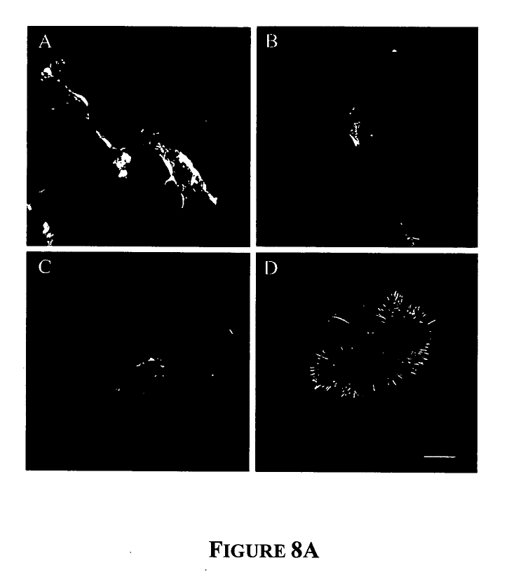

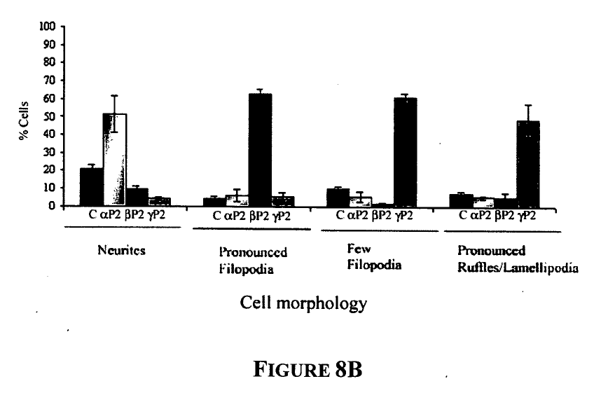

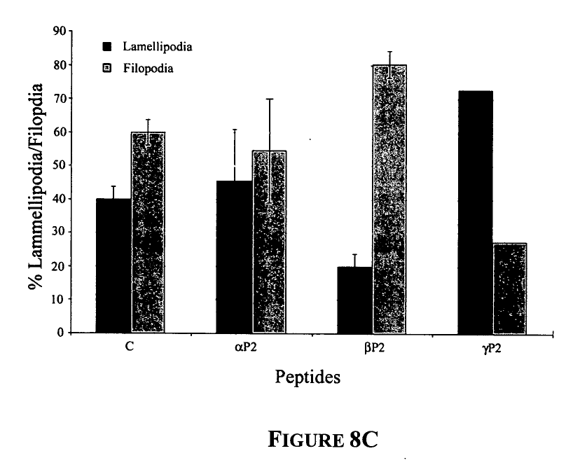

Peptide βP2 injected cells possessed numerous filopodia along a newly formed leading edge with no lamellipodia. Peptide γP2 injected cells possessed a large fan-shaped lamellipodia with no filopodia (FIG. 8A). The striking morphologies (resembling motile keratinocytes) obtained by microinjection of γP2 and βP2 are never found in control cells. The results suggest that peptide βP2 blocks lamellipodia formation while γP2 peptide blocks filopodia formation. In contrast, αP2 peptide seems to synergise with both Cdc42 and Rac to produce neurites. The fact that these three PAK peptides (αP2, βP2 and γP2) give unique morphologies, and control peptides have no effect, suggests that the peptides are acting in an isoform-specific manner.

We carried out a statistical analysis of the phenotypes induced by the peptides by using the bioporter reagent to facilitate entry of peptides into the cell population. The results presented in FIG. 8B support the microinjection experiments. γPAK inhibition leads to loss of filopodia, βPAK inhibition leads to loss of lamellipodia and αPAK inhibition leads to neurite outgrowth.

To confirm that cells micro-injected with γP2 and βP2 peptides have defects in filopodia and lamellipodia formation we undertook a phase contrast time-lapse analysis. Cells are exposed to a gradient of Insulin-like growth factor (IGF) and morphology followed for 60 min scoring for the formation of filopodia and lamellipodia. Cells injected with αP2 respond to IGF morphologically in a manner similar to controls with significant filopodial and lamellipodial activity.

In contrast, cells injected with γP2 and βP2 peptides inhibit the formation of filopodia and lamellipodia, respectively (FIG. 8C). Taken together, the results presented in FIGS. 7 and 8 show that α, β and γPAK isoforms control different aspects of cell morphology and thereby must play distinct roles in cell motility.

### Example 8

### Role of PAK Isoforms in Axonal Guidance

Having established that peptides are specific in their effects on cell morphology and can be used as PAK isoform-specific inhibitors we next examine the effect of these peptides on axonal guidance.

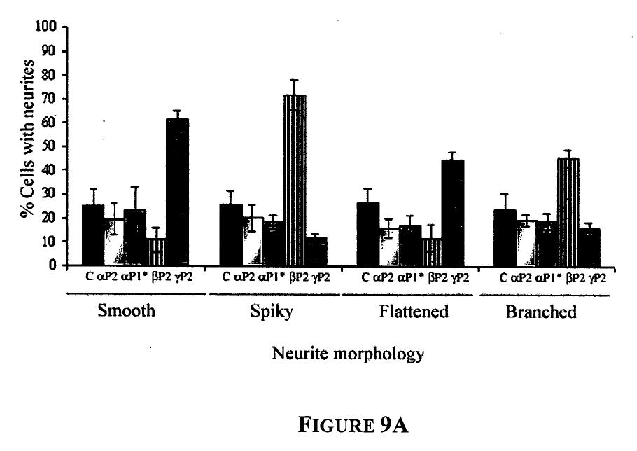

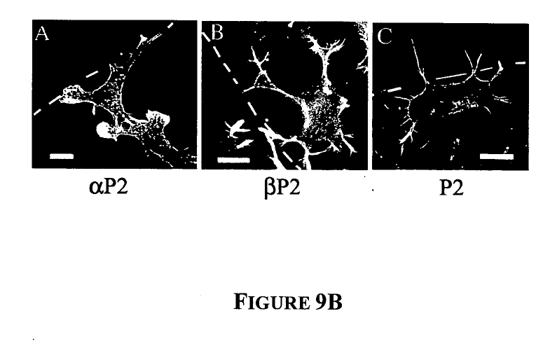

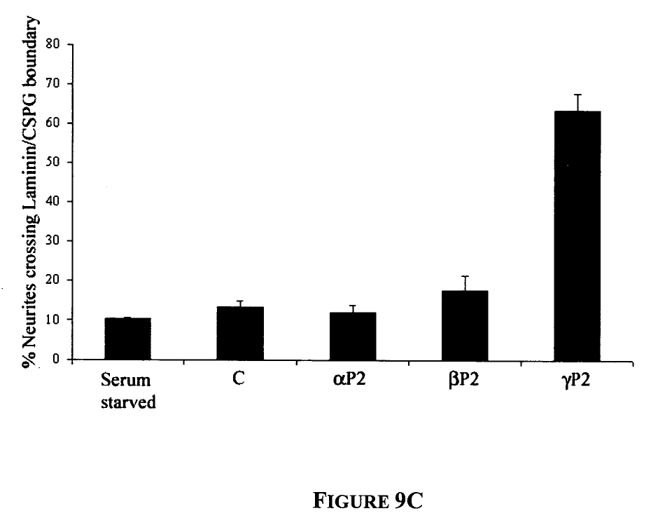

Neurofilament positive neurites are scored for how they respond to Laminin/CSPG boundary. Control neurites did not cross the boundary generally and give a score of approx. 10%. The peptides do not inhibit neurite outgrowth itself over the time course of the assay (data not shown). However, neurites do have distinct morphologies (FIG. 9A). Thus α, β and γ PAK are not essential for neurite outgrowth.

The most striking morphology is obtained with βP2; cells are branched and spiky suggesting that they have lost polarity and possess many filopodia. αP2/αP1* cells have morphologies indistinguishable from control cells. Since serum starvation fully activates Cdc42 and Rac, it is likely that αPAK, under these conditions, does not have a function in determining cell morphology. γP2 cells are smooth and flattened with fan shaped growth cones. The neurite morphologies generated by the PAK peptides are consistent with a role for γPAK in filopodia formation and βPAK in lamellipodia formation as seen in experiments presented in FIG. 8.

Next we examine the role of PAK isoforms in making guidance decisions. Cells incubated with αP2 and βP2 are not affected in their ability to detect the boundary. In contrast, γP2 cells do not to recognize the boundary with 60-70% crossing over and thereby are defective in the decision making process (FIG. 9C).

### Example 9

### Discussion: Relationship between Cdc42Hs/Rac1 and RhoA in Mammalian Cells

In Swiss 3T3 cells bradykinin and Cdc42Hs induce the formation of peripheral F-actin microspikes as well as a loss of stress fibres (Kozma et al., 1995). The morphological effects of bradykinin are inhibited by dominant negative Cdc42Hs and by the RhoA activator LPA (Kozma et al., 1995). In N1E-115 cells, it has been shown that Cdc42Hs induced filopodia formation is inhibited by RhoA and that the RhoA inhibitor C3 toxin induces the formation of filopodia and lamellipodia by activating Cdc42Hs and Rac1.

Furthermore, LPA (RhoA) driven growth cone collapse is inhibited by the presence of the Cdc42Hs/Rac1 activator Acetylcholine (Kozma et al., 1997). In Bac1 cells C3 toxin induces the formation of filopodia and lamellipodia within minutes of microinjection. By 24 h process outgrowth and differentiation is seen. In Bac1 cells LPA induced collapse of cell processes, a phenomenon analogous to neurite collapse in N1E-115 cells, is blocked by the presence of CSF, an activator Cdc42Hs and Rac1.

Taken together, these results show that there is an antagonistic relationship between Rho family GTPase's in terms of their rapid morphological effects. RhoA's role appears to be in cell rounding and adherence while that of Cdc42Hs and Rac1 is to induce cell extension. It is important to consider these antagonistic interactions between Rho family GTPases when examining their function.

In N1E-115 and PC12 cells C3 has been shown to induce cell differentiation (Jalink et al., 1994; Morii and Narumiya, 1995). Notably, in N1E-115 cells C3 toxin induces neurite outgrowth in the presence of serum by activating Cdc42Hs and Rac1 (Kozma et al., 1997). In Bac1 cells serum withdrawal in the presence of CSF at 24 h induces process outgrowth and morphological differentiation. Similarly, in N1E-115 cells serum withdrawal leads to neurite outgrowth and growth inhibition. In N1E-115, PC12 and Bac1 cells the data suggest that RhoA dominance is required for cell growth while Cdc42Hs and Rac1 dominance are required for cell differentiation.

Thus, Rho family proteins appear to control not only rapid remodelling of F-actin cytoskeleton but also the developmental switch between cell growth and cell differentiation.

### Example 10

### Discussion: PAK Isoforms and FCs/FAs

The observation that drosophila PAK localises with phosphotyrosine in multi-protein complexes at the leading edge of cells involved in dorsal closure suggested that it might be a component of FCs (Harden et al., 1996). Mammalian PAKs possess N-terminal sequences that can localise them to FCs and FAs (Manser et al., 1997).

Overexpression of Rac1 and Cdc42Hs in HeLa cells induces recruitment of transfected PAK to peripheral FCs. The Rac exchange factor PIX seems to play an important role in localising PAK to these multimeric protein aggregates (Manser et al., 1998;Oberimeir et al., 1998). Another protein p95PKL/GIT, an ARF-GAP like protein which binds paxillin and PIX, allows the PAK to localize to FC's (Turner et. al., 1999; Manser and Lim, 1999). Recent work suggests that the PAK P4 sequence allows focal complex localization through binding to a PIX-(GIT2/PKL)-paxillin complex independently of Nck (Brown et. al., 2002).

Bac1 cells possess high levels of endogenous PAK making it an ideal system to examine whether endogenous PAKs are components of FCs. We found that α, β and γPAK isoforms colocalise with vinculin, a component of FCs, at distinct sites. γPAK marked preferentially rib-like structures and filopodia, βPAK membrane ruffles/lamellipodia and αPAK localised with vinculin in complexes present in the centre/nuclear region of the cell. Interestingly, in the three different cell types studied αPAK did not colocalise with vinculin at the cell periphery with filopodia/lamellipodia or areas of membrane ruffling.

By comparing αPAK/vinculin co-staining in conditions where RhoA is active (LPA/serum without CSF) with conditions where it is inactive (by microinjection C3 toxin, or serum starvation in the presence of CSF) we are able to show that the αPAK isoform is a component of FCs requiring RhoA activity in Bac1 cells. The αPAK associated FCs contained paxillin, phosphotyrosine and α1/β6 integrins.

In Swiss 3T3 cells αPAK is recruited to RhoA-dependent FAs during assembly (spreading cells) and disassembly (bradykinin-treated cells) suggesting that αPAK is involved in both the formation of FAs and their disassembly. Thus PAK isoforms play a role in the dynamics of the FCs not only involved in cell extension but also cell adhesion (FAs).

### Example 11

### Discussion: PAK Isoforms and F-Actin

Overexpression of αPAK in Swiss 3T3 cells has been found to induce changes in F-actin structures, namely the formation of membrane ruffles, via a kinase and Cdc42Hs/Rac1-independent mechanism (Sells et al. 1997).

As shown in the above Examples, in Bac1 and N1E-115 cells we find that PAK isoforms have distinct localisations. βPAK is found to colocalise most strongly with F-actin in lammelipodia/membrane ruffles while γPAK colocalised most strongly with filopodia.

αPAK does not localise to filopodia or lamellipodia/membrane ruffles and is never found on the cell periphery with these F-actin based structures. Rather αPAK colocalised with small F-actin clusters in the centre of the cell and with stress fibres. The function of these clusters is unclear but they may act as a reservoir for F-actin filaments and other cytoskeletal components or be involved in cell adherence.

Dharmawardhane et al., (1997) have suggested that αPAK in Swiss 3T3 cells is present at the leading edge associated with membrane ruffles and lamellipodia. In contrast, our studies of the three different cell lines, including Swiss 3T3, under various conditions, fail to show αPAK colocalising with F-actin in filopodia or lamellipodia/membrane ruffles at the leading edge.

We find αPAK to localize with stress fibres during formation and disassembly but not with stable stress fibres. PAK can phosphorylate, myosin heavy chain (Brzeska et al., 1997), myosin light chain kinase (Sanders et. al., 1999) and LIM kinase, a cofilin/ADF kinase (Daniels and Bokoch, 1999). Our results suggest that PAK isoforms have specific and distinct roles in remodeling the major F-actin structures present in cells including stress fibres.

### Example 12

### Discussion: PAK Isoforms Play Distinct Roles in Cell Motility

The localization results suggest important and distinct roles for PAK isoforms in controlling cell morphology dynamics. Moreover, the localization results show that PAK isoforms represent the first unique components of the different types FC's present within cells.

To define the role of PAK isoforms more precisely we chose to try and inhibit endogenous protein function by using peptide inhibitors of protein-protein interaction. The peptides are derived for the N-terminal region (aa's 1-22, P1) of PAK isoforms and this region is involved in binding one of the SH3 domains of the adaptor protein Nck.

The peptides should alter the ability of PAK to interact with Nck and since this interaction has been shown to be important for PAK function/localisation (Hing et. al., 1999, Ruan et., al. 1999) would be predicted to inhibit PAK function in vivo. However, the mechanism by which the peptides give rise to specific inhibition of PAK isoforms function is unclear.

To date, two isoforms of Nck, α and β, have been identified with each isoform having a different cellular localization/function (Chen et. al., 2000). Clearly this in itself is insufficient to provide three different phenotypes observed with the PAK peptides. One possibility is that additional isoform-specific targets or Nck related molecules play a role in interaction with N-terminal 1-25 aa's of PAK isoforms. Further work is necessary to resolve how the peptides act as isoform-specific inhibitors.

In the Examples, the peptides are introduced into cells by either microinjection or using the bioporter system. The latter technique is particularly powerful as it allows whole populations of cells to be studied rapidly with minimal perturbance. The phenotypes obtained by peptides α, β, and γ (P2) are distinct from each other and not found in the general cell population.

Taken together with the localization data the results suggest that γPAK is important for filopodia formation, βPAK for lamellipodia/membrane ruffles formation and αPAK for cell adhesion and cell rounding. An unexpected observation made here is that αPAK, a Cdc42 and Rac1 effector, is intimately involved in regulating the turnover of RhoA-dependent FA's, FC's and stress fibres and thereby levels of cell adhesion.

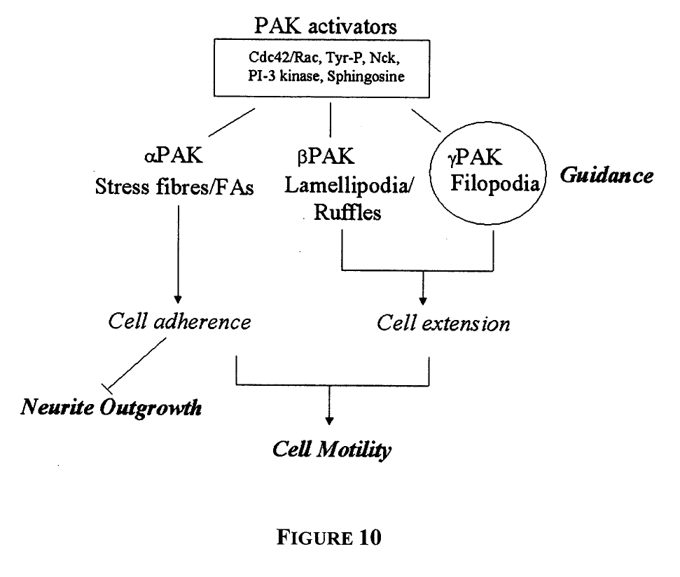

Together with the roles suggested here for γ and βPAK in cell extension a model for how Cdc42Hs and Rac1 might control cell motility through co-ordination of these three PAK isoform is presented (FIG. 10). Regulation of cell motility by Cdc42 and Rac is likely to involve additional layers of complexity, including control of effector complexes associated with proteins such as IRS-58, N-WASP and MRCK.

### Example 13

### Discussion: PAK Isoforms Play Distinct Roles in Axonal Guidance

The Examples also show experiments which examine the effect of these isoform-specific peptides inhibitors on axonal guidance using a matrix boundary assay.

The peptide data suggest that PAK isoforms, at least individually, are not essential for neurite outgrowth. However, βP2 and γP2, but not αP2 did affect neurite morphology. In the presence of βP2 neurites are branched and spiky with lamellipodia and ruffles inhibited while in the presence of γP2 neurites had flattened growth cones and very few filopodia. The data suggest that γPAK but not α or β, is essential for making the guidance decisions.

This result is consistent with the view that filopodia play an important role in guidance decisions (Bentley and Torian-Raymond, 1986; Chien et. al., 1993). In preliminary experiments we have also examined the effect of the PAK peptides on axonal guidance towards IGF in N1E-115 cells. The data are consistent with the laminin/CSPG matrix boundary assay; αP2 and βP2 do not affect guidance while γP2 does. Interestingly, the IGF guidance experiment revealed that βP2 does slow down the decision making process of moving towards IGF suggesting a supportive but not essential role for this PAK isoform in axonal guidance (K. Marler and S. Ahmed, unpublished data).

Inhibitory cues, such as CSPG, Oligodendrocyte myelin glycoprotein (OMgp), and Myelin associated Glycoprotein (MAG), in the central nervous system are thought to prevent axonal regrowth (Kruger and Morrison, 2002). In this study we have identified γP2 as a cell signaling inhibitor of axonal guidance which may have utility in stimulating nerve regeneration. The isolation of peptides that block protein-protein interactions and can act as specific inhibitors of complex cell signaling pathways may open up new ways to aid nervous system repair. In future experiments, the isoform-specific peptide inhibitors of PAK function will be invaluable for investigating the roles of these proteins in cell function and disease states.

In conclusion, this is the first study that has compared the endogenous cellular localization of PAK isoforms. The data clearly show that α, β and γPAK isoforms are present at locations that may allow them to play different roles in cell motility. The use of isoform-specific peptide inhibitors confirms that α, β and γPAK isoforms do indeed play distinct roles in cell motility and further that γPAK is essential for axonal guidance.

The invention is further described by the following numbered paragraphs:

1. An isoform specific antagonist of PAK kinase.

2. An isoform specific antagonist of PAK kinase according to paragraph 1, which comprises a molecule capable of modulating an interaction between an SH3 containing polypeptide and a PAK isoform.

3. An isoform specific antagonist of PAK kinase according to paragraph 1 or 2, which comprises a molecule capable of preventing or interfering with the binding of Nck, preferably Nckα or Nckβ, to a PAK isoform.

4. An isoform specific antagonist of PAK kinase according to paragraph 1, 2 or 3, which comprises a peptide, preferably comprising a sequence from a PAK kinase isoform, preferably a sequence having an accession number U23443, O88643 or Q 13153 (αPAK), U33314, CAD42791 or O75914 (βPAK) or Q64303, AN65624, or NP_{—}002568 (γPAK).

5. An isoform specific antagonist of PAK kinase according to paragraph 4, in which the peptide comprises an Nck binding domain.

6. An isoform specific antagonist of PAK kinase according to paragraph 4 or 5, in which the peptide comprises an isoform specific domain.

7. An isoform specific antagonist of PAK kinase according to paragraph 4, 5 or 6, in which the peptide comprises a N-terminal portion of a PAK kinase isoform, preferably residues 2 to 21 of a PAK kinase isoform.

8. An isoform specific antagonist of PAK kinase according to any of paragraphs 4 to 7, in which the peptide is selected from the group consisting of: EDKPPAPPMRNTSMI (αP1) (SEQ ID NO: 1), SNNGLDIQDKPPAPPMRNTS (αP2) (SEQ ID NO: 2), SDSLDNEEKPPAPPLRMNSN (βP2) (SEQ ID NO: 3) and SDNGELEDKPPAPPVRMSST (γP2) (SEQ ID NO: 4).

9. An isoform specific antagonist of PAK kinase according to any of paragraphs 4 to 8, in which the peptide comprises a sequence from Nck, preferably a PAK binding portion of Nck.

10. An isoform specific antagonist of PAK kinase according to paragraph 1, 2 or 3, which comprises an antibody.

11. An isoform specific antagonist of PAK kinase according to paragraph 10, in which the antibody is selected from the group consisting of: antibody sc-882, antibody sc-1871, antibody sc-1872 and antibody sc-7117 (Santa Cruz Biotechnology, Inc).

12. An isoform specific antagonist of PAK kinase according to any preceding claim, which is capable of at least one activity selected from the group consisting of: inducing loss of a cell adhesion complex, activating cell spreading, activating cell extension, activating neurite outgrowth, prevention of filopodia formation, and prevention of lamellipodia formation.

13. An isoform specific antagonist of PAK kinase according to any preceding claim, which comprises a αPAK isoform specific antagonist.

14. An isoform specific antagonist of PAK kinase according to paragraph 13, which is capable of stimulating neurite outgrowth (neuritogenesis).

15. An isoform specific antagonist of PAK kinase according to paragraph 13 or 14, which is capable of stimulating nerve regeneration.

16. An isoform specific antagonist of PAK kinase according to any preceding claim, which comprises a γPAK isoform specific antagonist.

17. An isoform specific antagonist of PAK kinase according to paragraph 16, which is capable of inhibiting axonal guidance.

18. An isoform specific antagonist of PAK kinase according to paragraph 16 or 17, which is capable of allowing neurite outgrowth across an attractive/repulsive boundary, preferably a laminin/CSPG boundary.

19. A peptide having the sequence EDKPPAPPMRNTSMI (αP1) (SEQ ID NO: 1), SNNGLDIQDKPPAPPMRNTS (αP2) (SEQ ID NO: 2), SDSLDNEEKPPAPPLRMNSN (βP2) (SEQ ID NO: 3) and SDNGELEDKPPAPPVRMSST (γP2) (SEQ ID NO: 4).

20. A nucleic acid comprising a sequence capable of encoding an isoform specific antagonist of PAK kinase according to any of paragraphs 1 to 18 or a peptide according to paragraph 19.

21. An expression vector comprising a nucleic acid sequence according to paragraph 20.

22. A method of inhibiting an activity of an isoform of PAK kinase, the method comprising contacting an isoform of PAK kinase with an isoform specific antagonist of PAK kinase.

23. A method according to paragraph 22, in which the isoform of PAK kinase is contacted with an isoform specific antagonist of PAK kinase according to any of paragraphs 1 to 18, a peptide according to paragraph 19, a nucleic acid according to paragraph 20 or an expression vector according to paragraph 21.

24. A method of inhibiting an activity of an αPAK kinase isoform, the method comprising contacting an αPAK kinase with an αPAK isoform specific antagonist.

25. A method of stimulating neurite outgrowth, the method comprising contacting a cell with an αPAK isoform specific antagonist.

26. A method of stimulating nerve regeneration, the method comprising contacting a cell with an αPAK isoform specific antagonist.

27. A method according to paragraph 24, 25 or 26, in which the αPAK isoform specific antagonist comprises a peptide EDKPPAPPMRNTSMI (αP1) (SEQ ID NO: 1), SNNGLDIQDKPPAPPMRNTS (αP2) (SEQ ID NO: 2) or an antibody sc-882 (Santa Cruz Biotechnology, Inc).

28. A method of inhibiting an activity of an γPAK kinase isoform, the method comprising contacting an γPAK kinase with an γPAK isoform specific antagonist.

29. A method of inhibiting axonal guidance, the method comprising contacting a cell with a γPAK isoform specific antagonist.

30. A method of enabling neurite outgrowth across an attractive/repulsive boundary, preferably a laminin/CSPG boundary, the method comprising contacting a cell with a γPAK isoform specific antagonist.

31. A method according to paragraph 28, 29 or 30, in which the γPAK isoform specific antagonist comprises a peptide SDNGELEDKPPAPPVRMSST (γP2) (SEQ ID NO: 4), an antibody sc-1872 or antibody sc-7117 (Santa Cruz Biotechnology, Inc).

32. Use of an isoform specific antagonist of PAK kinase for specifically inhibiting an activity of an isoform of PAK kinase.

33. Use according to paragraph 32, in which the isoform specific antagonist of PAK kinase comprises an isoform specific antagonist of PAK kinase according to any of paragraphs 1 to 18, a peptide according to paragraph 19, a nucleic acid according to paragraph 20 or an expression vector according to paragraph 21.

34. Use of an αPAK isoform specific antagonist of PAK kinase for specifically inhibiting an activity of αPAK kinase.

35. Use of an αPAK isoform specific antagonist of PAK kinase for stimulating neurite outgrowth.

36. Use of an αPAK isoform specific antagonist of PAK kinase for stimulating nerve regeneration

37. Use according to paragraph 34, 35 or 36, in which the αPAK isoform specific antagonist comprises a peptide EDKPPAPPMRNTSMI (αP1) (SEQ ID NO: 1), SNNGLDIQDKPPAPPMRNTS (αP2) (SEQ ID NO: 2) or an antibody sc-882 (Santa Cruz Biotechnology, Inc).

38. Use of a γPAK isoform specific antagonist of PAK kinase for specifically inhibiting an activity of γPAK kinase.

39. Use of a γPAK isoform specific antagonist of PAK kinase for inhibiting axonal guidance.

40. Use of a γPAK isoform specific antagonist of PAK kinase for enabling neurite outgrowth across an attractive/repulsive boundary, preferably a laminin/CSPG boundary.

41. Use according to paragraph 38, 39 or 40, in which the γPAK isoform specific antagonist comprises a peptide SDNGELEDKPPAPPVRMSST (γP2) (SEQ ID NO: 4), an antibody sc-1872 or antibody sc-7117 (Santa Cruz Biotechnology, Inc).

42. A method of identifying an isoform specific antagonist of PAK kinase, the method comprising: (a) providing a candidate molecule, (b) contacting the candidate molecule with a peptide comprising an Nck binding portion of a PAK isoform, or a fragment, homologue, variant or derivative thereof, and (c) detecting binding of the candidate molecule to the peptide, fragment, homologue, variant or derivative thereof.

43. A method of identifying an isoform specific antagonist of PAK kinase, the method comprising: (a) providing a candidate molecule, (b) contacting a peptide comprising an PAK binding portion of Nck, or a fragment, homologue, variant or derivative thereof, with a candidate molecule, and (c) detecting the binding of the molecule to the PAK binding peptide, fragment, homologue, variant or derivative thereof.

44. A method of identifying an isoform specific antagonist of PAK kinase, the method comprising: (a) providing a candidate molecule, (b) contacting the candidate molecule to a peptide comprising an Nck binding portion of a PAK isoform, or a fragment, homologue, variant or derivative thereof, and (c) detecting modulation of activity of the peptide, fragment, homologue, variant or derivative thereof.

45. A method according to paragraph 42, 43 or 44, in which the PAK isoform is αPAK, γPAK, or both.

46. A method according to any of paragraphs 42 to 45, in which the activity comprises a γPAK isoform specific activity, preferably selected from the group consisting of: maintenance of axonal guidance, and inhibition of neurite outgrowth across an attractive/repulsive boundary, preferably a laminin/CSPG boundary.

47. A method according to any of paragraphs 42 to 46, in which the method further comprises providing Nck, or a fragment, homologue, variant or derivative thereof, and contacting the peptide comprising an Nck binding portion of PAK, or a fragment, homologue, variant or derivative thereof with Nck, or a fragment, homologue, variant or derivative thereof in the presence of the candidate molecule, and selecting candidate molecules which modulate binding between the peptide and Nck.

48. A method according to any of paragraphs 42 to 47, in which the method further comprises isolating or synthesising a selected or identified molecule.

49. A isoform specific antagonist of PAK kinase identified by a method according to any of paragraphs 42 to 48.

50. A pharmaceutical composition comprising an isoform specific antagonist of PAK kinase according to any of paragraphs 1 to 18 and 49, a peptide according to paragraph 19, or a nucleic acid according to paragraph 20 or 21, together with a pharmaceutically acceptable excipient or carrier.

51. Use of an isoform specific antagonist of PAK kinase in a method of treatment, prophylaxis or diagnosis of a disease in an individual.

52. A method of treatment of an individual suffering or likely to suffer from a disease, the method comprising administering a therapeutically or prophylactically effective amount of an isoform specific antagonist of PAK kinase to the individual.

53. An isoform specific antagonist of PAK kinase for use in a method of treatment, prophylaxis or diagnosis of a disease in an individual.

54. Use of an isoform specific antagonist of PAK kinase in the preparation of a pharmaceutical composition for the treatment of a disease.

55. A use according to paragraph 54 or 55, a method according to paragraph 42, or an isoform specific antagonist of PAK kinase for a use as specified therein according to paragraph 53 in which the disease is characterised by a defect in nerve regeneration or repair.

56. A use, method or isoform specific antagonist of PAK kinase according to any of paragraphs 51 to 55, in which the disease is selected from the group consisting of: neurodegenerative disorder, an amyloidosis, a tauopathy, Alzheimer's disease, Parkinson's disease (PD), dementia with Lewy Bodies (DLB), frontotemporal dementia (FTD), pallido-ponto-nigral degeneration (PPND), familial progressive subcortical gliosis, and familial multisystem tauopathy (FMT), familial progressive dementia with psychosis, pallido-nigral degeneration and bipolar disorder (manic depressiveness).

57. A use, method or isoform specific antagonist of PAK kinase according to any of paragraphs 51 to 55, in which the isoform specific antagonist of PAK kinase is an αPAK isoform specific antagonist, or a γPAK isoform specific antagonist, or both.

58. A method of treatment of an individual suffering or likely to suffer from a disease preferably characterised by a defect in nerve regeneration, the method comprising modulating an activity of αPAK kinase, or modulating an activity of γPAK kinase, or both.

59. A method of treatment according to paragraph 58, in which the method comprises inhibiting an interaction, preferably a binding interaction, between αPAK kinase and Nck, or between γPAK kinase and Nck or both.

60. A method according to paragraph 59, in which the disease is selected from the group consisting of diseases set out in paragraph 56.

61. A method of modulating cellular guidance, preferably axonal guidance, the method comprising exposing a cell, preferably a nerve cell, to an isoform specific antagonist of PAK kinase.

62. Use of an αPAK isoform specific antagonist in a method of stimulating neural outgrowth.

63. Use of a γPAK isoform specific antagonist in a method of modulating axonal guidance.

64. Use of an isoform specific anti-PAK antibody as an isoform specific antagonist of PAK kinase.

65. A combination of a PAK isoform specific antagonist together with a nerve cell.

66. A combination according to paragraph 65, in which the PAK isoform specific antagonist comprises an αPAK isoform specific antagonist, or a γPAK isoform specific antagonist, or both.

67. A kit comprising an isoform specific antagonist of PAK kinase, together with packaging components and instructions for use in nerve regeneration.

68. An isoform specific antagonist of PAK kinase substantially as hereinbefore described with reference to and as shown in the accompanying drawings.

69. A nucleic acid encoding an isoform specific antagonist of PAK kinase substantially as hereinbefore described with reference to and as shown in the accompanying drawings.

70. An expression vector substantially as hereinbefore described with reference to and as shown in the accompanying drawings.

71. A method of identifying an isoform specific antagonist of PAK kinase substantially as hereinbefore described with reference to and as shown in the accompanying drawings.

### REFERENCES

Abo, A, Qu, J., Cammarano, M. S., Dan, C., Fritsch, A., Baud, V., Belisle, B., and Minden, A. (1998) PAK4, a novel effector for Cdc42Hs, is implicated in the reorganisation of the actin cytoskeleton and in the formation of filopodia. EMBO J., 17, 6527-6540.

Ahmed, S., Kozma, R., Hall, C., and Lim, L. (1995) GAP activity of n-chimaerin and effect of lipids. Methods in Enzymol., 256, 114-125.

Ahmed, S., Lee, J., Wen, L-P., Zhao, Z-S., Ho, J., Best, A., Monfries, C., Kozma, R., and Lim, L. (1994) Breakpoint cluster region gene product-related domain of n-chimaerin. Discrimination between Rac-binding and the GTPase-activating residues by mutational analysis. J. Biol. Chem., 269, 17642-17648.

Allen, W. E., Jones, G. E., Pollard, J. W. and Ridley, A. J. (1997) Rho, Rac1 and Cdc42Hs regulate actin organisation and cell adhesion in macrophages. J. Cell. Science, 110, 707-720.

Bentley, D., and Toroian-Raymond, A., (1986) Disoriented pathfinding by pioneer growth cones deprived of filopodia by cytochalasin treatment. Nature 323, 712-715.

Brown, M. C., west, K. A., and Turner, C. E. (2002) Paxillin-dependent Paxillin Kinase Linker and p21-Activated Kinase Localisation to Focal Adhesions Involves a Multistep Activation Pathway. Mol. Biol. Cell 13, 1550-1565.

Brzeska, H., Kaus, U. G., Wang, Z-Y., Bokoch, G. M., and Korn E. D. (1997) p21-activated kinase has substrate specificity similar to Acanthamoeba myosin 1. Proc. Natln. Acad. Sci. USA, 94, 1092-1095.

Bokoch, G. M., Wang, Y., Bohl, B. P., Sells, M. A., Quilliam, L. A. and Knaus, U. G. (1996) Interaction of Nck adaptor protein with p21-activated kinase (PAK1). J. Biol. Chem., 271, 25746-25749.

Chen, M., She, H., Kim, A., Woodley, D. T., and Wei, L. (2000) Nckβ Adaptor Regulates Actin Polymerisation in NiH sts Fibroblasts in Response to Platelet-Derived Growth Factor bb. Mol. Cell. Biol. 20, 7867-7880.

Chien, C. B., Rosnethal, D. E., Harris, W. A., and Holt, C. E. (1993) Navigational errors made by growth cones without filopodia in the embryonic Xenopus brain. Neuron 11, 237-251.

Daniels, R. H. and Bokoch, G. M. (1999) p21-activated protein kinase: a crucial component of morphological signaling? Trends Biochem. Sci., 24, 350-354.

Daniels, R. H., Hall, P. S. and Bokoch, G. M. (1998) Membrane targeting of p21-activated kinase 1 (PAK1) induces neurite outgrowth from PC12 cells. EMBO J., 17, 754-764.

Dharmawardhane, S., Sanders, L. C., Martin, S. S., Daniels, R. H., and Bokoch, G. (1997) Location of p21-Activated Kinase1 (PAK1) to Pinocytic Vesicles and cortical Actin Structures in Stimulated Cells. J. Cell. Biol., 138, 1265-1278.

Galisteo, M. L., Chernoff, J., Su, Y.-C., Skolnik, E. Y. and Schlessinger, J. (1996) The adaptor protein Nck links tyrosine kinases with the serine-threonine kinase Pak1. J. Biol. Chem., 271, 20997-21000.

Govind, S. Kozma, R., Monfires, C., Lim, L., and Ahmed, S. (2001) Cdc42Hs facilitates cytoskeletal rearrangements and neurite outgrowth by localizing 58 kD Insulin Receptor Substrate to Filamentous actin. J. Cell. Biol. 152, 579-594.

Hall, A. (1994) Small GTP-binding protein and regulation of the actin cytoskeleton. Annu. Rev. Cell Biol., 10, 31-54.

Hall, A. (1998) Rho GTPases and the actin Cytoskeleton. Science, 279, 509-514.

Harden, N., Lee, J., Loh, H-Y., Ong, Y-M., Tan, I., Leung, T., Manser, E. and Lim, L. (1996) Drosophila homolog of the Rac- and Cdc42Hs-activated serine/threonine kinase PAK is a potential focal adhesion and focal complex protein that colocalises with dynamic actin structure. Mol. Cell. Biol., 16, 1896-1908.

Hing, H., Xiao, J., Harden N., Lim, L., and Zipursky, S. L., (1999). PAK Functions Downstream of Dock to Regulate Photoreceptor Axon Guidance in Drosophila. Cell 97, 853-863.

Hirose, M., Ishizaki, T., Watanabe, N., Uehata, M., Kranenburg, O., Moolenaar, W. H., Matsumura, F., Maekawa, M., Bito, H., and Narumiya, S. (1998) Molecular dissection of the Rho-associated protein kinase p160ROCK-regulated neurite remodeling in Neuroblastoma N1E-115 cells. J. Cell. Biol., 141, 1625-1636.

Jalink, K., Van Corven, E. J., Hengeveld, T., Morii, N., Narumiya, S., and Moolenaar, W. H. (1994) Inhibition of LPA-induced and Thrombin-induced neurite retraction and neuronal cell rounding by ADP-ribosylation of the small GTP binding protein Rho. J. Cell. Biol., 126, 801-810.

Kiosses, W. B., Daniels, R. H., Otey, C., Bokoch, G., and Schwartz, M. A. (1999). A role for p21-activated kinase in endothelial cell migration. J. Cell. Biol. 147, 831-843.

Knaus, U. G., Morris, S., Dong, H-J., Chemoff, J. and Bokoch, G. M. (1995) Regulation of Human Leukocyte p21-Activated Kinases Through G Protein-Coupled Receptors. Science, 269, 221-223.

Kozma, R. Ahmed, S. Best, A. and Lim, L. (1995) The Ras related protein Cdc42Hs and bradykinin promote the formation of peripheral actin microspikes and filopodia in Swiss 3T3 fibroblasts. Mol. Cell. Biol., 15, 1942-1952.

Kozma, R., Ahmed, S. Best, A. and Lim, L. (1996) The GTPase activating protein n-chimaerin cooperates with Rac1 and Cdc42Hs to induce the formation of Lamellipodia and Filopodia. Mol. Cell. Biol., 16, 5069-5080.

Kozma, R. Sarner, S., Ahmed, S. and Lim, L. (1997) Rho family GTPases and neuronal growth cone remodeling:relationship between increased complexity induced by Cdc42Hs, Rac1 and Acetylcholine and collapse induced by RhoA and Lysophosphatidic acid. Mol. Cell. Biol., 17, 12011-1211.

Kruger, G. M. and Morrison, S. J. (2002) Brain Repair by Endogenous Progenitors. Cell 110, 399-402.

Lamarche, N., Tapon, N., Stowers, L., Burbelo, P. D., Aspenstrom, P., Bridges, T., Chant, J. and Hall, A. (1996) Rac and Cdc42 induce actin polymerisation and G1 cell cycle progression independently of p65PAK and the JNK/SAPK MAP kinase cascade. Cell, 87, 519-529.

Leung, T., Chen, X-Q., Tan, I., Manser, E., and Lim, L. (1998) The myotonic dystrophy kinase-related Cdc42-binding kinase (MRCK) as a Cdc42 effector in promoting cytoskeletal reorganisation. Mol. Cell. Biol., 18, 130-140.

Lim, L., Manser, E., Leubg, T. and Hall, C. (1996) Regulation of phosphorylation pathways by p21GTPases. The Ras related Rho subfamily and its role in phosphorylation signalling pathways. Eur. J. Biochem, 242, 171-185.

Llic, D., Damsky, C. H., and Yamamoto, T. (1997) Focal adhesion kinase: at the crossroads of signal transduction. J. Cell Science, 110, 401-407.

Lu, W., Katz, S., Gupta, R., Mayer, B. J. (1997) Activation of Pak by membrane localization mediated by an SH3 domain from the adaptor protein Nck. Curr. Biol. 7, 85-94.

Manser, E and Lim, L (1999) Role of PAK family kinases. Prog. Mol. Cell Biol., 22, 114-133.

Manser, E. Leung, T., Salihuddin, H., Zhao, Z. S., and Lim, L. (1994) A brain serine/threonine protein kinase activated by Cdc42 and Rac1. Nature, 367, 40-46.

Manser, E., Chong, C., Zhao, Z-S., Leung, T., Michael, G., Hall, C. and Lim, L. (1995) Molecular Cloning of a New Member of the p21-Cdc42/Rac1-activated Kinase (PAK) family. J. Biol. Chem, 270, 25070-25078.

Manser, E., Huang, H-Y., Loo, T-H., Chen, X-Q., Dong, J-M., Leung, T. and Lim, L. (1997) Expression of constitutively active alpha PAK reveals effects of the kinase on actin and focal complexes. Mol. Cell. Biol., 17, 1129-1143.

Manser, E., Loo, T-H., Koh, C-G., Zhao, Z-S., Chen, X-Q., Tan, L., Tan, I., Leung, T., and Lim, L. (1998) PAK kinases are directly coupled to the PIX family of nucleotide exchange factors. Mol. Cell, 1, 183-192.

Martin, G. A., Bollag, G., McCormick, F., and Abo, A. (1995) A novel serine kinase activated by rac1/CDC42Hs-dependent autophosphorylation is related to PAK65 and STE20. EMBO J., 14, 1970-1978.

Morii, N. and Nerumiya, S. (1995) Preparation of native and recombinant Clostridium botulinum C3 ADP-Robosyltransferase and identification of Rho proteins by ADP-Ribosylation. Methods Enzymol., 256, 196-206.

Nobes, C. D. and Hall, A. (1995) Rho, Rac, and Cdc42 GTPases regulate the assembly of multimolecular focal complexes associated with actin stress fibres, lamellipodia and filopodia. Cell, 81, 53-62.

Obermeier, A., Ahmed, S., Manser, E., Yen, S-C., Hall, C., and Lim, L. (1998) PAK promotes morphological changes by acting upstream of Rac. EMBO J., 17, 101-112.

Prigmore, E., Ahmed, S., Best, A., Kozma, R., Manser, E., Segal, A. W. and Lim, L. (1995) A 68 kDa Kinase and NADPH Oxidase Component p67phox Are Targets for Cdc42Hs and Rac1 in Neutrophils. J. Biol. Chem., 270, 10717-10722.

Ridley, A. J. and Hall, A. (1992) The small GTP-binding protein rho regulates the assembly of focal adhesions and actin stress fibers in response to growth factors. Cell, 70, 389-399.

Ridley, A. J., Paterson, H. F., Johnston, C. L., Diekmann, D., and Hall, A. (1992) The small GTP-binding protein rac regulates growth factor induced membrane ruffling. Cell, 70, 401-410.

Ruan, W., Pang, P., Rao, Y. (1999) The Sh2/Sh3 Adaptor Protein Dock Interacts with the Ste20-like Kinase Misshapen in Controlling Growth Cone Motility. Neuron 24, 595-605.

Sarner, S., Kozma, R., Ahmed, S., and Lim, L. (2000) PI-3 kinase, Cdc42Hs and Rac1 act downstream of Ras in Integrin-dependent neurite outgrowth in N1E-115 neuroblastoma cells Mol. Cell. Biol., 20, 158-172.

Sanders, L. C., Matsumara, F., Bokoch, G. M., and de Lanerolle, P. (1999) Inhibition of myosin light chain kinase by p21-activated kinase. Science, 283, 2083-2085.

Sells, M. A., Knaus, U. G., Amborse, D., Bagrodia, S., Bokoch, G. M., and Chernoff, J. (1997) Human p21 activated kinases regulate actin reorganisation in mammalian cells. Curr. Biol., 7, 202-210.

Sells, M-A. and Chemoff, J. (1997) Emerging from the PAK: the p21-activated protein kinase family. Trends Cell Biol., 7, 162-167.

Symons, M. (1996) Rho family GTPases: the cytoskeleton and beyond. Trends Biochem. Sci., 4, 178-181.

Teo, M., Manser, E. and Lim, L. (1995) Identification and Molecular Cloning of a p21-Cdc42/Rac1-activated Serine/Threonine Kinase That is Rapidly Activated by Thrombin in Platelets. J. Biol. Chem., 270, 26690-26697.

Turner, C. E., Brown, M. C., Perrotta, J. A., Reidy, M. C., Nikolopoulos, S. N., Mcdonald, A. R., Bagrodia, S., Thomas, S., and Leventhal, P. (1999) Paxillin LD4 motif binds PAK and PIX through a novel 95 kDa Ankyrin repeat, ARF-GAP protein: A role in cytoskeletal remodeling. J. Cell. Biol., 145, 851-863.

VanAeist, L. and D'Souza-Schorey, C. (1997) Rho GTPases signaling networks. Genes Dev., 11, 2295-2322.

Zhao, Z S, Manser, E, Chen, X-Q., Chong, C., Leung, T. and Lim, L.(1998) A conserved negative regulatory region in alpha PAK: Inhibition of PAK kinases reveals their morphological roles downstream of Cdc42 and Rac1. Mol. Cell Biol., 18, 2153-2163.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

We claim:

1. A peptide isoform specific antagonist of PAK kinase, wherein the peptide comprises: (a) an Nck binding domain of PAK kinase and (b) an isoform specific domain of PAK kinase, wherein each of the Nck binding domain and the isoform specific domain comprises a sequence from residues 1 to 24 of a PAK kinase isoform.

2. The peptide according to claim 1, wherein the PAK kinase isoform has a sequence selected from the group consisting of SEQ ID NOs: 6-16.

3. The peptide according to claim 1, wherein the peptide comprises a sequence selected from the group consisting of residues 1 to 21, residues 2 to 21, residues 1 to 20 and residues 2 to 20 of the PAK kinase isoform.

4. The peptide according to claim 1, wherein the Nck binding domain comprises SEQ ID NO: 5.

5. The peptide according to claim 1, wherein the Nck binding domain is selected from the group consisting of: PAPPMRNTS (SEQ ID NO: 34), PAPPVRMSS (SEQ ID NO: 35), PAPPLRMNS (SEQ ID NO: 36), PAPPMRNTS (SEQ ID NO: 37), PAPPVRMSS (SEQ ID NO: 38), PAPPLRMNS (SEQ ID NO: 39), PAPPMRNTS (SEQ ID NO: 40), PAPPVRMSS (SEQ ID NO: 41) and PAPPLRMNS (SEQ ID NO: 42).

6. The peptide according to claim 1, wherein the PAK kinase isoform is an αPAK, and the isoform specific domain sequence is selected from the group consisting of:

| | |
|---|---|
| MSNNGLDVQDKP, | (SEQ ID NO: 43) |
| MSNNGLDIQDKP, | (SEQ ID NO: 44) |
| MSNNGVDIQDKP, | (SEQ ID NO: 45) |
| SNNGLDVQDKP, | (SEQ ID NO: 46) |
| SNNGLDIQDKP | (SEQ ID NO: 47) |
| and | |
| SNNGVDIQDKP. | (SEQ ID NO: 55) |

7. The peptide according to claim 6, wherein the peptide comprises EDKPPAPPMRNTSMI (αP1; SEQ ID NO: 63) or SNNGLDIQDKPPAPPMRNTS (αP2; SEQ ID NO: 27).

8. The peptide according to claim 1, wherein the PAK kinase isoform is a βPAK, and the isoform specific domain sequence is selected from the group consisting of: MSDSLDNEEKP (SEQ ID NO: 48), MSDSLDNEEKPNN (SEQ ID NO: 49), MSDGLDNEEKPNN (SEQ ID NO: 50), SDSLDNEEKP (SEQ ID NO: 51), SDSLDNEEKPNN (SEQ ID NO: 52) and SDGLDNEEKPNN (SEQ ID NO: 53).

9. The peptide according to claim 8, wherein the peptide comprises the sequence SDSLDNEEKPPAPPLRMNSN (βP2; SEQ ID NO: 3).

10. The peptide according to claim 1, wherein the PAK kinase isoform comprises γPAK, and the isoform specific domain sequence is selected from the group consisting of:

| | |
|---|---|
| MSDNGELEDKPTI, | (SEQ ID NO: 54) |
| MSDNGELEDKP, | (SEQ ID NO: 56) |
| SDNGELEDKPTI | (SEQ ID NO: 57) |
| and | |
| SDNGELEDKP. | (SEQ ID NO: 58) |

11. The peptide according to claim 10, wherein the peptide comprises the sequence SDNGELEDKPPAPPVRMSST (γP2; SEQ ID NO: 4).

12. A peptide comprising EDKPPAPPMRNTSMI (αP1); SEQ ID NO: 63), SNNGLDIQDKPPAPPMRNTS (αP2; SEQ ID NO: 27), SDSLDNEEKPPAPPLRMNSN (βP2; SEQ ID NO: 3) and SDNGELEDKPPAPPVRMSST (γP2; SEQ ID NO: 4).

13. A nucleic acid comprising a sequence encoding the peptide according to claim 1.

14. An expression vector comprising the nucleic acid according to claim 1.

15. A host cell transfected with the nucleic acid according to claim 13 or an expression vector comprising the nucleic acid.

16. A composition comprising the peptide according to claim 1, a nucleic acid encoding the peptide, an expression vector comprising the nucleic acid, or a host cell transfected with the nucleic acid, together with a pharmaceutically acceptable excipient or carrier.

17. A method of inhibiting an isoform of PAK kinase, the method comprising contacting an isoform of PAK kinase with the peptide according to claim 1 or with an antibody selected from the group consisting of: antibody sc-882, antibody sc-1871, antibody sc-1872 and antibody sc-7117 (Santa Cruz Biotechnology, Inc).

18. A method of stimulating neurite outgrowth or nerve regeneration, the method comprising contacting a nerve cell with an αPAK isoform specific antagonist comprising the peptide according to claim 6 or an antibody sc-882 (Santa Cruz Biotechnology, Inc).

19. A method of inhibiting axonal guidance or enabling neurite outgrowth across an attractive/repulsive boundary, the method comprising contacting a nerve cell with a γPAK isoform specific antagonist comprising the peptide according to claim 10 or an antibody sc-1872 or antibody sc-7117 (Santa Cruz Biotechnology, Inc).

20. The method according to claim 19, wherein the attractive/repulsive boundary is a laminin/CSPG boundary.

21. A method of modulating axonal guidance, the method comprising contacting a nerve cell with an isoform specific antagonist of PAK kinase comprising the peptide according to claim 1, an antibody sc-882, an antibody sc-1871, an antibody sc-1872 or an antibody sc-7117 (Santa Cruz Biotechnology, Inc).

22. A method of identifying an isoform specific antagonist of PAK kinase, the method comprising: (a) providing a candidate molecule, (b) contacting the candidate molecule with a peptide comprising an Nck binding portion of a PAK isoform, or a fragment, homologue, variant or derivative thereof, and (c) detecting binding of the candidate molecule to the peptide, fragment, homologue, variant or derivative thereof.

23. The method according to claim 22, wherein the PAK isoform is αPAK, γPAK, or both.

24. The method according to claim 22, wherein the method further comprises providing Nck, or a fragment, homologue, variant or derivative thereof, and contacting the peptide comprising an Nck binding portion of PAK, or a fragment, homologue, variant or derivative thereof with Nck, or a fragment, homologue, variant or derivative thereof in the presence of the candidate molecule, and selecting candidate molecules that modulate binding between the peptide and Nck.

25. The method according to claim 22, wherein the method further comprises isolating or synthesising a selected or identified molecule.

26. A method of identifying an isoform specific antagonist of PAK kinase, the method comprising: (a) providing a candidate molecule, (b) contacting a peptide comprising an PAK binding portion of Nck, or a fragment, homologue, variant or derivative thereof, with a candidate molecule, and (c) detecting the binding of the molecule to the PAK binding peptide, fragment, homologue, variant or derivative thereof.

27. The method according to claim 26, wherein the PAK isoform is αPAK, γPAK, or both.

28. The method according to claim 26, wherein the method further comprises providing Nck, or a fragment, homologue, variant or derivative thereof, and contacting the peptide comprising an Nck binding portion of PAK, or a fragment, homologue, variant or derivative thereof with Nck, or a fragment, homologue, variant or derivative thereof in the presence of the candidate molecule, and selecting candidate molecules which modulate binding between the peptide and Nck.

29. The method according to claim 26, wherein the method further comprises isolating or synthesising a selected or identified molecule.

30. A method of identifying an isoform specific antagonist of PAK kinase, the method comprising: (a) providing a candidate molecule, (b) contacting the candidate molecule with a peptide comprising an Nck binding portion of a PAK isoform, or a fragment, homologue, variant or derivative thereof, and (c) detecting modulation of activity of the peptide, fragment, homologue, variant or derivative thereof.

31. The method according to claim 30, wherein the PAK isoform is αPAK, γPAK, or both.

32. The method according claim 30, wherein the method further comprises providing Nck, or a fragment, homologue, variant or derivative thereof, and contacting the peptide comprising an Nck binding portion of PAK, or a fragment, homologue, variant or derivative thereof with Nck, or a fragment, homologue, variant or derivative thereof in the presence of the candidate molecule, and selecting candidate molecules which modulate binding between the peptide and Nck.

33. A method according to claim 30, wherein the method further comprises isolating or synthesising a selected or identified molecule.

34. The method according to claim 30, wherein the activity comprises a γPAK isoform specific activity selected from the group consisting of: maintenance of axonal guidance and inhibition of neurite outgrowth across an attractive/repulsive boundary.

35. The method according to claim 34, wherein the attractive/repulsive boundary is a laminin/CSPG boundary.

36. A combination of (i) a PAK isoform specific antagonist comprising the peptide according to claim 1, (ii) an antibody sc-882, an antibody sc-1871, an antibody sc-1872 or an antibody sc-7117 (Santa Cruz Biotechnology, Inc) and (iii) a nerve cell.


SEQUENCE LISTING (64 sequences)

SEQ ID NO: 1 - protein, 15 aa, Artificial Sequence
  Description of Artificial Sequence Synthetic peptide
EDKPPAPPMRNTSMI

SEQ ID NO: 2 - protein, 20 aa, Artificial Sequence
  Description of Artificial Sequence Synthetic peptide
SNNGLDIQDKPPAPPMRNTS

SEQ ID NO: 3 - protein, 20 aa, Artificial Sequence
  Description of Artificial Sequence Synthetic peptide
SDSLDNEEKPPAPPLRMNSN

SEQ ID NO: 4 - protein, 20 aa, Artificial Sequence
  Description of Artificial Sequence Synthetic peptide
SDNGELEDKPPAPPVRMSST

SEQ ID NO: 5 - protein, 9 aa, Artificial Sequence
  Description of Artificial Sequence Synthetic P1 polyproline stretch sequence
PXXPXRXXS

SEQ ID NO: 6 - protein, 543 aa, Rattus norvegicus
MSNNGLDVQDKPPAPPMRNTSTMIGAGSKDPGTLNHGSKPLPPPEEKKKKDRFYRSILAGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNTLNVKTVSETPAVPPVSEDEDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNH

SEQ ID NO: 7 - protein, 420 aa, Mus musculus
MSNNGVDIQDKPPAPPMRNTSTMIGAGSKDTGTLNHGSKPLPPNPEEKKKKDRFYRSILPGDKTNKKREKERPEISLPSDFEHTIHVGFDAVTGEFTGMPEQWARLLQTSNITKSEQKKNPQAVLDVLEFYNSKKTSNSKKYMSFTDKSAEDYNSSNTLNVKTVSETPAVPPVSEDDEDDDDDATPPPVIAPRPEHTKSVYTRSVIEPLPVTPTRDVATSPISPTENNTTPPDALTRNTEKQKKKPKMSDEEILEKLRSIVSVGDPKKKYTPFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSK

SEQ ID NO: 8 - protein, 545 aa, Homo sapiens
MSNNGLDIQDKPPAPPMRNTSTMIGVGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLDMDVEKRGSAKELLQHQFLKIAKPLSSLTPLIAAAKEATKNNH

SEQ ID NO: 9 - protein, 544 aa, Rattus norvegicus
MSDSLDNEEKPPAPPLRMNSNNRDSSALNHSSKPLPMAPEEKNKKARLRSIFPGGGDKTNKKKEKERPEISLPSDFEHTIHVGFDAVTGEFTGIPEQWARLLQTSNITKLEQKKNPQAVLDVLKFYDSKETVNNQKYMSFTSGDKSAHGYIAAHQSNTKTASEPPLAPPVSEEEDEEEEEEEDDNEPPPVIAPRPEHTKSIYTRSVVESIASPAAPNKEATPPSAENANSSTLYRNTDRQRKKSKMTDEEILEKLRSIVSVGDPKKKYTRFEKIGQGASGTVYTALDIATGQEVAIKQMNLQQQPKKELIINEILVMRENKNPNIVNYLDSYLVGDELWVVMEYLAGGSLTDVVTETCMDEGQIAAVCRECLQALDFLHSNQVIHRDIKSDNILLGMDGSVKLTDFGFCAQITPEQSKRSTMVGTPYWMAPEVVTRKAYGPKVDIWSLGIMAIEMVEGEPPYLNENPLRALYLIATNGTPELQNPERLSAVFRDFLNRCLEMDVDRRGSAKELLQHPFLKLAKPLSSLTPLILAAKEAIKNSSR

SEQ ID NO: 10 - protein, 559 aa, Mus musculus
MSDSLDNEEKPPAPPLRMNSNNRDSSALNHSSKPLPMAPEEKNKKARLRSIFPGGGDKTNKKKEKERPEISLPSDFEHTIHVGFDAVTGEFTPDLYGSQMCPGKLPEGIPEQWARLLQTSNITKLEQKKNPQAVLDVLKFYDSKETVNNQKYMSFTSGDKSAHGYIAAHQSNTKTASEPPLAPPVSEEEDEEEEEEEDDNEPPPVIAPRPEHTKSIYTRSVVESIASPAAPNKEDIPPSAENANSTTLYRNTDRQRKKSKMTDEEILEKLRSIVSVGDPKKKYTRFEKIGQGASGTVYTALDIATGQEVAIKQMNLQQQPKKELIINEILVMRENKNPNIVNYLDSYLVGDELWVVMEYLAGGSLTDVVTETCMDEGQIAAVCRECLQALDFLHSNQVIHRDIKSDNILLGMDGSVKLTDFGFCAQITPEQSKRSTMVGTPYWMAPEVVTRKAYGPKVDIWSLGIMAIEMVEGEPPYLNENPLRALYLIATNGTPELQNPERLSAVFRDFLNRCLEMDVDRRGSAKELLQHPFLKLAKPLSSLTPLIIAAKEAIKNSSR

SEQ ID NO: 11 - protein, 544 aa, Homo sapiens
MSDGLDNEEKPPAPPLRMNSNNRDSSALNHSSKPLPMAPEEKNKKARLRSIFPGGGDKTNKKKEKERPEISLPSDFEHTIHVGFDAVTGEFTGIPEQWARLLQTSNITKLEQKKNPQAVLDVLKFYDSKETVNNQKYMSFTSGDKSAHGYIAAHPSSTKTASEPPLAPPVSEEEDEEEEEEEDENEPPPVIAPRPEHTKSIYTRSVVESIASPAVPNKEVTPPSAENANSSTLYRNTDRQRKKSKMTDEEILEKLRSIVSVGDPKKKYTRFEKIGQGASGTVYTALDIATGQEVAIKQMNLQQQPKKELIINEILVMRENKNPNIVNYLDSYLVGDELWVVMEYLAGGSLTDVVTETCMDEGQIAAVCRECLQALDFLHSNQVIHRDIKSDNILLGMDGSVKLTDFGFCAQITPEQSKRSTMVGTPYWMAPEVVTRKAYGPKVDIWSLGIMAIEMVEGEPPYLNENPLRALYLIATNGTPELQNPERLSAVFRDFLNRCLEMDVDRRGSAKELLQHPFLKLAKPLSSLTPLIIAAKEAIKNSSR

SEQ ID NO: 12 - protein, 524 aa, Rattus norvegicus
MSDNGELEDKPPAPPVRMSSTIFSTGGKDPLSANHSLKPLPSVPEEKKPRNKIISIFSSTEKGSKKKEKERPEISPPSDFEHTIHVGFDAVTGEFTGMPEQWARLLQTSNITKLEQKKNPQAVLDVLKFYDSNTVKQKYLSFTPPEKDGFPSGTPALNTKGSETSAVVTEEDDDDEDAAPPVIAPRPDHTKSIYTRSVIDPIPAPVGDSNVDSGAKSSDKQKKKAKMTDEEIMEKLRTIVSIGDPKKKYTRYEKIGQGASGTVFTATDVALGQEVAIKQINLQKQPKKELIINEILVMKELKNPNIVNFLDSYLVGDELFVVMEYLAGGSLTDVVTETCMDEAQIAAVCRECLQALEFLHANQVIHRDIKSDNVLLGMEGSVKLTDFGFCAQITPEQSKRSTMVGTPYWMAPEVVTRKAYGPKVDIWSLGIMAIEMVEGEPPYLNENPLRALYLIATNGTPELQNPEKLSPIFRDFLNRCLEMDVEKRGSAKELLQHPFLKLAKPLSSLTPLILAAKEAMKSNR

SEQ ID NO: 13 - protein, 525 aa, Mus musculus [sequence omitted]

SEQ ID NO: 14 - protein, 525 aa, Homo sapiens [sequence omitted]

SEQ ID NO: 15 - protein, 60 aa, Artificial Sequence
  Description of Artificial Sequence Synthetic SH3 domain sequence
AYVKFNYMAEREDELSLIKGTKVIVMEKCSDGWWRGSYNGQVGWFPSNYVTEEGDSPLGD

SEQ ID NO: 16 - protein, 376 aa, Homo sapiens
MAEEVVVVAKFDYVAQQEQELDIKKNERLWLLDDSKSWWRVRNSMNKTGFVPSNYVERKNSARKASIVKNLKDTLGIGKVKRKPSVPDSASPADDSFVDPGERLYDLNMPAYVKFNYMAEREDELSLIKGTKVIVMEKCSDGWWRGSYNGQVGWFPSNYVTEEGDSPLGDHVGSLSEKLAAVVNNLNTGQVLHVVQALYPFSSSNDEELNFEKGDVMDVIEKPENDPEWWKCRKINGMVGLVPKNYVTVMQNNPLTSGLEPSPPQCDYIRPSLTGKFAGNPWYYGKVTRHQAEMALNERGHEGDFLIRDSESSPNDFSVSLKAQGKNKHFKVQLKETVYCIGQRKFSTMEELVEYKKAPIFTSEQGEKLYLVKHLS

SEQ ID NO: 17 - protein, 380 aa, Homo sapiens
MTEEVIVIAKWDYTAQQDQELDIKKNERLWLLDDSKTWWRVRNAANRTGYVPSNYVERKNSLKKGSLVKNLKDTLGLGKTRRKTSARDASPTPSTDAEYPANGSGADRIYDLNIPAFVKFAYVAEREDELSLVKGSRVTVMEKCSDGWWRGSYNGQIGWFPSNYVLEEVDEAAAESPSFLSLRKGASLSNGQGSRVLHVVQTLYPFSSVTEEELNFEKGETMEVIEKPENDPEWWKCKNARGQVGLVPKNYVVVLSDGPALHPAHAPQISYTGPSSSGRFAGREWYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 18 - protein, 20 aa, Artificial Sequence
  Description of Artificial Sequence Synthetic fragment peptide
SNNGLDVQDKPPAPPMRNTS

SEQ ID NO: 19 - protein, 21 aa, Artificial Sequence
  Description of Artificial Sequence Synthetic fragment peptide
SDSLDNEEKPPAPPLRMNSNN

SEQ ID NO: 20 - protein, 21 aa, Artificial Sequence
  Description of Artificial Sequence Synthetic fragment peptide
SDNGELEDKPPAPPVRMSSTI

SEQ ID NO: 21 - protein, 21 aa, Artificial Sequence
  Description of Artificial Sequence Synthetic fragment peptide
SDSLDNEEKPPAPPLRMNSNN

SEQ ID NO: 22 - protein, 20 aa, Artificial Sequence
  Description of Artificial Sequence Synthetic fragment peptide
SNNGVDIQDKPPAPPMRNTS

SEQ ID NO: 23 - protein, 21 aa, Artificial Sequence
  Description of Artificial Sequence Synthetic fragment peptide
SDNGELEDKPPAPPVRMSSTI

SEQ ID NO: 24 - protein, 21 aa, Artificial Sequence
  Description of Artificial Sequence Synthetic fragment peptide
SDGLDNEEKPPAPPLRMNSNN

SEQ ID NO: 25 - protein, 20 aa, Rattus rattus
SNNGLDVQDKPPAPPMRNTS

SEQ ID NO: 26 - protein, 20 aa, Mus musculus
SNNGVDIQDKPPAPPMRNTS

SEQ ID NO: 27 - protein, 20 aa, Homo sapiens
SNNGLDIQDKPPAPPMRNTS

SEQ ID NO: 28 - protein, 21 aa, Rattus rattus
SDSLDNEEKPPAPPLRMNSNN

SEQ ID NO: 29 - protein, 21 aa, Mus musculus
SDGLDNEEKPPAPPLRMNSNN

SEQ ID NO: 30 - protein, 21 aa, Homo sapiens
SDSLDNEEKPPAPPLRMNSNN

SEQ ID NO: 31 - protein, 21 aa, Rattus rattus
SDNGELEDKPPAPPVRMSSTI

SEQ ID NO: 32 - protein, 21 aa, Mus musculus
SDNGELEDKPPAPPVRMSSTI

SEQ ID NO: 33 - protein, 21 aa, Homo sapiens
SDNGELEDKPPAPPVRMSSTI

SEQ ID NO: 34 - protein, 9 aa, Artificial Sequence
  Description of Artificial Sequence Synthetic peptide motif
PAPPMRNTS

SEQ ID NO: 35 - protein, 9 aa, Artificial Sequence
  Description of Artificial Sequence Synthetic peptide motif
PAPPVRMSS

SEQ ID NO: 36 - protein, 9 aa, Artificial Sequence
  Description of Artificial Sequence Synthetic peptide motif
PAPPLRMNS

SEQ ID NO: 37 - protein, 9 aa, Artificial Sequence
  Description of Artificial Sequence Synthetic peptide motif
PAPPMRNTS

SEQ ID NO: 38 - protein, 9 aa, Artificial Sequence
  Description of Artificial Sequence Synthetic peptide motif
PAPPVRMSS

SEQ ID NO: 39 - protein, 9 aa, Artificial Sequence
  Description of Artificial Sequence Synthetic peptide motif
PAPPLRMNS

SEQ ID NO: 40 - protein, 9 aa, Artificial Sequence
  Description of Artificial Sequence Synthetic peptide motif
PAPPMRNTS

SEQ ID NO: 41 - protein, 9 aa, Artificial Sequence
  Description of Artificial Sequence Synthetic peptide motif
PAPPVRMSS

SEQ ID NO: 42 - protein, 9 aa, Artificial Sequence
  Description of Artificial Sequence Synthetic peptide motif
PAPPLRMNS

SEQ ID NO: 43 - protein, 12 aa, Artificial Sequence
  Description of Artificial Sequence Synthetic domain sequence
MSNNGLDVQDKP

SEQ ID NO: 44 - protein, 12 aa, Artificial Sequence
  Description of Artificial Sequence Synthetic domain sequence
MSNNGLDIQDKP

SEQ ID NO: 45 - protein, 12 aa, Artificial Sequence
  Description of Artificial Sequence Synthetic domian sequence
MSNNGVDIQDKP

SEQ ID NO: 46 - protein, 11 aa, Artificial Sequence
  Description of Artificial Sequence Synthetic domain sequence
SNNGLDVQDKP

SEQ ID NO: 47 - protein, 11 aa, Artificial Sequence
  Description of Artificial Sequence Synthetic domain sequence
SNNGLDIQDKP

SEQ ID NO: 48 - protein, 11 aa, Artificial Sequence
  Description of Artificial Sequence Synthetic domain sequence
MSDSLDNEEKP

SEQ ID NO: 49 - protein, 13 aa, Artificial Sequence
  Description of Artificial Sequence Synthetic domain sequence
MSDSLDNEEKPNN

SEQ ID NO: 50 - protein, 13 aa, Artificial Sequence
  Description of Artificial Sequence Synthetic domain sequence
MSDGLDNEEKPNN

SEQ ID NO: 51 - protein, 10 aa, Artificial Sequence
  Description of Artificial Sequence Synthetic domain sequence
SDSLDNEEKP

SEQ ID NO: 52 - protein, 12 aa, Artificial Sequence
  Description of Artificial Sequence Synthetic domain sequence
SDSLDNEEKPNN

SEQ ID NO: 53 - protein, 12 aa, Artificial Sequence
  Description of Artificial Sequence Synthetic domain sequence
SDGLDNEEKPNN

SEQ ID NO: 54 - protein, 13 aa, Artificial Sequence
  Description of Artificial Sequence Synthetic domain sequence
MSDNGELEDKPTI

SEQ ID NO: 55 - protein, 11 aa, Artificial Sequence
  Description of Artificial Sequence Synthetic domain sequence
SNNGVDIQDKP

SEQ ID NO: 56 - protein, 11 aa, Artificial Sequence
  Description of Artificial Sequence Synthetic domain sequence
MSDNGELEDKP

SEQ ID NO: 57 - protein, 12 aa, Artificial Sequence
  Description of Artificial Sequence Synthetic domain sequence
SDNGELEDKPTI

SEQ ID NO: 58 - protein, 10 aa, Artificial Sequence
  Description of Artificial Sequence Synthetic domain sequence
SDNGELEDKP

SEQ ID NO: 59 - protein, 60 aa, Artificial Sequence; identical to SEQ ID NO: 15
  Description of Artificial Sequence Synthetic SH3 domain 2 sequence

SEQ ID NO: 60 - protein, 8 aa, Artificial Sequence
  Description of Artificial Sequence Synthetic fragment peptide
FYDSNTVK

SEQ ID NO: 61 - protein, 12 aa, Artificial Sequence
  Description of Artificial Sequence Synthetic fragment peptide
GTEAPAVVTEEE

SEQ ID NO: 62 - protein, 10 aa, Artificial Sequence
  Description of Artificial Sequence Synthetic fragment peptide
SVIDPVPAPV

SEQ ID NO: 63 - protein, 15 aa, Artificial Sequence
  Description of Artificial Sequence Synthetic peptide
EDKPPAPPMRNTSMI

SEQ ID NO: 64 - protein, 6 aa, Artificial Sequence
  Description of Artificial Sequence Synthetic 6xHis tag
HHHHHH